(19) United States
(12) Patent Application Publication
(10) Pub. No.: US 2014/0243233 A1
(43) Pub. Date: Aug. 28, 2014

(54) METHODS FOR MEASURING HDL SUBPOPULATIONS

(71) Applicant: Scott W. Altmann, Asbury Park, NJ (US)

(72) Inventor: Scott W. Altmann, Asbury Park, NJ (US)

(73) Assignee: HDL Apomics LLC, Asbury Park, NJ (US)

(21) Appl. No.: 14/236,501

(22) PCT Filed: Aug. 2, 2012
(86) PCT No.: PCT/US12/49317

§ 371 Date: May 12, 2014

Related U.S. Application Data
(60) Provisional application No. 61/515,101, filed on Aug. 4, 2011.

(51) Int. Cl. G01N 33/92

(52) U.S. Cl. CPC G01N 33/92
USPC 506/9; 436/501; 435/7.92; 506/18

(57) ABSTRACT

This invention provides a capture/detection antibody-based method for measuring the amount of a high density lipoprotein (HDL) subpopulation present in a sample, wherein each particle of the HDL subpopulation being measured is characterized by the presence of a plurality of defined protein epitopes. This invention also provides related analytical and diagnostic methods, as well as kits for performing same.

This application claims priority of U.S. Provisional Application No. 61/515,101, filed Aug. 4, 2011, the contents of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to methods and kits for measuring HDL and diagnosing cardiovascular disease and other HDL-related diseases in a subject. This invention exploits the physical proximity between two protein epitopes to identify and quantify discrete HDL subpopulations present in heterogeneous mixtures, and measure changes in HDL subpopulations as a result of disease or treatment.

### BACKGROUND OF THE INVENTION

### Cardiovascular Disease and HDL

Cardiovascular disease is a leading cause of morbidity and mortality, particularly in developed nations such as the United States, Western European countries and East Asian countries. The incidence of mortality due to cardiovascular disease in these regions has decreased in last 30 years (Braunwald, E, N. Engl. J. Med. 337:1360, 1997; Hoyert, D. L., et al., “Deaths: Preliminary Data for 2003” in National Vital Statistics Reports. Hyattsville: National Center for Health Statistics, 2005; Unal B., et. al., Circulation 109:1101, 2004). Factors contributing to improved patient outcome include improved cardiovascular diagnostics, reduction of major modifiable cardiovascular risk factors and advanced medical technologies to treat acute coronary syndrome. Despite these advances, however, cardiovascular disease remains a leading cause of morbidity and mortality in developed countries (see Hoyert D. L., et al., National Vital Statistics Reports, 2005; Ueshima, H., et. al., Circulation, 118:2702, 2008).

In the end, most cardiovascular deaths result from acute coronary syndromes, including unstable angina pectoris and acute myocardial infarction (see Shah, P. K., Am. J. Cardiol., 79:17, 1997). Coronary syndromes often arise from acute coronary thrombosis, itself typically the result of disruption or rupture of the fibrous cap of a lipid-laden atherosclerotic plaque (see Munger, M. A. and Hawkins, D. W., J. Am. Pharm. Assoc., 44(Suppl 1):S5, 2003). The understanding of the mechanisms mediating atherosclerotic plaque formation, progression and subsequent rupture remains limited. At the cellular level, the pathophysiology of the disease remains in constant evolution, albeit at such a slow pace that it takes years if not decades to reveal itself in the clinical setting. From the moment the genotypic blueprint is set to the environmental inducement brought about through lifestyle choices, the disease has a beginning and an end. The factors that influence this trajectory are numerous and at the molecular level remain mostly undefined. In the absence of detailed molecular knowledge, it is safe to say that the physiological state at any juncture during the progression of this disease is different than at any other point. This can be observed experimentally as wide-ranging biological indicators such as biomarkers, cellular events and functional activities vary over the course of the disease. Particular indicators that precede disease symptoms are often referred to as risk factors that may be predictive of the pending disease state. Some predictive indicators are closely associated with the disease while others may be intrinsically involved with the disease and its development.

Involvement of plasma cholesterol in the development of atherosclerotic risk and subsequent cardiovascular disease has been validated in both human and animal models alike. Elevated LDL cholesterol and total cholesterol are directly related to an increased risk of cardiovascular disease (Anderson, K. M., et. al., JAMA 257:2176, 1987). The positive relationship between the concentration of low-density lipoprotein cholesterol (LDL-C) and the future risk of cardiovascular events has been observed in many large-scale population studies, and the benefits of reducing LDL-C levels has been proven in numerous intervention studies. The apparent effects of aggressive LDL-lowering are exemplified by various statin treatments leading to risk reductions of over 25-35%, and further declines in LDL-C levels by co-administration of drugs targeting LDL-C levels through independent mechanisms of action (including ezetimibe and resins) could result in plaque regression.

In contrast, it has been established that the risk of cardiovascular disease is inversely proportional to plasma levels of HDL-C and the major HDL apolipoprotein, apoA1 (Gordon, D. J., et al., N. Engl. J. Med 321:1311, 1989). Studies have shown that high HDL-C levels are associated with longevity (Barzilai, N., et al., JAMA 290:2030, 2003). Consistent with these findings, an abnormally low HDL-C level is a well-accepted risk factor for the development of clinically significant atherosclerosis (particularly common in men with premature atherosclerosis) (Gordon, D. J., et al., N. Engl. J. Med. 321:1311, 1989; Wilson, P. W., et al., Arteriosclerosis 8:737, 1988).

Early demonstration of the inverse relationship between HDL-C levels and cardiovascular risk can be found in the Framingham Heart Study, which showed that individuals with HDL-C levels of less than 35 mg/dL at the beginning of the study had a future coronary risk of greater than four times that of individuals with HDL-C levels over 65 mg/dL (Wilson, P. W., et al., Amer. J. Cardiol., 46:649, 1980). Other prospective population studies including PROCAM, Helsinki Heart Study and Multiple Risk Factors Intervention Trial support the view that risk associated with lower HDL-C is independent of LDL-C levels, and raising levels of HDL-C should be considered as important a therapeutic target as lowering LDL-C. The increased risk associated with a low HDL-C can be seen at all concentrations of LDL-C (Gordon, T., et. al., Am. J. Med., 62:707, 1977). Post hoc analyses of stable CHD and ACS in prospective trials indicate that both HDL-C and triglyceride levels are associated with high risk even at recommended LDL-C goals (Olsson, A. G., et. al., Eur. Heart J., 26:890 2006; Miller, M., et. al., J. Am. Coll. Cardiol., 51:724, 2008; Barter, P., et. al., N. Eng. J. Med., 357:1301, 2007). These studies suggest that for every HDL-C increase of 1 mg/dL, the risk for a CHD event is reduced by 2-5% (Chapman M. J., et al., Curr. Med. Res. Opin. 20:1253, 2004). Thus, a strategy of targeting both high LDL-C and low HDL-C is supported by the results of the INTERHEART Study which showed that the ratio of apoB to apoA1 (reflecting LDL to HDL ratio) demonstrated considerable power for predicting future myocardial infarction in a broad population of differing ethnic origin (Yusuf S., et. al., Lancet 364:973, 2004).

Despite the growing epidemiological evidence indicating that HDL-C is a cardiovascular risk marker and raising HDL-C levels can reduce that risk, ambiguity and debate continue to challenge the concept of HDL as a risk marker or therapeutic target (see Chapman, M. J., et. al., Eur. Heart J., 2011, Apr. 29 online). Large failures of HDL-modifying drug trials undermine the confidence of researchers and clinicians alike (Tall, A. R., Arterioscler. Thromb. Vasc. Biol., 27:257, 2007; Horowitz, J. D., et. al., Cardiovasc. Drug Ther., 25-69, 2011; AIM-HIGH Investigators, Am. Heart J., 161:471, 2011) and have left researchers searching for explanations.

Cholesterol numbers are expressed as different units of measurement in different countries. The United States uses milligrams as the standard for measuring cholesterol, and levels in the blood are expressed as milligrams per deciliter (mg/dL). In Canada, millimoles per liter (mmol/L) are used in measuring cholesterol numbers, and the same goes for many parts of Europe. In the United States, good cholesterol numbers for the average, healthy person are less than 200 mg/dL. Once a person gets to 200 mg/dL, he is considered to have borderline-high levels of cholesterol. At levels of over 240 mg/dL, the person is considered to have high cholesterol. In Canada and many European countries, good cholesterol numbers are those under 5.2 mmol/L. Above 5.2 mmol/L and up to 6.2 mmol/L is considered borderline high. Once a person's levels move above 6.2 mmol/L of blood, his levels of cholesterol are considered high. Sometimes, cholesterol numbers are categorized by the type of cholesterol. In the United States, LDL levels of less than 70 mg/dL are considered best for those at higher risk for developing heart disease, which corresponds to 1.8 mmol/dL in Canada and many parts of Europe. An LDL level of 100 to 129 mg/dL in the United States and 2.6 to 3.3 mmol/L is considered close to optimal for those at lower or average risk of developing heart disease. HDL-C levels are considered good at 60 mg/dL and above in the United States, and more than 1.5 mmol/L in Canada and European countries. The range from 40 to 59 mg/dL (1.3 to 1.5 mmol/L) may be considered acceptable for HDL numbers, depending on gender and other risk factors for heart disease. Anything below 50 mg/dL (1.3 mmol/L) is considered poor for women. Levels of HDL-C below 40 mg/dL (1 mmol/L) are considered poor for men.

The current version of the Framingham Risk Score was published in 2002 (see “Third Report of the National Cholesterol Education Program (NCEP) Expert Panel” Circulation, 106:3143 2002). The publishing body is the Adult Treatment Panel III (ATP III), an expert panel of the National Heart, Lung, and Blood Institute, which is part of the National Institutes of Health (NIH), USA. The Framingham/ATP III criteria were used to estimate CHD risk in the USA. Data from 11,611 patients from a very large study, the NHANES III, were used. The Risk Score is estimated using the 10-year risk for coronary heart disease (CHD). The updated version included age range, gender, total cholesterol, LDL cholesterol, HDL cholesterol, blood pressure, hypertension treatment and smoking, and it excluded diabetes, because diabetes meanwhile was considered to be a CHD Risk Equivalent. Some patients without known CHD have a risk of cardiovascular events comparable to that of patients with established CHD. Cardiology professionals refer to such patients as having a CHD Risk Equivalent. These patients should be managed as patients with known CHD. Diabetes is accepted as a CHD Risk Equivalent.

Guidelines receive regular review and constant revision compelled by ongoing and growing scientific knowledge of the disease. Recent recommendations of the European Atherosclerosis Society (EAS) Consensus Panel (see Chapman, M. J., et. al., Eur. Heart J., 32:1345, 2011) include targeting elevated low HDL-C<1 mmol/L (40 mg/dL) and/or triglyceride-rich lipoproteins (TRLs)>1.7 mmol/L (150 mg/dL). These recommendations will facilitate reduction in the substantial cardiovascular risk that persists in patients with cardiometobolic abnormalities at LDL-C goal.

The mechanisms by which HDL prevents cardiovascular disease are the subject of current scientific research. As a predictive risk factor and then as a functional contributor to atherosclerosis, the role of HDL itself likely varies during the progression of the disease and the associated physiological state of the individual. The biological functions, attributed to the lipoprotein particle population, which are important to the prevention of plaque formation, could in fact be significantly different than those HDL activities critical to reducing inflammation of the arterial wall and unrelated still to the role HDL plays during recruitment of platelets to the growing thrombus. On an individual basis, levels of these various activities likely differ. Preceding the onset of the disease, it is supposed that a state of dyslipidemia has been established which is characterized by an imbalance in favor of circulating levels of proatherogenic, cholesterol-rich apoB-containing particles rather than the antiatherogenic apoA1-containing HDL. Mechanisms related to lipoprotein disequilibrium, such as HDL-mediated protection of LDL from oxidation and lipid exchange between HDL and LDL, may be overwhelmed by such governing principals as mass action. Some believe that HDL protects against LDL oxidative modification that may be a trigger to the initiation and progression of atherosclerosis (Parthasarathy, S., et al., Biochim. Biophys. Acta, 1044:275, 1990; Barter, P. J., et al., Circ. Res. 95: 764, 2004). Others believe that the athero-protective activity of HDL comes from removing cholesterol from artery wall macrophages (Tall, A. R., et al., J. Clin. Invest., 110:899, 2002; Oram, J. F., et. al., Arterioscler. Thromb. Vasc. Biol., 23:720, 2003). Resulting endothelial dysfunction includes arterial stiffness, extracellular matrix signaling, and induced NO-dependent vasorelaxation (Havlik, R. J., et. al., Am. J. Cardiol., 87:104, 2001; Ortiz-Muñoz, G., et. al., FASEB J. 23:3129, 2009; Nofer, J. R., et. al., J. Clin. Invest. 113:569, 2004). Other studies indicate that inflammation is the key process underlying the pathology given that inflammation is a systemic response directed at decreasing toxic effects of harmful agents and repairing vessel endothelial damage (Ross, R., et. al., N. Engl. J. Med., 340:115, 1999). A variety of specific functions associated with HDL have been attributed to its anti-inflammatory activities, including prevention of endothelial inflammation, recruitment of circulating leukocytes resulting in plaque formation followed by recruitment of platelets forming a thrombus (see Toth, P. P., J. Clin. Lipidol., 4:376, 2010; Asztalos, B. F., et. al., Curr. Opin. Lipidol., 22:176, 2011).

The pleiotropic and polygenic nature of cardiovascular disease makes for complex disease etiology, which can obfuscate both prediction and diagnosis. Since the initial studies measuring HDL-C and LDL-C (Eder, H. A., Am. J. Med. 23:269, 1957), methodologies have advanced along with technology, and predictive correlations have improved with ever more complex medical statistical analysis (Modern Medical Statistics: A Practical Guide Brian S. Everitt Wiley 2003). Even so, there continues to be a necessity for improved methods for early assessment of cardiovascular disease and risk.

### The Measurement and Properties of HDL

The principal of the surrogate lipid marker cholesterol to classify and quantify lipoprotein particles has been the historical stalwart for over fifty years. Variations include calculating non-HDL-C, which accounts for cholesterol in lipoprotein classes in addition to LDL, including VLDL and intermediate density lipoproteins (IDL). An extension of this methodology uses lipoprotein cholesterol ratios such as LDL-C:HDL-C to improve clinical correlations (Grover, S. A., et. al., Epidemiology 14:315 2002) or total cholesterol:HDL-C. More recently, risk metrics have been employed such as measuring apoA1, a protein surrogate for HDL, or apoB, the surrogate marker for LDL, which may better reflect lipoprotein particle numbers rather than their cholesterol load (Knopp, R. H., Am. J. Med. 83:75 1987; Contois, J. H., et. al., Clin. Chem., 42:507, 1996; Contois, J. H., et. al., Clin. Chem., 42:515, 1996). These approaches rely on immuno-turbidimetric or -nephelometric assays (Marcovina, S. M., et. al., Clin. Chem. 39:773, 1993), provide an alternative means of measuring those lipoprotein classes, and offer a different perspective given the physiochemical nature of the lipoprotein constituent and the methods used to measure it. Lipoproteins measured using surrogate proteins rather than lipids are reported to be less susceptible to postprandial effects and fluctuations. Similarly, proponents of the apoB:apoA1 ratio believe it to be the single best predictor of coronary risk (Walldius, G., et. al., Clin. Chem. Lab Med. 42:1355, 2004; Holzmann, M. J, et al., Ann Med. 2010 Nov. 30 in press). A comprehensive prospective cohort study designed to compare the clinical utility of all said measurements and numerous ratio metric permutations was performed to investigate prediction of coronary heart disease in men and women. The study concluded that the apoB:apoA1 ratio for predicting CHD was comparable with that of traditional lipid ratios, but did not offer incremental utility over total cholesterol:HDL-C (Ingelsson, E., et. al., J. Amer. Med. Assoc., 298:776, 2007).

Other approaches to clinical measures of lipoprotein particle concentration involve sizing and counting using nuclear magnetic resonance (Otvos, J., Clin. Cardiol. 22:∥21, 1999). This method offers an additional level of resolution by expanding HDL into three particle subpopulations founded on particle diameter. This method reported discordance between individuals when comparing LDL-C and LDL particle levels which they attributed to disproportionate cholesterol distribution between large and small LDL (Otvos, J. D., et. al., J. Clin, Lipidol., 5:105, 2011). Lastly, both analytical ultracentrifugation and electrophoretic methods used in research settings have led to fractionation of HDL into several subpopulations based on distinct physiochemical property differences (Anderson, D. W., et. al., Biochim Biophys Acta 493:55, 1977, Chapman, M. J., et. al., J. Lipid Res., 22:339, 1981, Kontush, A., et. al., Arterioscler. Thromb. Vasc. Biol. 23:1881, 2003, Asztalos, B. F., et. al., Biochim. Biophys. Acta 1169:291, 1993).

Liquid chromatography-mass spectrometry (LC-MS) is also used in the study of proteomics, where again components of a complex mixture must be detected and identified in some manner. The bottom-up proteomics LC-MS approach is a common method to identify proteins and characterize amino acid sequences and post-translational modifications (Aebersold, R. and Mann, M. Nature 422:198, 2003; Chait, B. T., Science 314:65, 2006). Proteins can be purified first or the crude protein extract digested directly, followed by one or more dimensions of separating the peptides by liquid chromatography coupled to mass spectrometry (a technique known as shotgun proteomics) (Washburn, M. P., et. al., Nat. Biotechnology 19:242, 2001; Wolters, D. A., et. al., Anal. Chem. 73:5683, 2001). By comparing the masses of the proteolytic peptides or their tandem mass spectra with those predicted from a sequence database, peptides can be identified and multiple peptide identifications assembled into a protein identification (Nesvizhskii, A. I., Methods Mol. Biol. 367:87, 2007; Nesvizhskii, A. I., et. al., Nat. Methods 4:787, 2007). Samples of complex biological fluids like human serum may be run in a modern LC-MS/MS system and result in over 1000 proteins being identified, provided that the sample was first separated using physiochemical properties such as density gradient ultracentrifugation, SDS-PAGE or HPLC. Such approaches have been used to identify and quantify proteins associated with lipoprotein particle fractions HDL and LDL.

HDL has unique and measurable physiochemical properties that arise as a direct result of the quantity and relative amounts of its two major constituents, protein and lipid (Rosenson, R. S., et. al., Clin. Chem. 57:392, 2011). Both of these two common constituents can be further divided into specific molecular entities. For lipids, seven classes, including fatty acyls, glycerolipids, glycerophospholipids, sphingolipids, sterol lipids, prenol lipids, saccharolipids and polyketides, are recognized by the LIPIDS MAPS consortium (Fahy, E., et. al., J. Lipid Res., 50:S9, 2009). At the molecular level, there are ˜30,100 distinct lipid entities identified in nature of which ˜200 have been detected in fractions of HDL and are referred to as the HDL lipidome. The human plasma proteome has been curated to date to contain 1,175 distinct genes resulting in 7,614 unique protein products (Anderson, N. L., et. al., Mol. Cell. Proteomics, 3:311, 2004). The protein fraction of HDL could consist of ˜110 different members, either bound or associating with the lipoprotein particle (Karlsson, H. et. al., Proteomics 5:1431, 2005; Rezaee, F., et. al, Proteomics, 6:721, 2006: Hortin, G. L., et. al., Biochem. Biophys. Res. Commun., 340:909, 2006; Heller, M., et. al., Proteomics 5:2619, 2005; Vasair, T., et. al., J. Clin. Inv., 117:746, 2007; Davidson, W. S., et. al., Arterioscler. Thromb. Vasc. Biol. 29:870, 2009; Davidson, P., et. al., Arterioscler. Thromb. Vasc. Biol., 30:156, 2009). The specific list of proteins associated with HDL is dependent upon the methodology used to separate this lipoprotein subclass away from a serum/plasma sample prior to analysis, given that the separation methodology can result in loss or gain of constituents (Heller, M., et. al., Proteomics 5:2619, 2005; Gordon, S. M., et. al., J. Prot. Res. 9:5239, 2010). The consequence of this observation is that the proteins associated with HDL can vary as a result of the isolation technique.

The totality of all constituents in a single HDL particle combine to generate a physiochemical state. In the physiochemical state reside measurable properties including hydrodynamic radii, volume, charge, and affinity. Such properties influence migration rates used in separation technologies employed, and include, for example, density, size/charge ratio and hydrophobicity. Separation of one particle from another is a direct consequence of differences in their physiochemical states which are defined by the content of their constituents. Typical methods of separating HDL particles from other exogenous contaminants include density ultracentrifugation, gel electrophoresis, gel filtration chromatography and affinity chromatography (Mendez, A. J., et al., J. Biol. Chem. 266:10104, 1991; Guerin, M., et. al., Arterioscler. Thromb. Vasc. Biol. 21:282, 2001; Li, Z., et. al., J. Lipid Res., 35:1698, 1994; Gordon, S. M., et. al., J. Prot. Res. 9:5239, 2010; Krimbou, L., et. al. J. Lipid Res., 44:884, 2003).

HDL particle diversity and heterogeneity is a direct result of the fact that the distribution of both the lipid and protein constituents are in disequilibrium with the HDL particle population as a whole and to each other (Li, Z., et. al., J. Lipid Res., 35:1698, 1994; Kontush, A., et. al., Arterioscler. Thromb. Vasc. Biol. 24:526, 2004; deSouza J. A. et. al., Atherosclerosis 197:84, 2008; Davidson W. S, et. al. Arterioscler. Thromb. Vasc. Biol. 29:870, 2009; Garcia-Sanchez, C., et. al., Clinica Chimica Acta, 412:292, 2011). By definition, this means that any given HDL particle contains only a subset of lipidome and proteome constituents. The molar concentration of individual proteome members in the serum is much lower than that of HDL, suggesting that specific proteome members exist only in subpopulations of HDL (Anderson, L., J. Physiol. 563:23, 2005). Furthermore, it indicates that any two particles can be distinguished from each other by their lipid and protein constituents and by the relative amounts of those molecular entities. Two HDL particles containing the exact same proteome and lipidome, but differing in quantities, can be distinguished from one another by such properties as size or volume. Similarly, two particles could have similar physiochemical properties (such as size, density or migration rate) but contain very different proteome and lipidome constituents.

HDL, when considered as a single entity, is a biologically active complex that contains a plethora of functional activities. In this context, HDL is historically recognized for its antiatherogenic and vasculoprotective activities. Particular focus on its role in cholesterol efflux and reverse-cholesterol transport (RCT), as well as its anti-thrombotic, anti-inflammatory, anti-oxidative, endothelial repair and vasodilation roles, are all believed to be critical activities contributing to the beneficial and cardio-protective role this lipoprotein class plays (see Kontush, A. and Chapman M. J., Pharmacological Rev., 58:342, 2006; deGoma, E. M., et al., J. Am. Coil. Cardiol. 51:2199; 2008 Navab, M., et al. Nat. Rev. Cardiol. 8:222, 2011). A relationship between HDL and other metabolic-related diseases (including modulation of glucose metabolism, antiapoptotic activity against pancreatic beta cells, platelet function, stem cell maturation and embryogenesis) have been demonstrated. HDL also is involved in innate immunity. HDL demonstrates specific anti-infective activities (Vanhollebeke B. and Pays E., Mol. Microbiol., 76:806, 2010) and a variety of infections modulate HDL (Baker, J., et. al., J. Infect. Dis., 201:285, 2010; Barlage, S., et. al., Intensive Care Med., 35:1877, 2009). This association may be a direct consequence given the number of HDL proteome members involved in innate immunity (Vasair, T., et. al., J. Clin. Inv., 117:746, 2007) and the utilization of HDL metabolic pathways in infection mechanisms (Scarselli, E., EMBO J. 21:5017, 2002; Shi, S. T., et al., Virology 292:198, 2002).

Evidence shows that HDL particles separated from each other based on their physiochemical qualities result in an apportioning of functional activity (Kontush, A., et. al., Atheroscler. Thromb. Vascl. Biol., 24:526, 2004; Shiflett, A. M., et. al., J. Biol. Chem. 280:32578, 2005). In other words, particles of different physiochemical states preferentially contain identifiable and specific measurable functional activities. Such segregation of functional activity with physiochemical properties indicates that bioactivity is particle type-specific. Given that particle physiochemical properties are the direct consequence of the constituent lipidome and proteome associated with the particle, it may be understood that an I-IDL particle's activity is the direct result of the absolute composition of all constituents. As such, it can be inferred that measuring the particle's constituents can identify a specific biological activity of the particle once it has been defined.

One of the most important aspects of HDL particle analysis is correct collection and storage of the sample set (Dunn, W. B., et. al., Nature Protocols 6:1060, 2011). Beyond this, sample handling may result in various technical complications in a method-dependent manner. As a consequence of HDL particle population heterogeneity and the compositional nature of the particle, analytical methods used to assess HDL that depend on separation by physiochemical properties are susceptible to limitations. The separation process causes the HDL particle to degrade from its natural state in an unpredictable manner. The separation process results in the loss or gain of constituents (Whiteaker, J. R., et. al., J. Proteome Res., 6:828, 2007). The separation process does not resolve the desired end-product from contaminating materials. The separation process does not deliver the necessary precision to resolve HDL subpopulations into distinct groups of particles of identical constituents. Methods designed to limit these issues offer a refined view of HDL, the entity, and provide clearer insights into HDL biology.

### Antibodies, Antigens and Immunoassays

An antigen is any substance that the immune system can recognize as foreign. At the molecular level, an antigen is characterized by its ability bind at the antigen-binding site of an antibody. Antigens are usually proteins or polysaccharides. Polypeptides, lipids and nucleic acids can also function as antigens. Small molecules, called haptens, can also act as antigens but typically must be chemically coupled to large carrier proteins such as bovine serum albumin or keyhole limpet hemocyanin (Wu, C. and Cinader, B., J. Exp. Med. 134:693, 1971). Vaccines are examples of immunogenic antigens intentionally administered to induce acquired immunity in the recipient (Immunobiology: The Immune System in Health and Disease, 5^{th} ed., 2001; Janeway, C. A., Travers, P., Walport, M. and Shlomchik, M. J., Garland Science, NY, 2001). Although antigens are usually thought to be derived from non-self antigens, immunogens derived from host sequences can act as antigens and can induce acquired immunity which produces antibodies capable of binding host proteins.

An epitope is also known as an antigenic determinant. The part of an antibody that recognizes the antigen epitope is called the antigen-binding site of an antibody, or paratope. It is a small region in the antibody's Fv region and is approximately 15-22 amino acids, contributed from both the antibody's heavy and light chains (Immunology, 5^{th} ed., 2003 pp. 57-75; Goldsby, R., Kindt, T. J., Osborne, B. A. and Kuby, J., W. H. Freeman and Co., NY). The epitopes of protein antigens are divided into two categories, linear epitopes and conformational epitopes, based on their structure and interaction with the paratope. (Huang, J., and Honda, W., BMC Immunology 7:7, 2006). A linear epitope interacts with the paratope based on primary structure, a continuous sequence of amino acids from the antigen. In contrast, a conformational epitope is typically composed of discontinuous sections of the antigen's amino acid sequence that are brought together upon three-dimensional protein folding. These epitopes interact with the paratope based on tertiary structure and the 3-D surface shape and features of the antigen. In some instances, a conformational epitope can be composed of a continuous sequence of amino acids constrained to a specific tertiary structure. A large number of antibody-antigen interactions have conformational epitopes (Flanagan, N., Genet. Engineer. Biotech. News, 31:x 2011; Banik, S. R. and Doranz, B. J., Genet. Engineer. Biotech. News. 3:25, 2010).

Since antigens are usually proteins that are too large to bind as a whole to any antibody, only a small portion of the protein—a specific epitope—is bound by the paratope. When used to induce an adaptive immune response, one immunogenic protein results in a polyclonal B cell response producing many different antibodies to that single antigen (Immunology, 5th ed., 2003 pp. 57-75; Goldsby, R., Kindt, T. J., Osborne, B. A. and Kuby, J., W. H. Freeman and Co. NY). The protein is recognized by multiple antibodies that interact with different epitopes. These epitopes can reside in distinct regions of the protein found spatially separated from one another while in other instances, multiple, distinguishable and overlapping epitopes can be identified (Mateau, M. J., et. al., J. Gen. Virol., 71:629, 1990).

Epitope mapping is the process of identifying the binding epitope of an antibody to its target antigen (Cunningham B. C. and Wells J. A., Science 244:1081, 1989; Zhou, Y., and Chait, B. T., Anal. Chem., 66:3723, 1994; Komoda, H., et. al., J. Immunological Methods, 183:27, 1995). In some instances, the binding of one antibody to its epitope can prevent the binding of another antibody. Beyond direct overlap of two epitopes, other issues, including steric hindrance caused by neighboring antibody molecules and the distance between an antibody and the support surface, may be at fault (Bin, L., et. al., Analyst, 121:29R, 1996). Identification and characterization of the binding sites of antibodies can aid in the discovery and development of new therapeutics, vaccines, and diagnostics (Gershoni, J. M., et. al., BioDrugs, 21: 145, 2007; Epitope Mapping: a practical approach (A practical approach series), 2001; Westwood, O. M. R. and Hay, F. C., Oxford University Press, Oxford).

An analyte that binds to an antibody is often called an antigen, and assays that use an antibody to measure the analyte are referred to as immunoassays. In addition to binding specificity, the other key feature of all immunoassays is a means to produce a measurable signal in response to a specific binding. One type of assay is a homogeneous immunoassay (or less frequently called non-separation assay). These assays are designed in such a way that a binding event effects a change in the signal produced by the label. Immunoassays in which the signal is affected by binding can often be run without a separation step. Such immunoassays can frequently be carried out simply by mixing the reagents and sample and making a physical measurement. Assays of this nature may be founded in the principles of time-resolved fluorescence (TRF) and fluorescence resonance energy transfer (FRET) (Mathis, G., Clin. Chem., 39:1953, 1993; Mathis, G., J. Biomol. Screen., 4:309, 1999). The other category of immunoassay is referred to as an enzyme immunoassay (EIA) (van Weeman, B. K. and Schuurs, A. H, FEBS Lett., 15:23 1971), also known as an enzyme-linked immunosorbent assay (ELISA) (Engvall, E. and Perlman, P., Immunochemistry, 8:871, 1971). This type of assay requires that either the antigen or antibody be immobilized on any suitable rigid or semirigid support. Supports may consist of filters, chips, plates, slides, wafers, fibers, magnetic or nonmagnetic beads, gels, tubing, plates, polymers, microparticles or cylinder (Cantarero, L. A., et. al., Anal. Biochemistry, 105:375, 1980; Kellar, K. L., et. al., Cytometry, 45:27, 2001; U.S. Pat. No. 7,510,687). The substrate can have a variety of surface forms, such as wells, trenches, pins, channels, and pores to which the polypeptides are bound. For example, a chip, such as a biochip, may be a solid substrate having a generally planar surface to which a detection reagent is attached. Also, for example, a variety of chips are available for the capture and detection of lipoprotein proteome members, from commercial sources such as Ciphergen Biosystems (Fremont, Calif.), Packard BioScience Company (Meriden Conn.), Zyomyx (Hayward, Calif.), and Phylos (Lexington, Mass.). An example of a method for producing such a biochip is described in U.S. Pat. No. 6,225,047. These assays are considered separation assays, given that quantitation of binding events follows the separation of free and bound antibody-antigen complexes. Either the sample can be bound non-specifically by adsorption to the support or specifically by binding a primary (capture) antibody to the support first. Immunoassays of this variety are called indirect, sandwich and competitive ELISA. They depend on the use of an analytical reagent that is associated with the antibody and acts as a detectable label. A large variety of labels have been successfully used including, for example, radioactive elements; enzymes; fluorescent, phosphorescent, and chemiluminescent dyes; latex and magnetic particles; dye crystallites, gold, silver, and selenium colloidal particles; metal chelates; coenzymes; electroactive groups; oligonucleotides; stable radicals; and others.

Several ELISA immunoassay formats are known (Tijssen, P., Burson, R. H. and van Knippenberg, P. H. 1985, Laboratory Techniques in Biochemistry and Molecular Biology: practice and theory of enzyme immunoassays, Elsevier Scientific Publishing Co., NY). In an indirect immunoassay, the enzyme acts as an amplifier, as only a few bound enzyme-linked antibodies are needed since the linked enzyme molecule produces many signal molecules. Within common sense limitations, the enzyme can go on producing color indefinitely, but the more antigens present, the more secondary (detection) antibody with enzyme will bind, and signal will develop faster. A major disadvantage of the indirect ELISA is that immobilization of the antigen is non-specific. So, proteins in the sample may adhere to the solid support and an antigen must compete with other analytes in the sample for binding. This can result in diminished signal if the proportion of antigen in the sample is small. The direct or sandwich-ELISA provides a solution to this problem, by starting with a capture antibody which is specific for the test antigen and selectively binds a site on the antigen in a sample mixture. This approach preferably immobilizes only the desired antigen and in principle concentrates the analyte. The antigen in the unknown sample is first bound to the antibody site, and then the detection antibody binds to the capture-antibody-antigen complex. The amount of detection antibody bound to capture-antibody-antigen complex generates the measure signal. The resulting measure will be directly proportional to the concentration of the antigen. As a prerequisite for this assay format, the binding epitope for the capture antibody must be distinct from that of the detection antibody. In a competitive-ELISA, an unlabeled antibody is bound to the antigen. The antibody-antigen complex is added to an antigen coated solid-support and the unbound antibody is washed away. A labeled secondary antibody, which is capable of recognizing the primary antibody is added and generates the signal. The remaining unbound antigen in the unknown sample competes with labeled antigen to bind the antibodies. The amount of labeled antigen bound to the antibody is then measured. In this method, the response will be inversely related to the concentration of antigen in the unknown because the higher the sample antigen concentration, the weaker the signal. The primary advantage of a competitive ELISA over other formats is the ability of the assay to use crude or impure samples and still selectively bind any antigen that may be present. Some competitive ELISA formats rely on enzyme-linked antigen rather than enzyme-linked antibody. The labeled antigen competes for primary antibody binding sites with the sample antigen. The more antigens in the sample, the less labeled antigen is retained in the well and the weaker the signal. It is common that the antigen is not first positioned in the well.

Immunoassays are used to measure an analyte which is frequently contained in a complex mixture of substances. Analytes in biological liquids (for example, serum or urine) are frequently assayed using immunoassay methods (Voller, A., et. al., Bull. World Health Org., 53:55, 1976). Such assays are based on the unique ability of an antibody to bind with high specificity to one or a very limited group of molecules. Immunoassays can be carried out for either member of an antigen/antibody pair. For antigen analytes, an antibody that specifically binds to that antigen can frequently be prepared for use as an analytical reagent. When the analyte is a specific antibody, its cognate antigen can be used as the analytical reagent. In either case, the specificity of the assay depends on the degree to which the analytical reagent is able to bind to its specific binding partner to the exclusion of all other substances that might be present in the sample to be analyzed (Boscato, L. M. and Stuart, M. C., Clin. Chem., 32:1491, 1986; Boscato, L. M. and Stuart, M. C., Clin. Chem. 34:27 1988). In addition to the need for specificity, a binding partner must be selected that has a sufficiently high affinity for the analyte to permit an accurate measurement. The affinity requirements depend on the particular assay format that is used (Tijssen, P., Burson, R. H. and van Knippenberg, P. H. 1985, Laboratory Techniques in Biochemistry and Molecular Biology: Practice and Theory of Enzyme Immunoassays, Elsevier Scientific Publishing Co., NY).

Regardless of the method used, interpretation of the signal produced in an immunoassay requires reference to a calibrator that mimics the characteristics of the sample medium. For qualitative assays, the calibrators may consist of a negative sample with no analyte and a positive sample having the lowest concentration of the analyte that is considered detectable. Quantitative assays require additional calibrators with known analyte concentrations. Comparison of the assay response of a real sample to the assay responses produced by the calibrators makes it possible to interpret the signal strength in terms of the presence or concentration of analyte in the sample (Findlay, J. W. A., et. al., J. Pharmaceutical and Biomedical Analysis, 21:1249, 2000).

### SUMMARY OF THE INVENTION

This invention provides a method for measuring the amount of a high density lipoprotein (HDL) subpopulation present in a sample, wherein each particle of the HDL subpopulation being measured is characterized by the presence of a plurality of defined protein epitopes, the method comprising performing a quantitative antibody-based assay on the sample, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of the HDL subpopulation in the sample.

This invention also provides a method for measuring the amount of each of a plurality of high density lipoprotein (HDL) subpopulations present in an HDL-containing sample, wherein each particle of each of the HDL subpopulations being measured is characterized by the presence of a plurality of defined protein epitopes, the method comprising performing a quantitative antibody-based assay on the sample, wherein, for each HDL subpopulation being measured, (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of each of the HDL subpopulations present in the sample.

This invention further provides a method for determining whether a subject is afflicted with a disorder characterized by an abnormal amount of a defined high density lipoprotein (HDL) subpopulation, wherein each particle of the HDL subpopulation is characterized by the presence of a plurality of defined protein epitopes, the method comprising (a) performing a quantitative antibody-based assay on an HDL-containing sample from the subject, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of the HDL subpopulation in the subject's sample; and (b) comparing the measured amount of HDL subpopulation in the subject's sample with a known standard correlative with the presence and/or absence of the disorder, thereby determining whether the subject is afflicted with the disorder.

This invention provides a method for determining the likelihood of a subject's becoming afflicted with a disorder, wherein the disorder's likelihood of onset is characterized by an abnormal amount of a defined high density lipoprotein (HDL) subpopulation, and wherein each particle of the HDL subpopulation is characterized by the presence of a plurality of defined protein epitopes, the method comprising
(a) performing a quantitative antibody-based assay on an HDL-containing sample from the subject, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of the HDL subpopulation in the sample; and
(b) comparing the measured amount of HDL subpopulation in the subject's sample with a standard correlative with a known likelihood of the disorder's onset,
   thereby determining the likelihood of the subject's becoming afflicted with the disorder.

This invention also provides a method for measuring the success of a high density lipoprotein (HDL)-modifying treatment on a subject, wherein the treatment's success is characterized by a change in the amount of a defined HDL subpopulation, and wherein each particle of the HDL subpopulation is characterized by the presence of a plurality of defined protein epitopes, the method comprising
(a) performing a quantitative antibody-based assay on an HDL-containing sample from the subject during or after treatment, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of HDL subpopulation in the sample; and
(b) comparing the measured amount of HDL subpopulation in the subject's sample with a known standard correlative with a successful treatment outcome,
   thereby measuring the treatment's success.

This invention further provides a method for characterizing a high density lipoprotein (HDL) particle with respect to the presence of one or more sets of defined protein epitopes, the method comprising performing an antibody-based assay on a population of the HDL particles to determine the presence and/or amount of each set of the defined protein epitopes, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby characterizing the HDL particle.

This invention still further provides a method for identifying a subpopulation of high density lipoprotein (HDL) whose abnormal concentration in a subject correlates with a particular disorder, comprising
(a) measuring the amounts of one or more HDL subpopulations present in an HDL-containing sample from a subject afflicted with the disorder, wherein each particle of each of the HDL subpopulations being measured is characterized by the presence of a plurality of defined protein epitopes, the method comprising performing a quantitative antibody-based assay on the sample, wherein, for each HDL subpopulation being measured, (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amounts of the HDL subpopulations present in the subject's sample,
(b) comparing the measured amounts of HDL subpopulations in the subject's sample with a known standard correlative with the amounts of the respective HDL subpopulations present in a healthy subject, and
(c) for each of the measured HDL subpopulations, determining whether the amount of the HDL subpopulation differs from that in the known standard,
   whereby any such difference indicates that an abnormal concentration of the HDL subpopulation correlates with the disorder.

Finally, this invention provides kits for performing the instant methods described herein. Each kit comprises (i) a solid substrate suitable for use in performing an antibody-based assay; (ii) a capture antibody operably affixed to the substrate; and (iii) in a separate compartment, a detection antibody, wherein the capture and detection antibodies are directed to different protein epitopes present on each particle of a predetermined HDL subpopulation.

### BRIEF DESCRIPTION OF THE FIGURES

FIG. 1: “Solar System” rendering of an HDL particle.

This image is a hypothetical model of an HDL particle. The HDL particle is composed of two major constituents, lipids and proteins. Several major lipid classes are represented as the large shaded concentric rings and each ring reflects the percentage of a lipid which is in proportion to the relative ring area. The overall diameter of the particle can be scaled and is designed to replicate the measured diameter of an HDL particle. Proteome members are denoted by smaller circles layered on top of the lipid rings and are labeled by gene name. Each protein molecule is represented by one circle and the area of the circle is calculated to be proportional to the molecular weight of the post-translation processed mature form of the protein and does not include any mass increase resulting from glycosylation. The distance of the proteome circles from the center of the particle is intended to account for apparent affinity differences proteome members have for the lipoprotein particle. Proteome members exhibiting the lowest affinity for the particle would be arranged furthest from the center. Such proteins would be classified as having higher particle dissociation rates and are likely to exist in both an HDL particle bound and unbound state. Basic positioning of proteome members around the radius of the particle and the relative distances to each other is essentially arbitrary in this modeling view. With exception, apoA1 has been shown to exist as a dimer and is represented by two adjacent circles to reflect this observation.

Both protein and lipid constituents can vary in each particle and the density of the particle is defined by the ratio of lipid to protein. The total amounts of all constituents define the diameter, volume and charge of the particle. Variations in particle physicochemical properties are due to differences in the mix of constituents and their absolute levels. Each specific combination of constituents and their particle levels serve as a self-contained set of instructions which in turn dictates and directs the particle's physiological activities.

FIG. 2: A hypothetical representation of HDL particle subpopulation heterogeneity.

An extrapolation of an HDL particle population displayed using the solar system model and reflecting its heterogeneous nature. This figure provides a hypothetical view that is limited in scope and detail but demonstrates common particle features as well as distinct differences. The HDL particle population exhibits the disequilibrium of the proteome members and lipidome to one another and to the particle population as a whole.

Variations in lipid and protein constituents are reflected in the diameter, shading and protein patterns for each particle. Fractionation techniques can separate particles using physicochemical properties and in doing so result in the apportioning of biological activities. HDL has a large number of measured biological activities many of which associate with cardiovascular health. To reconcile all of these reported observations, a model that depends on particle heterogeneity to account for the variety of physiological activities requires that particle subpopulations, which can be defined by physicochemical characterization or permutations of constituent molecules, perform particular and specific functions. It is the totality of all particle subpopulations that contributes to cardiovascular health, and alterations in subpopulation levels or specific constituents affect particle instructional blueprints that can reflect disease phenotypes.

FIG. 3: HDL LipoPrint analysis of plasma sample.

Acrylamide gel electrophoresis of a plasma sample prepared by pooling fifty reportedly healthy individuals was used to separate lipoprotein particle fractions. HDL particles are separated principally based on particle size, with faster migration rates and larger distances from the origin for smaller sizes. Lipoprotein particles are visualized by staining with the dye, Sudan black, which quantitatively binds neutral lipids, primarily cholesteryl esters (CE). Slow migrating VLDL and LDL appear as the last peak to the far left of the chromatograph as these two classes of lipoproteins do not resolve using the HDL LipoPrint gel system. Fast migrating albumin, stained with coomassie blue, to the far right is representative of the free protein fraction in plasma. Sudan black staining of cholesterol provides a distribution profile for that lipid class across the broad HDL fraction. The HDL is subdivided into three fractions identified as large, intermediate and small which can be observed as shading differences delineated by thick black vertical lines according to the analysis software provided by the instrument's manufacturer. Peak fitting (area under the curve; AUC) is calculated by the manufacturer's software provided with the LipoPrint system to estimate relative amounts of the three subpopulations sizes.

LipoPrint gel segments are labeled 1-20 below the chromatograph. Each segment composed of a gel two millimeters in length. The entire gel is 40 millimeters in length starting from the trailing edge of VLDL/LDL peak in fraction 1 to the leading edge of free albumin peak contained primarily in fractions 18-20.

Lipoprotein particles were further isolated from each individual gel segment by buffer extraction and the isolated particles were reduced and denatured and subjected to separation by SDS-PAGE using a 4-12% gradient gel. Following transfer and immobilization on nitrocellulose, immunoblot analysis is performed to characterize the sub-fraction distribution and relative amounts of the target protein.

The middle panel depicts the immunoblot analysis using an antibody specific for apolipoprotein A-I (ab27630). Staining of apoA1 can be clearly observed in fractions 4-20 and also in fractions 2 and 3 at much lower levels following extended exposures. Each of the apoA1 containing fractions contains varying levels of apoA1 protein. The significant level of apoA1 in fractions 18-20 is indicative of apoA1 protein in very small lipoprotein particles or lipid-free protein, both of which contain undetectable levels of cholesterol. The bottom panel provides a generalized reference for categorizing particle subpopulations into assigned fractions by particle size.

This experiment demonstrates that both apoA1 protein and HDL-cholesterol exist in disequilibrium to each other. Both particle constituents are in disequilibrium to the HDL particle population as a whole. Very large particles contain large ratios of cholesterol to apoA1 and small particles contain larger ratios of apoA1 to cholesterol. Signal levels and distribution patterns for both cholesterol and apoA1 represent profile averaging effects due to pooling of the plasma sample prior to analysis. Individual samples exhibit signal heterogeneity and variations in apoA1 and cholesterol distribution across the HDL fractions.

FIG. 4: HDL Lipoprint of a plasma sample characterized by several HDL proteome members.

HDL LipoPrint electrophoresis of a pooled plasma sample divided into 10 segments. LipoPrint gel segments are labeled 1-10 below the chromatograph. Each segment is composed of a gel four millimeters in length. The entire gel is 40 millimeters in length starting from the trailing edge of the VLDL/LDL peak in fraction 1 to the leading edge of the free albumin peak contained primarily in fraction 10.

Lipoprotein particles were further isolated from each individual gel segment by buffer extraction and the isolated particles were reduced and denatured and subjected to separation by SDS-PAGE using a 4-12% gradient gel. Following transfer and immobilization on nitrocellulose, immunoblot analysis is performed to characterize the sub-fraction distribution and relative amounts of the target protein.

Various commercial antibodies (Tables 3 and 4) targeting several HDL proteome members (Table 1) were used for immunoblot analysis. The following proteome examples: apoA1 (HDL110), apoA2 (H00000336-M03), CLU (mab2937), SerpinF1 (mab1177), SerpinA1 (mab1268), KNG1 (mab15692) and SerpinF2 (mab1470) were tested and demonstrate various distribution patterns for HDL particle sub-fractions separated by particle size. The proteome distribution disequilibrium is observable with these proteome member examples which reflect both broad and restricted distribution patterns across HDL particle sub-fractions and represent profile averaging effects due to the sample consisting of pooled plasma samples from fifty individuals.

This physicochemical separation process does resolve particles into homogeneous sub-populations, and therefore fractions characterized as positive for one or more proteome member do not establish that any two proteome members reside on the same particle. Each sub-fraction still contains multiple particle species that co-migrate under these specific separation conditions, indicating that further resolution of particle sub-populations is possible.

FIG. 5: Representation of proteome distribution disequilibrium in lipoprotein particles.

Five HDL particle subpopulations are represented as circles labeled as 2b, 2a, 3a, 3b and 3c (large to small) using standard HDL particle nomenclature. Lipoprotein particles can be fractionated and identified by various physicochemical properties including size and density, but for the purpose of this example, those differences are simply illustrated by circle diameter. Attached to the perimeter of the circle is a variety of unique shapes. Five different proteins are depicted and collectively they represent the HDL proteome. Each proteome member also has two specific epitopes (shaded patches) that are considered unique to the individual protein and different from all other epitopes. The “constellation” of proteome members surrounding each of the five particles (2b, 2a, 3a, 3b, and 3c) is similar but also contains several differences. For example, one proteome member is shared by all particles (circle) while another protein (triangle) is found only on the two largest HDL particle subpopulations (2b and 2a). This drawing exhibits a set of proteome members that are in disequilibrium to the particle population and to each other.

FIG. 6: Sandwich ELISA-based measurements of lipoprotein particle proteome.

Historically, and due to the basic principles of sandwich ELISA-based measurement, the technique requires two different antibodies targeting an individual protein, which are indicated as bound to one protein (circle). The antibodies must recognize unique and non-overlapping epitopes and the binding of one antibody must not interfere with the binding of the second. One antibody, bound to a solid support, serves to capture the target protein while the second detection antibody provides the means of generating a signal. The amount of target protein bound by both antibodies should be proportional to the signal generated, thus providing a means of quantifying the protein. In this drawing, the example proteome member (circle) can exist in HDL particle-bound form or in an unbound state. In some instances, the HDL proteome member may be bound to other classes of lipoproteins such as LDL and VLDL, and displaying the proteome not bound to an HDL particle can also represent such a situation. A comparison of Tables 1 and 2 offers examples of lipoproteins for which this may be true.

Sandwich ELISA measurements such as this are incapable of discerning the bound or unbound state of the target protein unless (1) the lipoprotein particles are first separated into their prospective subpopulations prior to measurement, or (2) either the capture or detection antibody is conformation-dependent and has the capacity to bind the target protein only in instances where the protein adopts the desired conformation in a specific subpopulation-restricted manner. Using routine sandwich-ELISA methods, the quantification of the target protein is aimed at determining the total amount of the protein in the sample.

FIG. 7: Method for measuring HDL subpopulations.

This figure exemplifies several fundamental concepts demonstrating the distinct nature of the method to measure HDL subpopulations in this application. (1) This method relies on the fact that HDL proteome member distribution is in disequilibrium to each other and to the particle population as a whole. (2) The distribution of proteome members across the particle population includes individual members that are bound to all particle subpopulations and other proteome members that demonstrate varying degrees of HDL particle subpopulation restriction. (3) This sandwich ELISA methodology requires, but is not limited to, the use of one antibody to each of the proteome members to be measured.

Using the example presented and the availability of one antibody capable of recognizing each of the five HDL proteome members, a series of sandwich ELISA assays can be devised to identify different HDL subpopulations in a sample composed of a heterogeneous mixture of HDL particles. In the bottom portion of the figure, all possible proteome pairs within each of the five particle subpopulations are represented. Each particle subpopulation can be identified by the proteome pair in which both proteome members exist together on the same particle. The total number of possible pairs is a function of the number of proteome members bound. The capture antibody, which is capable of binding the target protein in the context of any particle, will produce a measurable signal only when the detection antibody is also bound to its target protein held in close proximity on the same particles where both proteome members reside.

FIG. 8: Surrogate markers for HDL subpopulations.

Set theory can be used to identify surrogate markers for specific subpopulations. Signals from paired proteome measurements in FIG. 7 are rendered using a Venn diagram to demonstrate the use of inclusion and exclusion criteria to identify specific HDL subpopulations. Five groups are labeled as 2b, 2a, 3a, 3b, and 3c. The largest lipid-rich HDL particles, commonly referred to as HDL2, consist of the two subpopulations 2b and 2a and the smaller lipid poor HDL particles, called HDL3, consist of three subpopulations 3a, 3b and 3c. Two proteome pairs can be used to identify larger HDL2 particles (intersection 2b and 2a), while the smaller more dense HDL3 particles include one proteome pair (intersection of 3a, 3b and 3c). In addition to HDL2 and HDL3 specific particles, various other proteome pairs can serve as surrogate measurements for particle subpopulations of greater homogeneity. Specific to this example are two proteome pairs restricted to the largest HDL 2b particles, and the smallest particle subpopulation contains a single proteome pair that does not exist in any other subpopulation.

This methodology permits the use of restricted proteome particle distribution to identify subpopulations of increasingly defined homogeneity, as combinations of restricted distributions can be overlapped to identify increasingly refined subsets of particles. In a similar fashion, this method offers the means to identify proteome pairs that do not typically exist in normal healthy individuals. Such is the case for one proteome pair which can be observed in the upper left hand corner of the figure. This proteome pair resides outside the boundary of all five particle subsets in the diagram. Such instances, where both proteins and applicable antibodies exist, offer the prospect of identifying surrogate markers for HDL subpopulations that are considered atypical. HDL particles and associated proteome pairs of this nature may occur as a result of underlying genetics or disease states, and this method offers a means for their identification and measurement.

This method provides a means to expand the number of particle subpopulations that can be identified by adding increasing numbers of proteome members from Table 1. Furthermore, this method can utilize the overlapping restricted distribution of two proteome members to measure expanded subsets of particles that cannot be distinguished by a single proteome member.

FIG. 9: Method provides for geometric expansion of surrogate markers for HDL subpopulations.

The use of proteome-paired signals to identify HDL subpopulations provides the prospect of geometrically expanding the repertoire of measurements for each new antibody added for use in the proteome pair sandwich ELISA. This example incorporates the drawing from FIG. 7 (upper panel) for comparison. The lower panel displays a second antibody recognizing an alternative epitope from the first on the protein designated by the circle. The substitution of a different antibody recognizing a second unique epitope on the protein results in additional sandwich ELISAs available from the same proteome pairs, resulting in an increase of the number of possible novel measurements in proportion to the number of proteome members present. Such measurements may result in no observable signal difference and in such instances can only offer independent testing of the first measurement or introducing the second antibody provides an alternative set of measurements depending on the nature of the epitope recognized. This method can increase the number of unique proteome-paired measurements by a factor equivalent to the number of proteome members bound to the particle, thus providing the means to geometrically expand the number of potential surrogate markers for an HDL particle.

FIG. 10: Method provides for expansion of surrogate markers for HDL subpopulations.

The top panel displays the components of a sandwich ELISA which include a capture antibody (Ig-C) attached to a solid support (SS). A protein antigen composed of two unique and non-overlapping epitopes and a detection antibody (Ig-D) coupled to an agent capable of producing a measurable signal (*). In some cases the role of the capture and detection antibodies can be reversed and the resulting signals from both configurations are equivalent. The success of such experimentation is often considered a validation of the assay components and the subsequent measurement they produce. A measurement of this nature is independent of other proteins in the mixture and represents a typical sandwich ELISA.

The middle panel is an illustration of a sandwich ELISA in which the roles of the antibody pair cannot be reversed and doing so will alter the absolute values of the measurement for a given sample. Excluding technical restrictions, such as the inability of the antibody to serve in the capture role due to non-productive coupling to the solid support or to act as a detection antibody as a result of loss or altered binding following labeling signal-generating agent, other molecular explanations are possible. An example is the recognition of post-translational modifications that occurs in only a percentage of the antigen being measured such as a phosphorylation event. In this instance when the Ig-C binds the common epitope to all antigen molecules and the Ig-D binds an epitope of limited distribution, a productive signal is generated only from a subset of the total antigen bound to the Ig-C. Increasing the concentration of the antigen will not alter that ratio, as the amount of non-productive antigen binding increases to the same degree as productive antigen binding until the sandwich ELISA reaches saturation. When the Ig-C and Ig-D are reversed, only the productive antigen is bound and the signal is dependent solely on the concentration of protein containing the epitope of limited distribution. The sandwich ELISA does not saturate at the same concentration of total (productive and non-productive antigen), and the difference in signal between each sandwich. ELISA goes to unity as the limited distribution epitope increased to all antigens.

The bottom panel illustrates the unique nature of this method of measuring proteome pairs, and the Ig-C and Ig-D bind epitopes on two different proteome members. In this situation, the Ig-C and Ig-D cannot be reversed for the same reasons as described for the example above but also accounts for the antigen epitope distribution within the HDL population as well as the bound/unbound considerations described in FIG. 6. This specific relational dimension cannot be captured when both the Ig-C and Ig-D interact with unique non-overlapping epitopes on the same antigen. What was a measurement of two independent antigens has been transformed into a relational intramolecular measurement which characterizes two antigens and the four antibodies involved. The eight distinct measurements of HDL subpopulations are a result of both limited epitope distribution associated with the antigen and the distribution disequilibrium of the two proteome members have to each another. Only in instances in which both epitopes exist on all proteome members in the sample and both proteome members maintain identical particle distribution profiles, including HDL particle bound and unbound fractions, does this model not hold true.

FIG. 11: A hypothetical array of Antibodies in a 96-well format to measure HDL subpopulations.

This rendering displays a collection of antibody pairs organized into ninety-six distinct measurements of HDL subpopulations. This assay construct consists of a labeled network of shaded boxes overlaid on a 96-well (circles) plate template. Plate rows are labeled with letters (A-H) to the left of the plate and columns are labeled above the plate with numbers (1-12). Each well contains two boxes located in diagonal corners. The upper left box identifies a capture antibody by proteome and epitope using a letter and number code. The box in the lower right corner identifies the detection antibody by proteome and epitope using the letter and number code.

Labeling of proteome epitopes is essentially arbitrary, but in this example, the boxes labeled with the letter “Z” represent a non HDL proteome cardiovascular control. Proteome members are designated by a letter (A-J) and unique epitopes by a number. In this illustration, eight antibodies targeting proteome member A contribute to fifty-one sandwich ELISA measurements. Eighteen of these pairs are designed to measure proteome member A to itself using unique and non-overlapping epitopes. Thirty-three measurements utilizing antibodies to proteome member A also involve antibodies to other proteome members. In real terms proteome member A would likely be apoA1 and antibodies selected for their ability to recognize both conformational-dependent and -independent epitopes. Antibodies could serve strictly as Ig-C (A1, A3) or as Ig-D (A2), while others could serve in both roles (A4-A8). Similar design rules would hold true for other HDL proteome members. Some assays (A1/A4, A1/A5, A1/A6, and A1/A7) utilize a single Ig-C and four different Ig-D. Other assays (A4/A2, A5/A2, A6/A2 and A7/A2) utilize different Ig-C and a common Ig-D while other assays (A6/A7, A7/A6) use the same antibody pair with roles reversed. A similar design would be used to measure to proteome members to each other (A9/B2, A9/B3, A4/B2, A4/B3, A5/B1, A5/B3, A6/B4, A7/B4, B1/A9, B3/A9, B1/A2, B3/A2, B2/A5, B3/A5, B4/A6 and B4/A7) where proteome member B would represent apoA2. The remaining wells on the plate depict series of antibody combinations that target various proteome members (Table 1), all of which would have far more limited particle distribution profiles than apoA1 or apoA2.

This method is designed to construct a measurement-matrix for determining the amount of HDL subpopulations in an HDL-containing sample by performing a quantitative assay on a plurality of HDL proteome epitopes. This systematic analysis utilizes the distribution disequilibrium found between two proteome members within the HDL subpopulations and exploits the relationship those epitopes have through the use of common antibodies in multiple sandwich ELISA assays. This method replaces the typical independent intermolecular measurements of sandwich ELISA, where both antibodies interact with unique epitopes on a single protein, with a series of relational intramolecular measurements based on many antibodies used in various combinations in multiple assays.

FIG. 12: Determining whether a subject is afflicted with a disorder characterized by an abnormal amount of an HDL subpopulation.

Subject samples containing HDL are measured using the antibody array matrix to quantify the relative levels of HDL subpopulations defined by the combination of antibody pairs. Ninety-six measurements generate signal intensity levels which are reflected by grayscale shade from least (white) to greatest (black). The subpopulation profile for the subject is the composite view of multiple HDL proteome ELISA signals taken concurrently. The levels and patterns are hypothetical and offer a visual representation of measured differences in samples from individuals afflicted with diseases that affect HDL proteome member levels or their association with HDL particle subpopulations.

Samples from individuals afflicted with disorders characterized by abnormal amounts of an HDL subpopulation or abnormal levels of a proteome member can generate signal levels that are higher or lower than that of a healthy individual. Two diseases, atherosclerosis and type-2 diabetes, are examples of afflictions affecting HDL proteome levels (Kontush, A., et. al., Arterioscler. Thromb. Vasc. Biol. 24:526, 2004; Lyons, T. J., et. al. Invest. Opthomology and Visual Sci. 45:910, 2004; Vaisar, T., et. al., J. Clin. Inv., 117:746, 2008; Green, P. S., et al., Circulation 118:1259, 2008). It is unclear from existing data whether the observed changes in measured protein reflect modulations of protein on the HDL particles or changes in levels of HDL particles containing those proteins (Corsetti, J. P., et. al., PLOS One 7:e39110, 2012). This subtle difference represents a key feature in assessing HDL and an important dimension that this method brings to correlating HDL to disease states.

FIG. 13: Detection of HDL using a Sandwich ELISA assay.

Twenty-six antibodies directed at twelve HDL proteome members from Table and one non-immune antibody control (IgG-C) are labeled along the ordinate and abscissa. Cells (shaded) representing a tested sandwich ELISA can be identified by pairing a capture (ordinate) and a detection (abscissa) antibody using HDL-containing samples. Antibodies designated by their catalogue number according to Tables 3 and 4 as well as their clone identification ([clone]) where applicable, are grouped according to the proteome member they target. Sandwich ELISA assays generating a strong signal (dark), weak (intermediate) and no signal (“N” light). Cells (white) indicate antibody pairs not tested. All antibodies were evaluated by immunoblot analysis with HDL samples to confirm their capacity to recognize their cognate proteome member prior to sandwich ELISA testing.

Excluding the IgG-C non immune control, these antibodies comprise a possible 650 unique antibody pairs if a single antibody cannot serve in both the capture and detection role. Of these, 92 sandwich ELISA's were performed, of which 56 generated measurable signals and 36 did not. Some antibodies performed either capture or detection roles. Other antibodies did not work in either position despite pairing with antibodies validated to work in this assay format. Several sandwich ELISA assays utilizing antibody pairs interacting with epitopes on the same protein (apoA1, apoB, apoE) generated signals. Several detection antibodies demonstrated the capacity to work with multiple capture antibodies targeting the same proteome member, and multiple capture antibodies worked with a common detection antibody. Antibody pairs targeting different proteome members exhibited signals indicating proximity of both proteins on the same particle. Limited testing of antibodies derived from Tables 3 and 4 identified sandwich ELISA assays that place the following proteome members on the same particle: apoA1/apoA2, apoA1/apoB, apoA1/apoE, apoA1/CLU, apoA1/KNG1, apoA1/SerpinA1, apoA1/SerpinC1, apoA1/SerpinF1, and constitute novel ELISA-based measurements of HDL not previously observed.

Antibodies are commercially available and not previously evaluated for use in this sandwich ELISA format. It is expected that not all antibodies or antibody combinations should work. In some instances, technical limitations such as non-productive coupling to the solid support or labeling with a signal generating molecule may be an issue. In other cases, sandwich ELISA-validated antibodies were unable to pair and may reflect epitope availability problems or that both epitopes do not exist on the same particle. Instances of steric hindrance due to overlapping epitopes are also likely.

### DETAILED DESCRIPTION OF THE INVENTION

This invention provides an accurate tool for measuring HDL in a sample. The invention is useful for determining whether a subject is at risk of developing, is suffering from or is shifting between cardiovascular disorders. The methods are based on the physical relationship between two distinct proteins or epitopes held in proximity to one another as part of a single lipoprotein particle. That is, this invention exploits the physical proximity between two protein epitopes to identify and quantify discrete HDL subpopulations present in heterogeneous mixtures, and measure changes in HDL subpopulations as a result of disease or treatment.

### DEFINITIONS

In this application, certain terms are used which shall have the meanings set forth as follows.

As used herein, the term “antibody” includes, without limitation, (a) an immunoglobulin molecule comprising two heavy chains and two light chains and which recognizes an antigen; (b) polyclonal and monoclonal immunoglobulin molecules; and (c) monovalent and divalent fragments thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof. Antibodies may be human, humanized or nonhuman.

As used herein, the term “capture antibody” includes, for example, the primary antibody used in a homogeneous immunoassay or an ELISA immunoassay. The capture antibody is immobilized on a solid support, such as a polystyrene microtiter plate, bead or cylinder.

As used herein, the term “cardiovascular disease”, also referred to as “cardiovascular disorder” and “CVD”, includes, without limitation, heart and blood vessel diseases, such as atherosclerosis, coronary heart disease, cerebrovascular disease, and peripheral vascular disease. Cardiovascular disorders also include, for example, myocardial infarction, stroke, angina pectoris, transient ischemic attacks, and congestive heart failure. Cardiovascular disease, such as atherosclerosis, usually results from the accumulation of fatty material, inflammatory cells, extracellular matrices and plaque. Clinical symptoms and signs indicating the presence of CVD may include one or more of the following: chest pain and other forms of angina, shortness of breath, sweatiness, Q waves or inverted T waves on an EKG, a high calcium score by CT scan, at least one stenotic lesion on coronary angiography, and heart attack.

As used herein, the term “defined protein epitope” includes, without limitation, an epitope defined structurally (e.g., by primary amino acid sequence and/or atomic coordinates) and/or functionally (e.g., able to bind to a defined monoclonal antibody, ideally with a K_{d} of 10⁻⁸M or lower).

As used herein, the term “detection antibody” includes, for example, the secondary antibody used in a homogeneous immunoassay or an ELISA immunoassay. The detection antibody is typically immobile, and contains a label that produces a measurable signal.

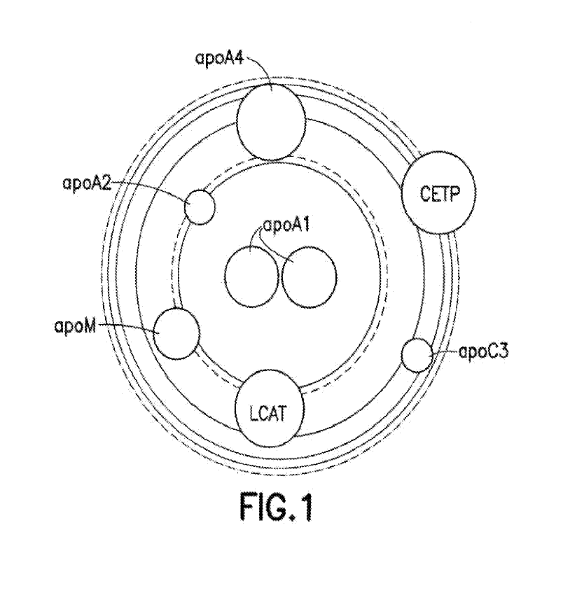

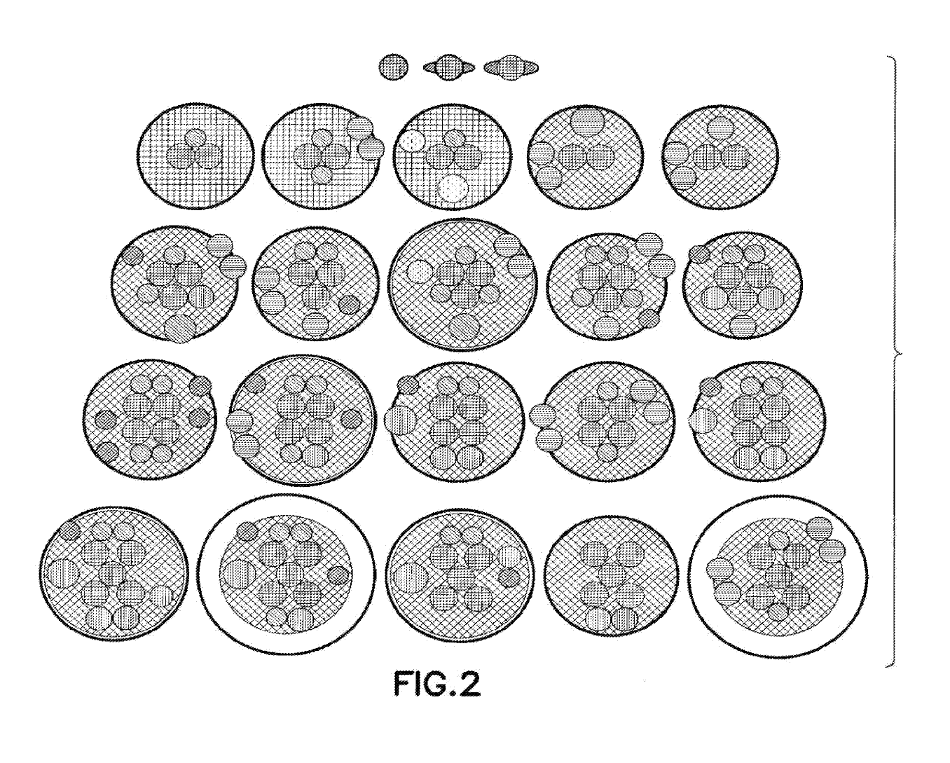

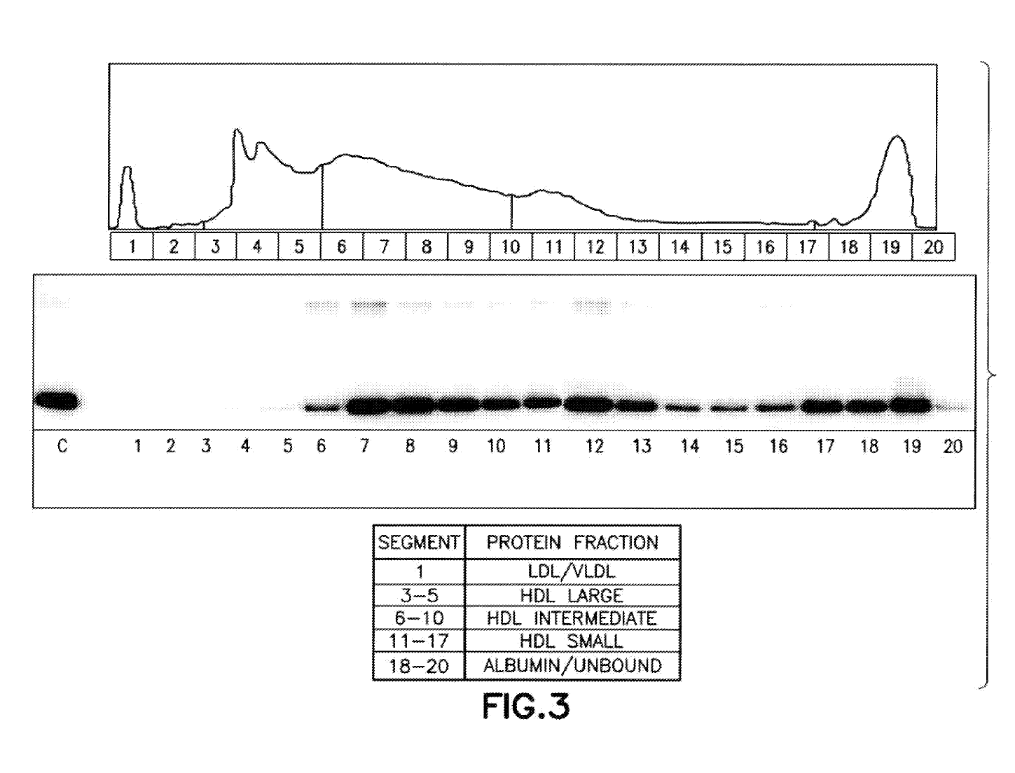

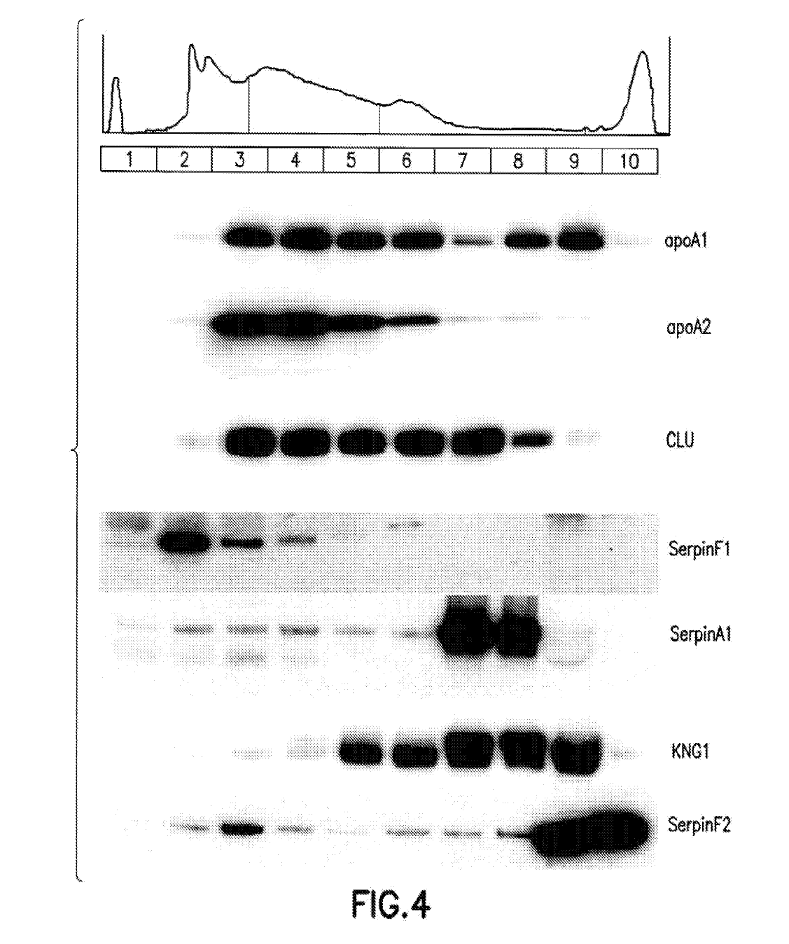

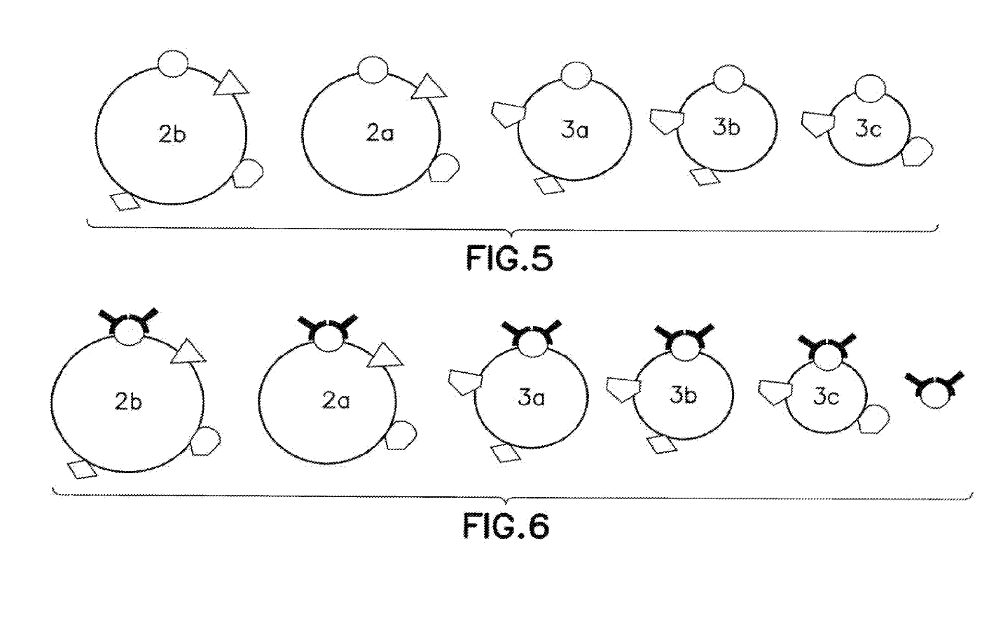

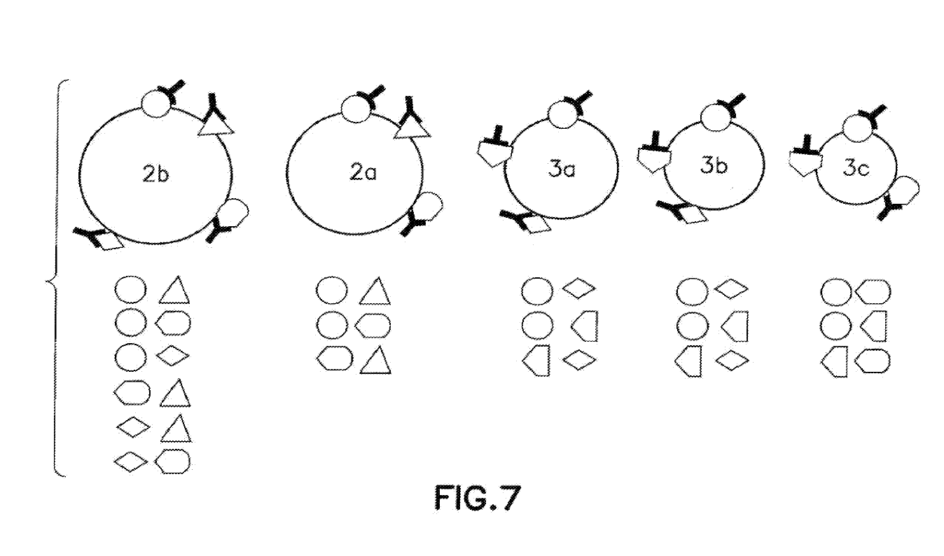

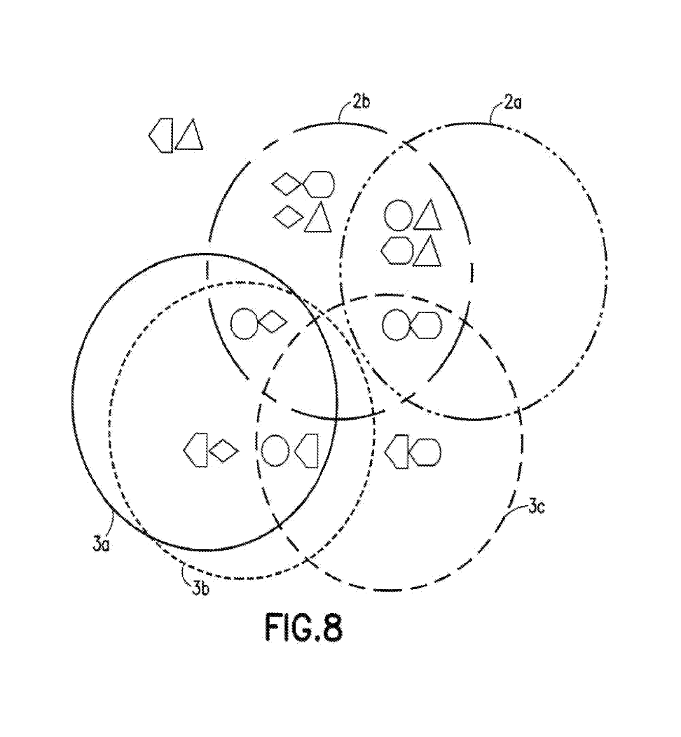

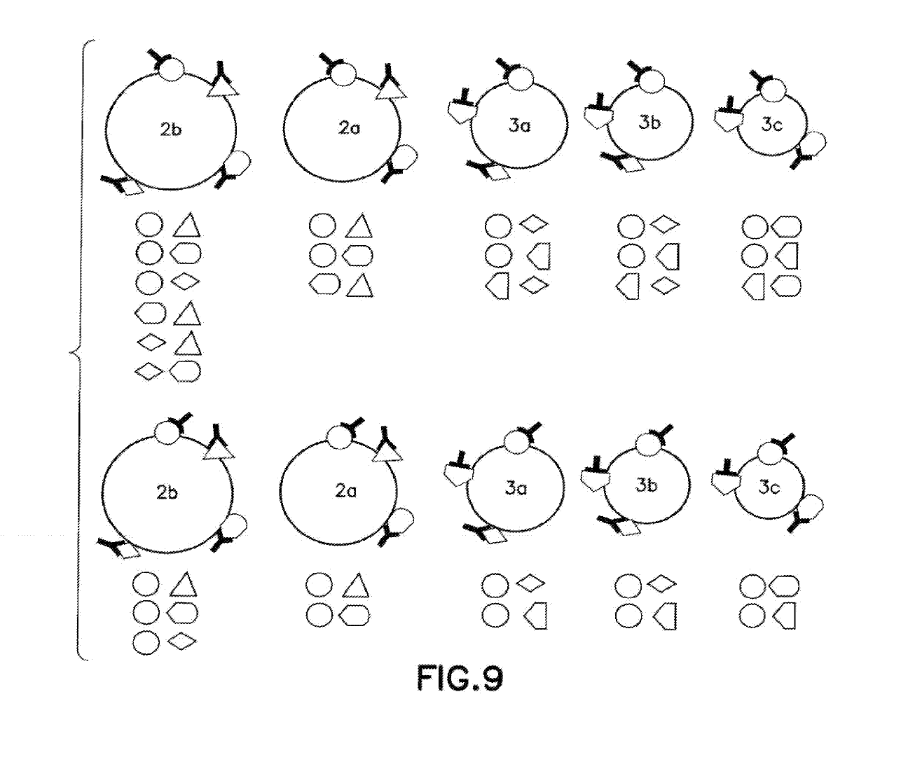

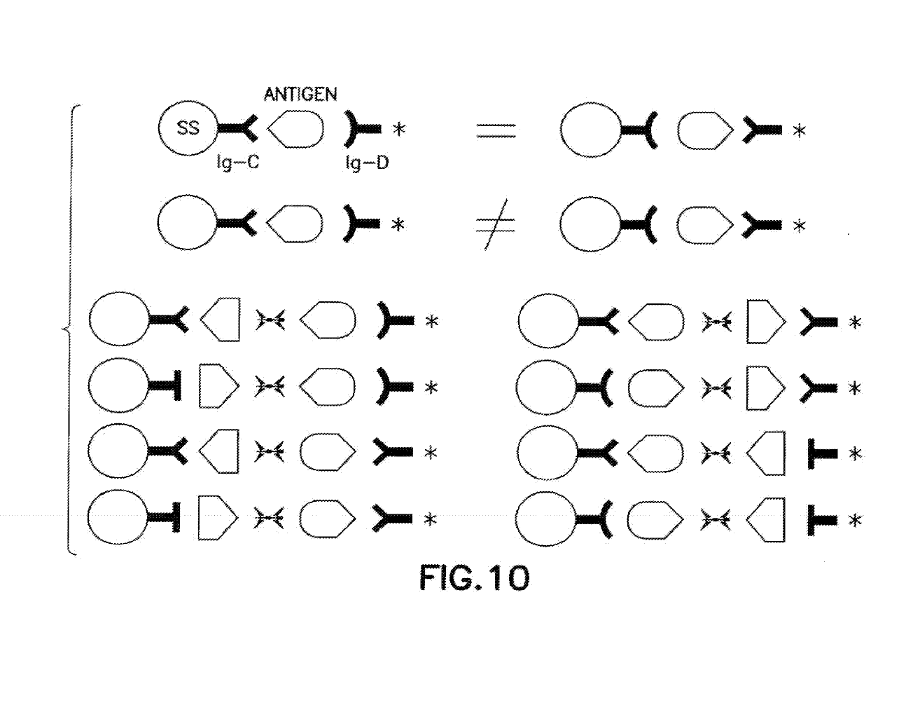

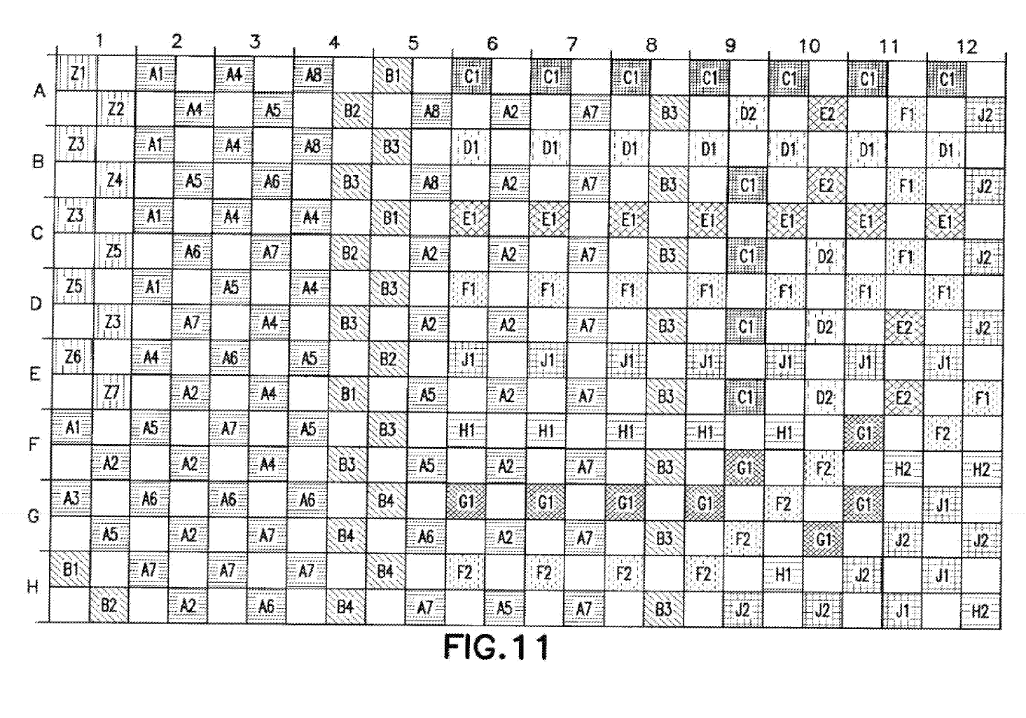

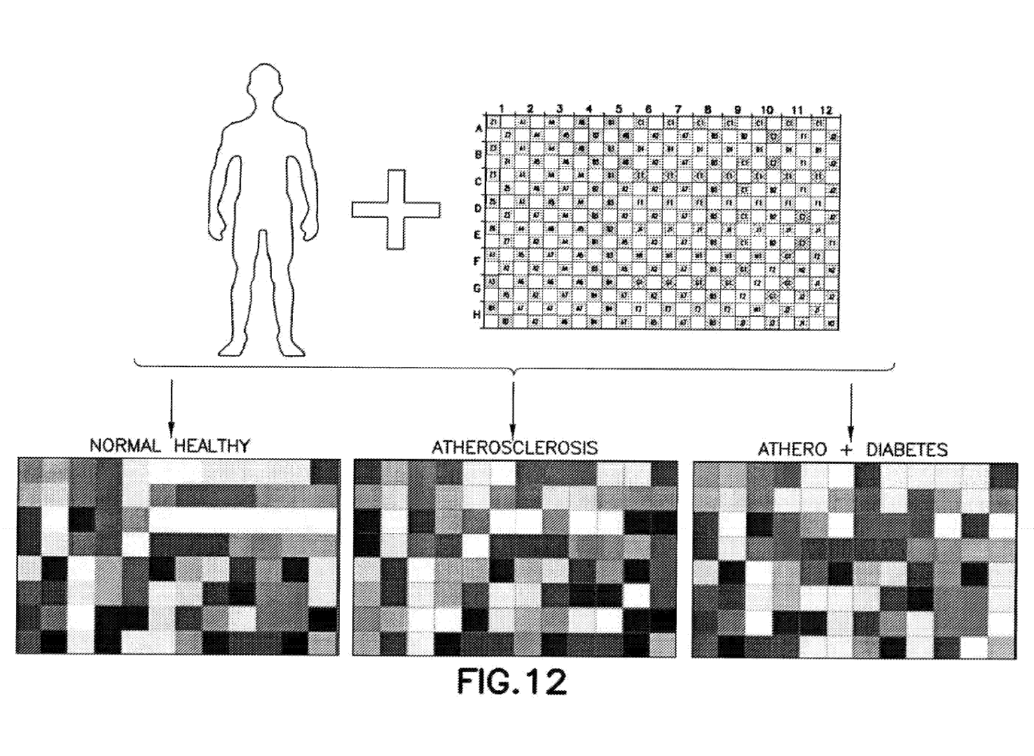

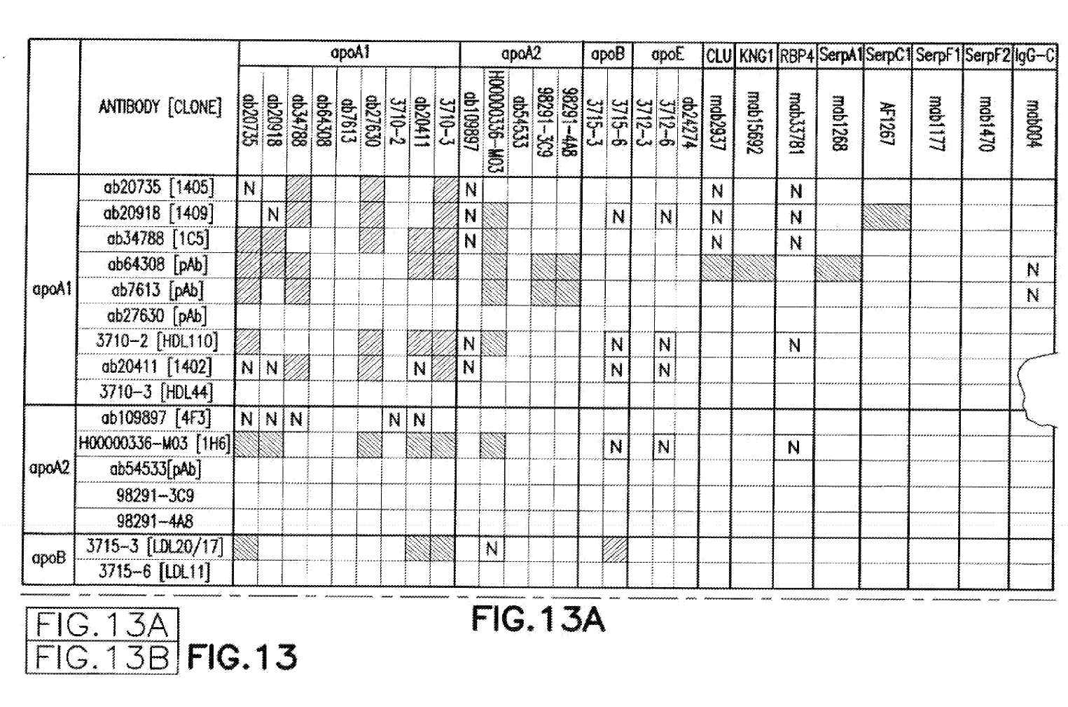

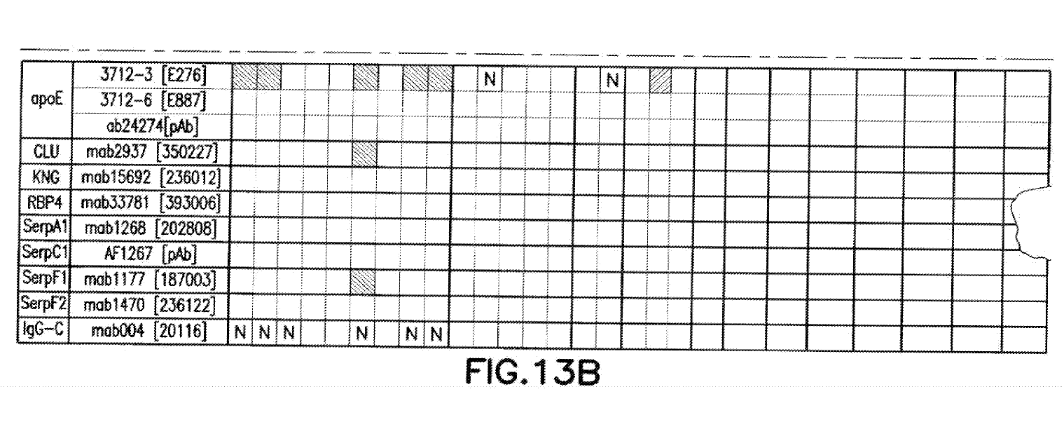

As used herein, the term “high density lipoprotein”, also referred to as “HDL”, includes, without limitation, a particle as exemplified in FIG. 1 that is made from protein and lipid, and that (i) has a density of from 1.06 to 1.21 g/mL, (ii) has a diameter from 7.1 nm to 12.6 nm, and (iii) contains at least one of apoA1, apoA2 and apoE (alternatively referred to as ApoA1, ApoA2 and ApoE, respectively). Examples of HDL include HDL3 (having a density of from 1.06 to 1.10 g/mL), and HDL2 (having a density from 1.10 to 1.21 g/mL).

As used herein, the term “HDL subpopulation” means a subset of all HDL. Preferably, the HDL subset differs from all other HDL subsets by the presence or absence of a particular protein or protein epitope.

As used herein, the term “sample”, when used with respect to HDL, includes any biological substance present within, or obtainable from, a subject. These substances include, without limitation, blood, bone marrow, urine, saliva, synovial fluid, cerebrospinal fluid or tissue, lesions, ulcers and tumors. Samples may optionally be treated, purified and/or fractionated. For example, when a sample is obtained via fractionation, the fractionation of components may take place in column chromatography by a difference in affinity between a stationary phase and a mobile phase, or by the principals of a gradient. Other fractionation methods include separation by differences in mass, solubility or density that may be induced by methods such as freezing, pH change, organic extraction, precipitation or electrophoretic mobility.

As used herein, the term “subject” includes, without limitation, a mammal such as a human, a non-human primate, a dog, a cat, a horse, a sheep, a goat, a cow, a rabbit, a pig and a rodent.

### EMBODIMENTS OF THE INVENTION

This invention provides a method for measuring the amount of a high density lipoprotein (HDL) subpopulation present in a sample, wherein each particle of the HDL subpopulation being measured is characterized by the presence of a plurality of defined protein epitopes, the method comprising performing a quantitative antibody-based assay on the sample, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of the HDL subpopulation in the sample.

The HDL subpopulation measured by this method can be characterized by any naturally occurring permutation of proteins within the HDL proteome. Members of the HDL proteome are set forth, for example, in Table 1. In a preferred embodiment, the HDL subpopulation being measured is characterized by the presence of ApoA1 protein, ApoA2 protein and/or ApoE protein. Typically, amounts of HDL in a sample such as blood are measured in mg/dL. Moreover, the “amount” of HDL subpopulation measured by this method can be either the absolute amount (e.g., 10 mg of HDL per dL of blood) or a relative amount (e.g., 1.5 times the concentration of HDL present in normal blood).

Examples of samples containing the HDL subpopulation being measured are set forth above. In a preferred embodiment, the sample is blood, plasma, serum or urine, all preferably from a human.

In this method, the plurality of defined protein epitopes can be present on the same protein. In this scenario, the plurality of defined protein epitopes are preferably present on one of ApoA1 protein, ApoA2 protein and ApoE protein. Alternatively, the plurality of defined protein epitopes can be present on two or more proteins. In this scenario, the plurality of defined protein epitopes are preferably present on two or more proteins in the HDL proteome set forth in Table 1.

The subject invention employs antibody-based assays to measure HDL subpopulations. Such methods and the antibodies they employ are well known in the art, and are exemplified above. Moreover, the antibodies that can be used in this invention are also well known, and are exemplified in Tables 3 (anti-apoA1 antibodies) and 4 (antibodies directed to various members of the HDL proteome). In one embodiment, the quantitative antibody-based assay is a radioimmunoassay (RIA) or an enzyme immunoassay (EIA). Preferably, the EIA is an enzyme-linked immunosorbent assay (ELISA), a homogeneous time resolved fluorescence assay (HTRF) or an electrochemiluminescence assay (ECL).

This invention also provides a method for measuring the amount of each of a plurality of high density lipoprotein (HDL) subpopulations present in an HDL-containing sample, wherein each particle of each of the HDL subpopulations being measured is characterized by the presence of a plurality of defined protein epitopes, the method comprising performing a quantitative antibody-based assay on the sample, wherein, for each HDL subpopulation being measured, (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of each of the HDL subpopulations present in the sample.

Envisioned is a method wherein a large number of HDL subpopulations is measured concurrently (as are biomolecules using known chip array technology) or in close temporal succession. The number of HDL subpopulations measured by this method can be any number, such as 100, 500, 1,000, 10,000, or more. In one embodiment, the number of HDL subpopulations measured is at least 16. Preferably, the number of HDL subpopulations measured is at least 96.

In this method, the amounts of HDL subpopulations can be measured either sequentially or concurrently. However, the method preferably involves concurrently measuring the amount of each of the plurality of HDL subpopulations present in the HDL-containing sample.

Also, in this method, the HDL subpopulations being measured can constitute any collection of subpopulations (e.g., grouped by disease state or characterizing proteins). In a preferred embodiment, at least one of the HDL subpopulations being measured is characterized by the presence of ApoA1 protein, ApoA2 protein and/or ApoE protein.

This invention further provides a method for determining whether a subject is afflicted with a disorder characterized by an abnormal amount of a defined high density lipoprotein (HDL) subpopulation, wherein each particle of the HDL subpopulation is characterized by the presence of a plurality of defined protein epitopes, the method comprising (a) performing a quantitative antibody-based assay on an HDL-containing sample from the subject, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of the HDL subpopulation in the subject's sample; and (b) comparing the measured amount of HDL subpopulation in the subject's sample with a known standard correlative with the presence and/or absence of the disorder (e.g., HDL measurements previously taken from healthy and afflicted subjects), thereby determining whether the subject is afflicted with the disorder.

In this method, the amount of defined HDL in an afflicted subject can be either higher or lower than in a healthy subject. In one embodiment, the amount of the defined HDL subpopulation in an afflicted subject is higher than (e.g., by 5%, 10%, 20%, 50%, 100%, or more) the amount of the defined HDL subpopulation in a healthy subject. In this scenario, the disorder can be, for example, dyslipidemia, hypertension, diabetes mellitus, coronary artery disease (CAD) or coronary heart disease (CHD).

In another embodiment, the amount of the defined HDL subpopulation in an afflicted subject is lower than (e.g., by 5%, 10%, 20%, 50%, or more) the amount of the defined HDL subpopulation in a healthy subject. In this scenario, the disorder can be, for example, dyslipidemia, atherosclerosis, diabetes mellitus, obesity-induced dyslipidemia, coronary artery disease (CAD), coronary heart disease (CHD) or chronic kidney disease (CKD).

This invention still further provides a method for determining the likelihood of a subject's becoming afflicted with a disorder, wherein the disorder's likelihood of onset is characterized by an abnormal amount of a defined high density lipoprotein (HDL) subpopulation, and wherein each particle of the HDL subpopulation is characterized by the presence of a plurality of defined protein epitopes, the method comprising
(a) performing a quantitative antibody-based assay on an HDL-containing sample from the subject, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of the HDL subpopulation in the sample; and
(b) comparing the measured amount of HDL subpopulation in the subject's sample with a standard correlative with a known likelihood of the disorder's onset (e.g., HDL measurements previously taken from healthy, at-risk and/or afflicted subjects),
   thereby determining the likelihood of the subject's becoming afflicted with the disorder.

In one embodiment, the amount of the defined HDL subpopulation in a subject likely to become afflicted is higher than (e.g., by 5%, 10%, 20%, 50%, 100%, or more) the amount of the defined HDL subpopulation in a subject less likely to become afflicted. In this scenario, the disorder can be, for example, dyslipidemia, hypertension, diabetes mellitus, coronary artery disease (CAD) or coronary heart disease (CHD).

In another embodiment, the amount of the defined HDL subpopulation in a subject likely to become afflicted is lower than (e.g., by 5%, 10%, 20%, 50%, or more) the amount of the defined HDL subpopulation in a subject less likely to become afflicted. In this scenario, the disorder can be, for example, dyslipidemia, atherosclerosis, diabetes mellitus, obesity-induced dyslipidemia, coronary artery disease (CAD), coronary heart disease (CHD) or chronic kidney disease (CKD).

This invention also provides a method for measuring the success of a high density lipoprotein (HDL)-modifying treatment on a subject, wherein the treatment's success is characterized by a change in the amount of a defined HDL subpopulation, and wherein each particle of the HDL subpopulation is characterized by the presence of a plurality of defined protein epitopes, the method comprising
(a) performing a quantitative antibody-based assay on an HDL-containing sample from the subject during or after treatment, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of HDL subpopulation in the sample; and
(b) comparing the measured amount of HDL subpopulation in the subject's sample with a known standard correlative with a successful treatment outcome,
   thereby measuring the treatment's success.

The treatment whose success is measured by this method can be any form of treatment, whether pharmaceutical or otherwise (e.g., lifestyle changes and surgery). Pharmaceutical treatments include, for example, cholesterol-lowering medications, antiplatelet agents (e.g., aspirin, ticlopidine, clopidogrel), glycoprotein IIb-IIIa inhibitors (such as abciximab, eptifibatide or tirofiban), antithrombin drugs (blood-thinners such as heparin), beta-blockers, nitrates (e.g., nitroglycerin), calcium-channel blockers, and medications for reducing blood pressure (e.g., ACE inhibitors and diuretics).

In a preferred embodiment, the HDL-modifying treatment is the administration of a statin. Statins are well known in the art, and include, for example, atorvastatin (Lipitor® and Torvast®), fluvastatin (Lescol®), lovastatin (Mevacor®, Altocor®, Altoprev®), pitavastatin (Livalo®, Pitava®), pravastatin (Pravachol®, Selektine®, Lipostan, rosuvastatin (Creston®) and simvastatin (Zocor®, Lipex®), ezetimibe/simvastatin (Vytorin®, Ezetrol®).

By way of example, in a post hoc cohort study, statins were shown to raise HDL (measured as HDL-cholesterol (HDL-C) and apoA1), and these elevations were maintained in the long-term (McTaggart, F. and Jones, P., Cardiovasc. Drugs Ther. 22:321, 2008). In patients afflicted with hypercholesterolemia, statins raise HDL-C by approximately 4% to 10%, with the percentage change greatest in patients having low HDL-C baseline levels (including patients having the common combination of high triglycerides (TG) and low HDL-C). Another study compared the effects of five different statins (namely, atorvastatin, simvastatin, pravastatin, lovastatin and fluvastatin) on the lipid, lipoprotein, and apoA1-containing high-density lipoprotein (HDL) subpopulation profiles of 86 coronary heart disease (CHD) patients (Asztalos, B. F., et al., Atherosclerosis 164:361, 2002). This study identified the most effective agents for altering the HDL subpopulation profiles in CHD patients to more closely resemble those found in healthy individuals. Finally, in patients afflicted with coronary artery disease, 12 months of combined atorvastatin and extended-release niacin therapy partially reversed the adverse changes in HDL3 protein composition (Green, P. S., et. al, Circulation 118:1259, 2008).

This invention further provides a method for characterizing a high density lipoprotein (HDL) particle with respect to the presence of one or more sets of defined protein epitopes, the method comprising performing an antibody-based assay on a population of the HDL particles to determine the presence and/or amount of each set of the defined protein epitopes, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby characterizing the HDL particle.

This method can be used to characterize any type of HDL particle. In a preferred embodiment, the antibody-based assay is performed on a population of the HDL particles selected from HDL2a, HDL2b, HDL3a, HDL3b, HDL3c, pre-β1 and pre-β2.

This invention still further provides a method for identifying a subpopulation of high density lipoprotein (HDL) whose abnormal concentration in a subject correlates with a particular disorder, comprising
(a) measuring the amounts of one or more HDL subpopulations present in an HDL-containing sample from a subject afflicted with the disorder, wherein each particle of each of the HDL subpopulations being measured is characterized by the presence of a plurality of defined protein epitopes, the method comprising performing a quantitative antibody-based assay on the sample, wherein, for each HDL subpopulation being measured, (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amounts of the HDL subpopulations present in the subject's sample,
(b) comparing the measured amounts of HDL subpopulations in the subject's sample with a known standard correlative with the amounts of the respective HDL subpopulations present in a healthy subject, and
(c) for each of the measured HDL subpopulations, determining whether the amount of the HDL subpopulation differs from that in the known standard,
   whereby any such difference indicates that an abnormal concentration of the HDL subpopulation correlates with the disorder.

This method is, in essence, a way to find novel correlations between particular disorders and HDL subpopulations. Each correlation can then form the basis for a diagnostic test for such disorder, whereby an abnormal concentration of the relevant HDL subpopulation indicates an affliction with the disorder. In a preferred embodiment, the disorder is dyslipidemia, obesity-induced dyslipidemia, hypertension, diabetes mellitus, coronary artery disease (CAD), coronary heart disease (CHD), vascular inflammation, atherosclerosis or chronic kidney disease (CKD).

Finally, this invention provides kits for performing the instant methods described herein. Each kit comprises (i) a solid substrate suitable for use in performing an antibody-based assay; (ii) a capture antibody operably affixed to the substrate; and (iii) in a separate compartment, a detection antibody, wherein the capture and detection antibodies are directed to different protein epitopes present on each particle of a predetermined HDL subpopulation.

Antibody-based diagnostic kits of all types and their methods of manufacture and use are well known. In a preferred embodiment, the instant kit is suitable for performing a radioimmunoassay (RIA) or an enzyme immunoassay (EIA). Preferably, the EIA is an enzyme-linked immunosorbent assay (ELISA), a homogeneous time resolved fluorescence assay (HTRF) or an electrochemiluminescence assay (ECL). The inclusion of suitable solvents and instructions for using these kits is envisioned.

In a preferred embodiment of the subject kits, the capture antibody is directed to an epitope present on a protein set forth in Table 1, and the detection antibody is directed to an epitope present on one of ApoA1 protein, ApoA2 protein and ApoE protein, wherein the capture and detection antibodies are directed to different epitopes. In another preferred embodiment, the capture antibody is directed to an epitope present on one of ApoA1 protein, ApoA2 protein and ApoE protein, and the detection antibody is directed to an epitope present on a protein set forth in Table 1, wherein the capture and detection antibodies are directed to different epitopes.

Numerous embodiments (preferred and otherwise) are set forth above in connection with the instant methods and kits. Each embodiment explicitly set forth for any of the instant methods or kits applies, mutatis mutandis, to each of the other instant methods and kits, unless stated otherwise.

This invention will be better understood by reference to the examples which follow, but those skilled in the art will readily appreciate that the specific examples detailed are only illustrative of the invention as described more fully in the claims which follow thereafter.

### EXAMPLES

### Example 1

This invention provides a method of determining a mammalian test subject's risk of developing CVD by measuring apoA1 with a collection of antibodies, where each paratope is distinct, whose epitopes are distinguishable and interact in both conformation-dependent and -independent manner. The measurements from the subject's sample are then compared to one or more predetermined values measured in a control population of healthy subjects and to a collection of samples representing specific disorders.

Rationale: The lipoprotein apoA1 is the major constituent protein of HDL, accounting for 60-70% of the protein mass. Each particle is wrapped in 2-5 apoA1 proteins depending upon the size of the particle and the lipid composition (McLachlan, A. D., Nature, 267:465, 1977; Wu, Z., et. al., Nat. Struct. Mol. Biol. 14:861, 2007; Huang, R., et. al., Nat. Struct. Mol. Biol., Online 13 Mar. 2011). As the volume of the sphere changes with the gain or loss of lipid molecules, so will the particle diameter and circumference. As a consequence, the apoA1 molecules surrounding the lipid particle are also changing conformation to accommodate varying sphere geometries. Antibodies that recognize conformation-independent epitopes should have a greater probability of binding all apoA1 regardless of particle size. Analyzing serum with a panel of these antibodies should provide a measure of total apoA1 in the sample using a plurality of independent measurements and limits the risk of omission that a single antibody pair will produce. Total apoA1, whose level is predictive of CVD, can be used as a surrogate marker for the entire HDL population. Antibodies that interact with apoA1 in a conformation-dependent manner will recognize only those subsets of particles wherein apoA1 adopts the conformation recognized by that distinct paratope.

Measurements based upon a panel of these antibodies will identify HDL subpopulations based upon selected antibody pair values, whose levels vary between individual samples in a disease-specific manner. In one aspect, the present invention provides a diagnostic test in instances where paratope-epitope interactions are not yet defined.

Methods: A serum sample from an individual and those of a predetermined disease phenotype are subjected to a panel of capture-detection antibody pairs as defined in Table 3. Each of the 37 anti-apoA1 mAbs is evaluated for both its ability to work as a capture antibody and to act as a detection antibody. The total possible number of measurements is 1332 if the same antibody is not used for both capture and detection. Measurements are deemed positive if the positive signal is concentration dependent, saturable, reproducible and exhibits a linear response over a physiologically plausible range of concentration of apoA1.

Results: Antibody pairs demonstrating specific and saturable signals in a dose-dependent manner provide a measure of an existing HDL particle population present in the sample at concentrations that exceed the lowest level of detection that antibody pair affords. For all capture-detection antibody pairs resulting in a signal, analysis can be performed. Each antibody pair signal value can be statistically compared to itself and each other across a library of control samples and samples of known disease conditions. A select set of measurements showing strong correlations to each other across a sample set may represent a plurality of apoA1 epitopes associated with the same or highly similar particle subpopulation. Antibody pair signals that do not correlate with one another may be representative of independent particle subpopulations. Signals demonstrating the least variability across similar samples and the greatest variability between disease states are preferable for establishing predictive biomarkers of CVD. Each antibody pair signal value can be correlated to the surrogate marker total HDL-C surrogate level of a serum sample. Antibody pair signals having significant correlation to HDL-C are representative of subpopulations associated with large cholesterol-rich particles including the HDL2 particle fraction. Antibody pair signals having the least correlation with HDL-C levels are representative of small dense lipid poor HDL3 particle fraction which remains unaccounted for in the total HDL-C number. The greater the discordance between HDL-C levels and antibody pair signals, the more probable that the antibody pair is measuring an HDL subpopulation whose contributions to the HDL profile are not captured in the surrogate marker HDL-C.

### Example 2

This invention provides a method of determining a mammalian test subject's risk of developing CVD by measuring apoA2 with a collection of antibodies, where each paratope is distinct, whose epitopes are distinguishable and interact in both conformation-dependent and -independent manner. The measurements from the subject sample are then compared to one or more predetermined values measured in a control population of healthy subjects and to a collection samples representing specific disorders.

Rationale: The lipoprotein apoA2 is the second most abundant protein on HDL and is found in plasma as a monomer, homodimer, or heterodimer with apolipoprotein D (Schmitz, G., et. al., J. Lipid Res., 24:1021, 1983; Yang, C. Y., et al., Biochem. 33:12451, 1994; Gillard, B. K., et al. Biochem. 44:471, 2005). The differential equilibrium distribution between apoA1 and apoA2 across HDL2 and HDL3 sub-fractions has long been recognized (Cheung, M. C. and Albers, J. J., J. Lip. Res. 23:747, 1982). Most apoA2 is present on apoA1-containing particles and structural studies indicate that apoA2 can cause significant structural changes in apoA1 conformation, affecting both particle remodeling and activity (Rye, K. A. et. al., J. Biol. Chem., 278:22530, 2003; Boucher, J. et al., J. Lipid. Res. 45:849, 2004). In the plasma, apoA2 is associated predominantly with smaller and less lipid-enriched HDL particles. The denser HDL3 fraction has been shown to contain higher relative amounts of apoA2 than the larger HDL2 particles with apoA1/apoA2 ratios of 3.7 and 4.8, respectively (Brewer, H. B., Jr., et. al., Methods Enzymol. 128:223, 1986). The small dense fraction HDL3 demonstrates superior atheroprotective activities when compared to HDL2 isolated from the same individuals (Zerrad-Saadi, A., et. al., Arterioscler. Thromb. Vasc. Biol., 29:2169, 2009; Kontush, A., et. al., Arterioscler. Thromb. Vasc. Biol. 27:1843, 2007; de Souza, J. A., et. al., J. Cell. Mol. Med. 14:608, 2010), thus opening the possibility that individuals with low levels of HDL3 are thought to be at risk for CVD (see Kontush, A. and Chapman M. J. Nat. Clin Pract. 3:144, 2006).

Methods: A serum sample from an individual and those of a predetermined disease phenotype are subjected a panel of capture-detection antibody pairs directed at apoA2 from the list in Table 4. Each of the anti-apoA2 mAbs is evaluated both for its ability to work as a capture antibody and to act as a detection antibody. Measurements are deemed positive if the positive signal is concentration-dependent, saturable, reproducible and exhibits a linear response over a physiologically plausible range of apoA2 concentrations. Combining the panel of working apoA2 capture-detection antibody pairs and the antibody pairs identified as successfully generating a signal in Example 1 yields a mixed antigen measurement where one apoA1 and one apoA2 are used as capture-detection antibody pairs. As in Example 1, each apoA1|apoA2 mixed antigen antibody pair signal value can be statistically compared to itself and others across a library of control samples and samples of known disease condition.

Results: Antibody pairs demonstrating specific and saturable signals in a dose-dependent manner provide a means of measuring an existing particle population present in the sample at concentrations that exceed the lowest level of detection that antibody pair affords. For all mixed antigen capture-detection pairs resulting in a signal, analysis can be performed. Measurements from apoA1|apoA2 paired antibodies should identify HDL subpopulations containing both proteins. Antibody paired signal demonstrating the least variability across similar samples and the greatest variability between disease states are preferable for establishing predictive biomarkers of CVD. The measurements from the subject sample are then compared to one or more predetermined values measured in a control population of healthy subjects and to a collection of control and disease samples.

### Example 3

This invention provides a method of determining a mammalian test subject's risk of developing CVD through measuring a plurality of epitopes from the HDL proteome members defined by Table 1 using a selection of HDL proteome member antibodies from Table 4. Each antibody that binds a distinct proteome member can be used in mixed antigen capture-detection pairs with apoA1 or apoA2 if epitopes are distinguishable and non-overlapping. The measurements from the subject sample are then compared to one or more predetermined values measured in a control population of healthy subjects and to a collection samples representing specific disorders.

Rationale: Both apoA1 and apoA2 are present on the majority of HDL particles. Other proteome members demonstrate degrees of particle restriction and selectivity (Davidson, W. S, et. al. Arterioscler. Thromb. Vasc. Biol. 29:870, 2009; Gordon, S. M., et. al., J. Proteome Res., 9:5239, 2010).

Using a conformational-independent apoA1 capture antibody from Example 1 paired with a detection antibody for another proteome member can provide a measure of the relative concentration of the subpopulation containing both proteins in the sample. Both of the HDL particle populations, those containing the proteome member and those that do not, compete for binding to the same apoA1 capture antibody resulting in diminished signal. In other instances, proteome member capture antibodies will selectively bind only particle subpopulations containing the proteome member, and can preferentially concentrate particles containing the proteome member. Such a combination can increase the lower limits of detection in instances where the subpopulation defined by the proteome member is small. Antibody pair signals generated by measuring apoA1|proteome or proteome|apoA1 are different and can be statistically compared to themselves and others across a library of control samples and samples of known disease condition.

Methods: A serum sample from an individual and those of a predetermined disease phenotype are subjected to a panel of capture-detection antibody pairs derived from pairs successfully generating a signal in Example 1 or 2 with proteome-specific antibodies from Table 4. Each proteome mAb is evaluated both for its ability to work as a capture antibody and to act as a detection antibody. Measurements are deemed positive if the positive signal is concentration-dependent, saturable, reproducible and exhibits a linear response over a range of HDL concentrations. Mixed antigen antibody pair signal values can be statistically compared to themselves and other signals across a library of control samples and samples of known disease condition.

Results: Antibody pairs demonstrating specific and saturable signals in a dose-dependent manner provide a means of measuring an existing particle population present in the sample at concentrations that exceed the lowest level of detection that an antibody pair affords. For all mixed antigen capture-detection pairs resulting in a signal, analysis can be performed. Measurements from apoA1|proteome and proteome|apoA1 paired antibodies should identify HDL subpopulations containing both proteins. Antibody paired signals demonstrating the least variability across similar samples and the greatest variability between disease states are preferable for establishing predictive biomarkers of CVD.

### Example 4

This invention provides a method of determining a mammalian test subject's risk of developing CVD by measuring specific apoA1 conformations associated with levels of functional HDL subpopulations previously identified by physiochemical properties. The measurements from the subject sample are then compared to one or more predetermined values measured in a control population of healthy subjects and to a collection of samples representing specific disorders.

Rationale: Small lipid-poor preβ1-HDL, a minor HDL sub-fraction consisting of a discoidal-shaped particle containing apoA1, PL and unesterified cholesterol, can be identified by 2-D gel electrophoresis (Asztalos, B. F., et al., Arterioscler. Thromb. Vasc. Biol. 20:2670, 2000). Preβ1-HDL is a preferred acceptor of cellular cholesterol, an important first step in the process of reverse-cholesterol transport (Castro, G. R. and Fielding P. E., Biochemistry 27:25, 1988; Kawano, M., et. al., Biochem. 32:5025, 1993; Huang, Y., et. al., Arterioscler. Thromb. Vasc. Biol. 13:445, 1993). Levels of preβ1-HDL are elevated in type 2 diabetes and indicative of patients with hyperlipidemia and CAD (Hirayama, S., et. al., Diabetes Care 30:1289, 2007; Miida, T., et. al., Clin. Chem. 42:1992, 1996; Asztalos, B. F., et al., Arterioscler. Thromb. Vasc. Biol. 20:2670, 2000). Rather than characterizing preβ1-HDL using physiochemical separation, the particle population can be measured using a capture antibody highly specific for an apoA1 conformation found only in preβ1-HDL paired with a conformational-independent apoA1 detection antibody identified from Example 1. Such conformation-dependent antibodies include mAb 55201 (Miyazaki, O, et. al., J. Lipid Res., 41:2083) that recognizes an apoA1 epitope located between residues 140-210 (Sviridov, D., et. al., Arterioscler. Thromb. Vasc. Biol. 22:1482, 2002) or the mAb that recognizes apoA1 residues 137-144 of the mature protein uniquely associated with preβ1-HDL (Fielding, P. E., et. al., Biochemistry 33:6981, 1994). Elevated levels of preβ1-HDL are a predictor of carotid atherosclerosis (Suzuki, M. et. al., Clin. Chem. 51:132, 2005; Hirayama, S., et al., Diabetes Care 30:1289, 2007; Tashiro, J., et. al., Atherosclerosis 204:595, 2009).

Methods: A serum sample from an individual and those of a predetermined disease phenotype are measured using a specific conformation-dependent antibody capable of recognizing apoA1 only when present in the preβ1-HDL subpopulation, paired with a conformation-independent apoA1 detection antibody identified from Example 1. Measurements are deemed positive if the positive signal is concentration-dependent, saturable, reproducible and exhibits a linear response over a range of HDL concentrations. Mixed antigen antibody pair signal values can be statistically compared to themselves and other signals across a library of control samples and samples of known disease condition.

Results: Individuals with normal ranges of total HDL-C and total apoA1 levels can exhibit increased levels of preβ1-HDL subpopulation. Individuals with increased levels of preβ1-HDL are at risk for CAD and may also have compounding factors including dyslipidemia or diabetes.

### Example 5

In one embodiment, the present invention provides a method of determining a mammalian test subject's levels of functionally defective HDL particles resulting from specific post-translational modifications of apoA1. The measurements from the subject sample are compared to one or more predetermined values measured in a control population of healthy subjects and to a collection samples representing specific disorders.

Rationale: Post-translation modifications to apoA1 can result in functionally defective. HDL (see Kontush, A. and Chapman, M. J., Pharmacol. Rev., 58:342, 2006). Oxidized lipids and proteins associated with lipoprotein particles play a key role in atherogenesis (Barter, P. J., et. al., Circ. Res., 95:764, 2004; Nicholls, S. J., et. al., Trends Card. Med., 15:212, 2005; Deigner, H. P. and Hermetter, A., Curr. Opin. Lipidol., 19:289, 2008). Myeloperoxidase modifies apoA1 at specific susceptible residues (Met86, Met112, Met148, and Tyr192) resulting in functionally defective HDL (Zheng, L. B., et. al., J. Clin. Invest. 114:529, 2004; Shao, B. G., et. al., Proc. Natl. Acad. Sci. USA 105:12224, 2008; Shao, B., et. al., Chem. Res. Toxicol. 23(3):447, 2010). Antibodies developed to detect modification to those residues, MOA-I and mAb17 (Wang, X. S., et. al., J. Lipid Res. 50:586, 2009) can be employed to measure the extent of oxidated apoA1 in HDL when paired with an apoA1 capture antibody with a distinguishable and non-overlapping epitope as identified in Example 1. Another example of post-translational modification affecting HDL particle function is glycation (non-enzymatic glycosylation) which is the result of the bonding of a sugar molecule (fructose, glucose or galactose) with a protein or lipid molecule. Glycation is considered an arbitrary process which differs from glycosylation which involves enzyme-controlled addition of sugars to protein or lipid molecules at defined sites. Glycation can impair the functioning of biomolecules and this specific modification of apoA1 results in impaired anti-inflammatory activities of HDL (Calvo, C., et. al., Clin. Chinn. Acta, 217:193, 1993; Nobécourt, E., et. al., Arterioscler. Thromb. Vasc. Biol., 30:766, 2010; Park, K-H. and Cho, K-H., J. Gerontol., 66A:511, 2011). Methodology devised to generate specific antibodies capable of detecting specific glycation modified proteins can be employed to develop similar measure for glycanated apoA1 (Steward, L. A., et. al., J. Immuno. Method., 140:145, 1991; Cohen, M. P., et. al., Eur. J. Clin. Chem. Clin. Biochem., 31:707, 1993; Qin, X., et. al., Diabetes, 53:2653, 2004). In another example, secreted apoA1 exists as two species in plasma, a pro-protein and mature protein form which differ by six amino acid residues on the N-terminal end of the protein (Zannis, V. I., et. al., Proc. Natl. Acad. Sci. USA, 80:2574, 1983; Stoffel, W., J. Lipid Res., 25:1586, 1984).

### Example 6

This invention provides a method of determining the effects of apoC3 levels on a subject's risk for the disorders hypertriglyceridemia and CVD. The measurements from the subject sample are compared to one or more predetermined values measured in a control population of healthy subjects and to a collection samples representing specific disorders.

Rationale: Human apoC3 is a protein constituent of both apoB-containing lipoproteins and HDL (Shin, M. J. and Krauss R. M., Atherosclerosis 211:337, 2010). In addition to rapid transfer between particles, apoC3 redistributes from triglyceride-rich lipoproteins (TRLs) to HDL and transfers back to newly synthesized TRLs (see Jong, M. C., et al., Arterioscler. Thromb. Vasc. Biol., 19:472, 1999; Ooi, E. M. M., Clinical Science, 114:611, 2008). Through a genome-wide association study, APOC3 null mutation carriers were identified who had reduced apoC3 levels and had lower fasting triglycerides and postprandial serum triglycerides and increased HDL-C. Consistent with the favorable protective lipid profile, APOC3 null mutation carriers were less likely to have detectable coronary artery calcification (Pollin, T. I., et al., Science 322:1702, 2008). To test a subject for risks associated with dyslipidemia due to apoC3 disequilibrium, the combination of capture-detection antibody pairs can be used to measure the levels and disposition of apoC3 in a biological sample. The signal associated with these measurements in the test subject is compared to a predetermined value to determine if the subject is at greater risk of developing or suffering from CAD than subjects with an amount of apoC3 that is at, or higher than, the predetermined value. Moreover, the extent of the difference between the test subject's apoA1|apoC3 and apoA2|apoC3 levels in the biological sample and the predetermined value is also useful for characterizing the extent of the risk, and thereby determining which subjects would most greatly benefit from certain TG-lowering therapies.

### Example 7

This invention provides a method of determining the level of one or more lipoprotein proteome members selected from apoJ, PON1, PON3 and PAF-AH, as a method of assessing HDL subpopulations containing anti-oxidative activity. The measurements from the subject sample are compared to one or more predetermined values measured in a control population of healthy subjects and to a collection of samples representing specific disorders.

### Example 8

This invention provides a method of determining the level of one or more lipoprotein proteome members associated with HDL selected from AHSG, A1BG, apoF, GC, PLTP, RBP4, serpinA3, serpinA8, serpinF2 and TTR. The detected amount of the lipoprotein proteome member is compared to one or more predetermined values of the lipoprotein proteome member(s) measured in a control population of healthy subjects to evaluate the level of small dense HDL3. The measurements from the subject sample are then compared to one or more predetermined values measured in a control population of healthy subjects and to a collection samples representing specific disorders.

### Example 9

This invention provides methods of screening a human subject who appears healthy, or may be diagnosed as having a low HDL:LDL ratio and/or as being at risk for CVD based on certain known risk factors such as high blood pressure, high cholesterol, obesity, or genetic predisposition for CVD. The methods described herein are especially useful to identify subjects at high risk of developing CVD, in order to determine what type of therapy is most suitable and to avoid potential side effects due to the use of medications in low risk subjects. For example, prophylactic therapy is useful for subjects at some risk for CVD, including a low fat diet and exercise. For those at higher risk, a number of drugs may be prescribed by physicians, such as lipid-lowering medications as well as medications to lower blood pressure in hypertensive patients. For subjects at high risk, more aggressive therapy may be indicated, such as administration of multiple medications.

Envisioned here is a method of detecting arterial disease, atherosclerosis, and fatty lesions formed on the inside of the arterial wall. These lesions promote the loss of arterial flexibility and lead to the formation of blood clots. The lesions may also lead to thrombosis, resulting in most acute coronary syndromes. Thrombosis results from weakening of the fibrous cap, and thrombogenicity of the lipid core. It is well recognized that atherosclerosis is a chronic inflammatory disorder (see Ross, R., N. Engl. J. Med. 340:115, 1999). Chronic inflammation alters the protein composition of HDL, making it atherogenic (see Barter, P. J., et al., Circ. Res. 95:764, 2004; Chait, A., et al., J. Lipid Res. 46:389, 2005; Navab, M., et al., J. Lipid Res. 45:993, 2004; and Ansell, B. J., et al., Circulation 108:2751, 2003). Accordingly, HDL-associated proteins that serve as lipoprotein proteome member indicators for CVD, and atherosclerotic lesions in particular, may be derived from macrophages, smooth muscle cells, and endothelial cells present in atherosclerotic lesions. Accordingly, HDL-associated lipoprotein proteome members isolated from a blood sample represent a biochemical “biopsy” of the artery wall or endothelium lining the vasculature. It is likely that lesions that are most prone to rupture would increase their output of HDL due to the fact that enhanced proteolytic activity destroys the extracellular matrix and promotes plaque rupture. Indeed, short-term infusion of HDL into humans may promote lesion regression (Nissen, S. E., et al., JAMA 290:2292, 2003), suggesting that HDL can remove components of atherosclerotic tissue. Therefore, the proteins included in the protein cargo associated with HDL, enriched in CVD subjects, and also known to be present in lesion HDL from a population of CVD patients, serve as lipoprotein proteome members that may be used to detect the risk and/or presence of atherosclerotic plaques in an individual subject.

In another aspect, this invention provides assays comprising one or more detection reagents capable of detecting at least the proximity of two lipoprotein proteome members that is indicative of the presence or risk of CVD in a subject. The lipoprotein proteome member is detected by mixing a detection reagent that detects at least one lipoprotein proteome member associated with CVD with a sample containing HDL-associated proteins, and monitoring the mixture for detection of the lipoprotein proteome member with a suitable detection method such as spectrometry, immunoassay, or other method. In one example, the assays are provided as a kit. The kit can have, for example, detection reagents for detecting at least two, three, four, five, ten or more HDL-associated lipoprotein proteome members in biological samples from a test subject.

The kit also includes written indicia, such as instructions or other printed material for characterizing the risk of CVD based upon the outcome of the assay. The written indicia may include reference information, or a link to information regarding the predetermined signal values for paired proteome measurements of one, two, three, four, five, ten or more HDL-associated lipoprotein proteome members from a reference population of healthy subject samples, and an indication of a correlation between paired proteome measurements of one or more HDL-associated lipoprotein proteome members with samples from subjects having, or at risk of having, CVD.

In one example, the detection reagent comprises one or more antibodies which specifically bind one or more of the lipoprotein proteome members provided in Table 1 or 2 that may be used for the diagnosis and/or prognosis of CVD characterized by the relative abundance of the lipoprotein proteome member in the serum, or an HDL subfraction thereof. Standard values for protein levels of the lipoprotein proteome members are established by combining biological samples taken from healthy subjects. Deviation in the amount of signal produced from an antibody pair between control subjects and CVD subjects establishes the parameters for diagnosing and/or assessing risk levels, or monitoring disease progression.

In another example, this invention provides a method of determining the efficacy of a treatment regimen for treating and/or preventing CVD by monitoring the presence of one or more lipoprotein proteome members in a subject during treatment for CVD. The treatment for CVD varies depending on the symptoms and disease progression. The general treatments include lifestyle changes and medications, and may include surgery. Lifestyle changes include, for example, weight loss, a low saturated fat, low cholesterol diet, reduction of sodium, regular exercise, and a prohibition on smoking. Medications useful to treat CVD include, for example, cholesterol-lowering medications, antiplatelet agents (e.g., aspirin, ticlopidine and clopidogrel), glycoprotein IIb-IIIa inhibitors (such as abciximab, eptifibatide or tirofiban), or antithrombin drugs (blood-thinners such as heparin) to reduce the risk of blood clots. Beta-blockers may be used to decrease the heart rate and lower oxygen use by the heart. Nitrates, such as nitroglycerin are used to dilate the coronary arteries and improve blood supply to the heart. Calcium-channel blockers are used to relax the coronary arteries and systemic arteries, and, thus, reduce the workload for the heart. Medications suitable for reducing blood pressure are also useful to treat CVD, including ACE inhibitors, diuretics and other medical treatments.

TABLE 1

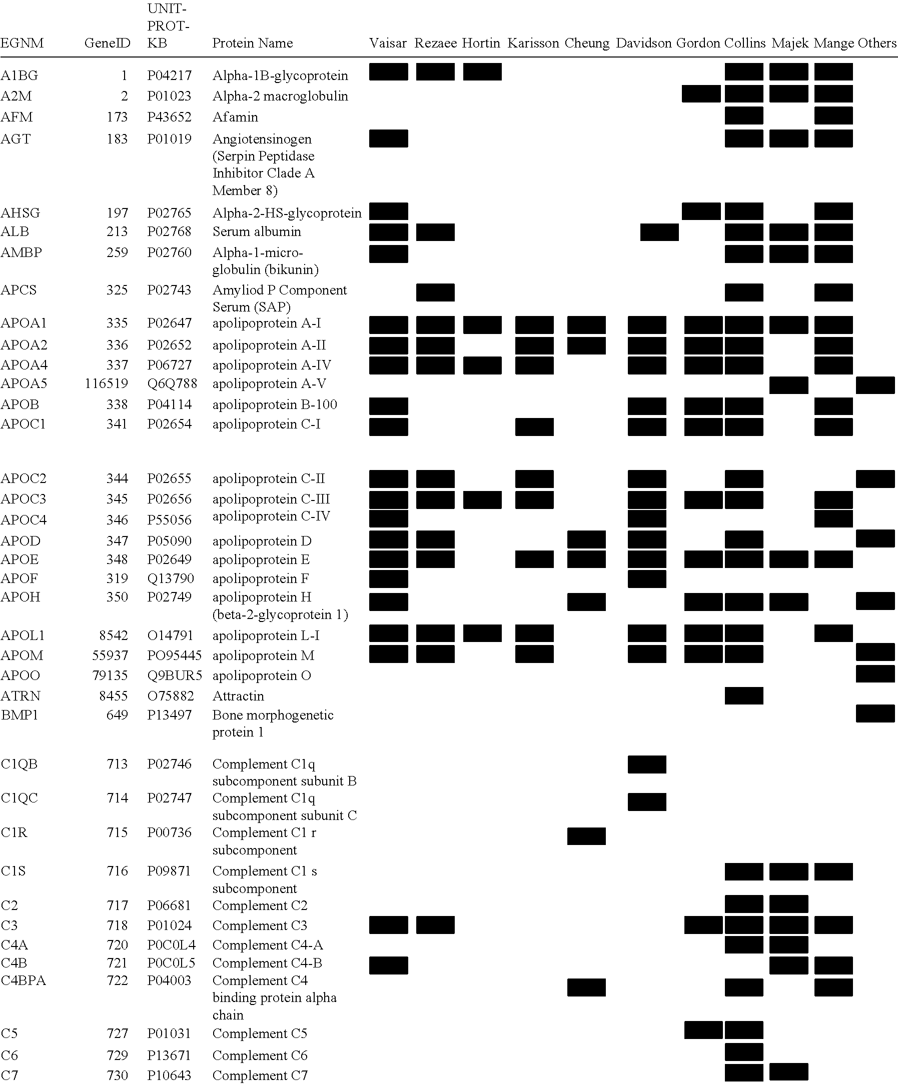

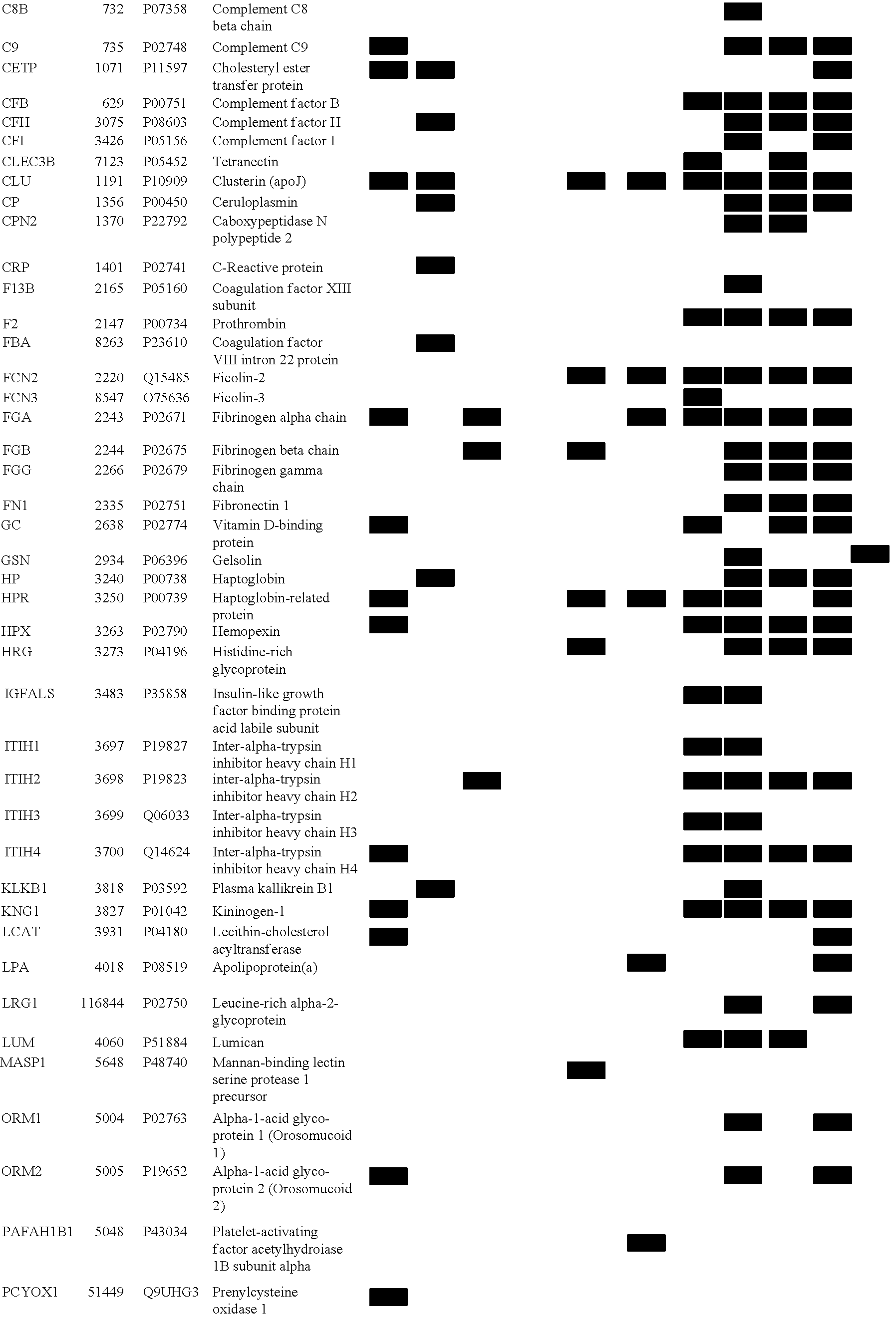

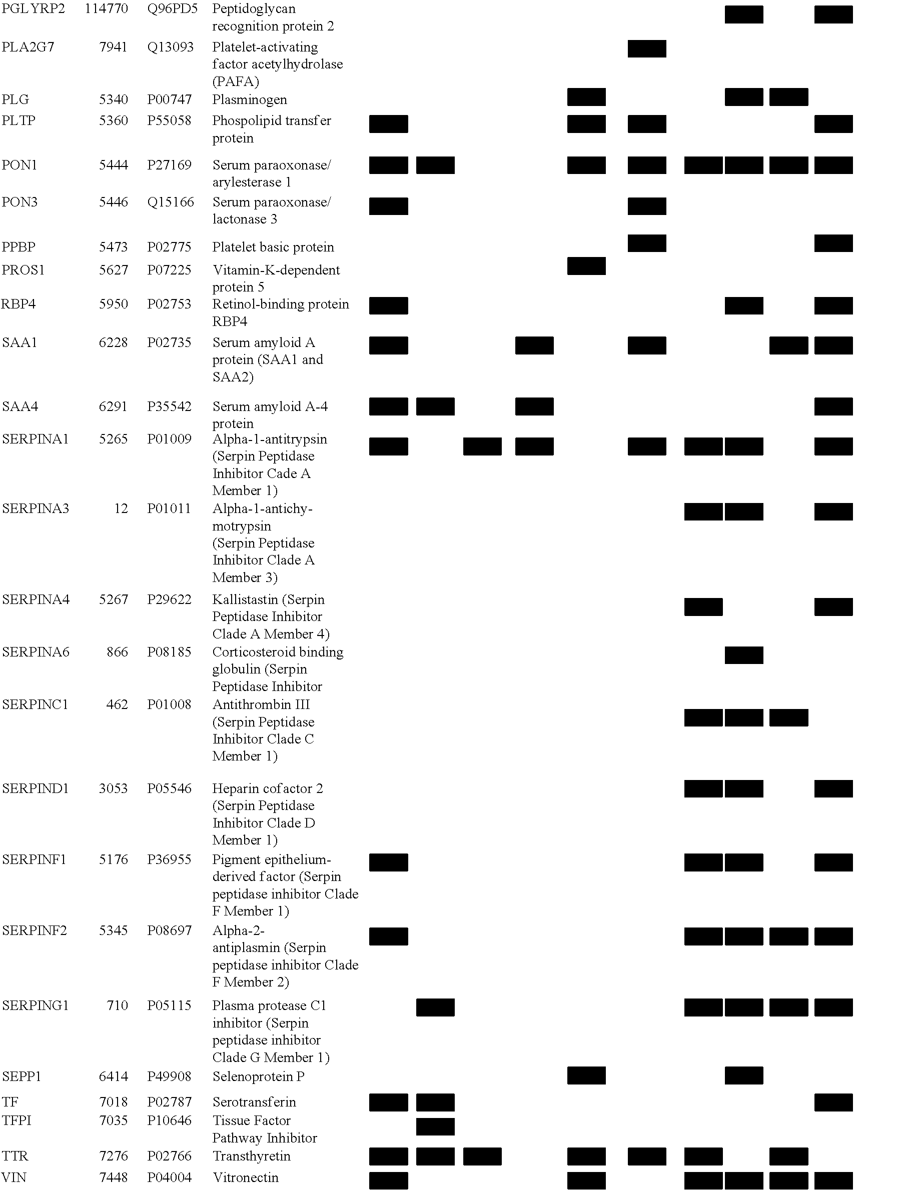

Vaisar, T., et. al., J. Clin. Invest., 177: 746, 2007
Rezaee, F., et. al., Proteomics 6: 721, 2006
Hortin, G. L., et. al., Biochem. Biophys. Res. Commun. 340: 909, 2006
Karlsson, H., et. al., Proteomics 5: 1431, 2005
Cheung, M. C., et. al., Biochem. 49: 7314, 2010
Davidson, W. S., et. al. ATVB 29: 870, 2009
Gordon, S. M., et. al., J. Proteome Res., 9: 5239, 2010
Collins, L. A. and Olivier, M., Proteome Sciences 4: 42, 2010
Lamant, M., et. al., J. Biol. Chem., 281: 36289, 2006
O'Brien, P. J. et. al., Clin Chem., 51: 351, 2005
Majek, P. et. al., J. Translational Medicine 9: 84, 2011
Mange, A., et. al., PLoS One 7: e34107, 2012

TABLE 2

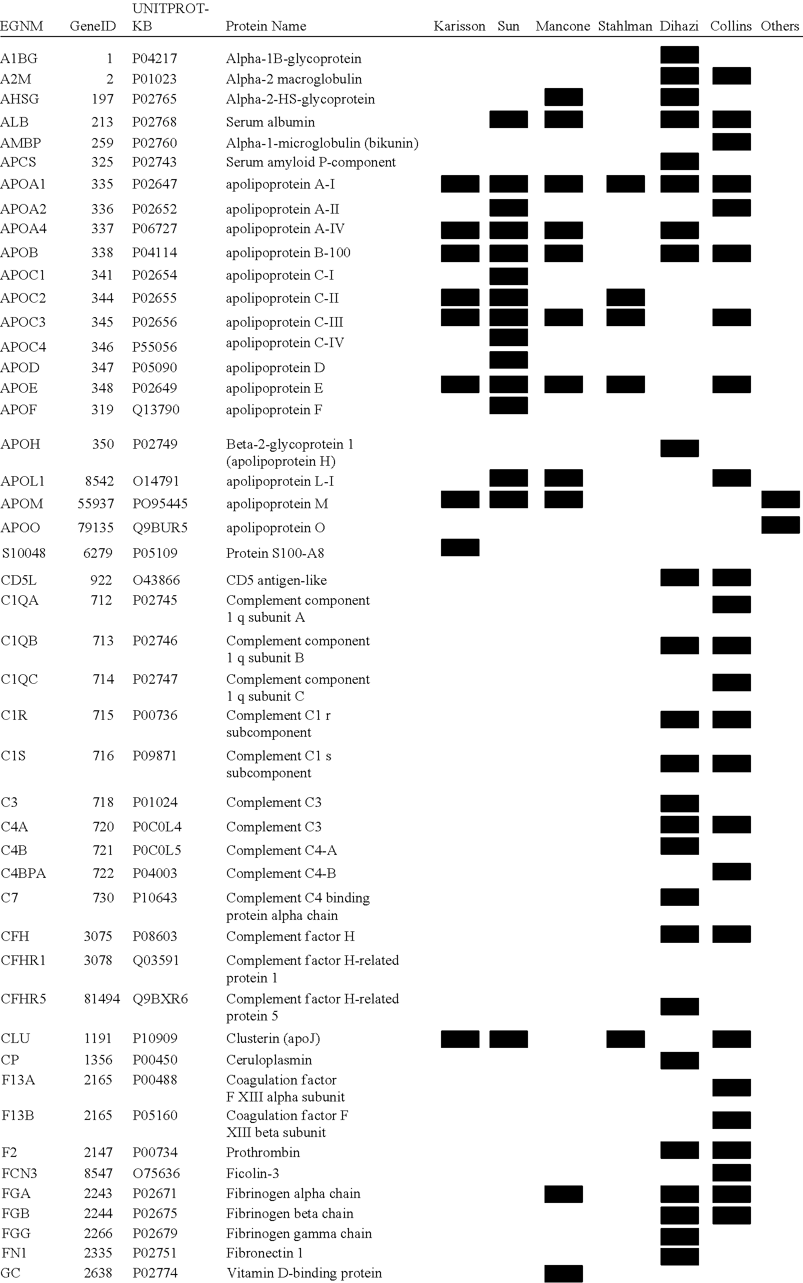

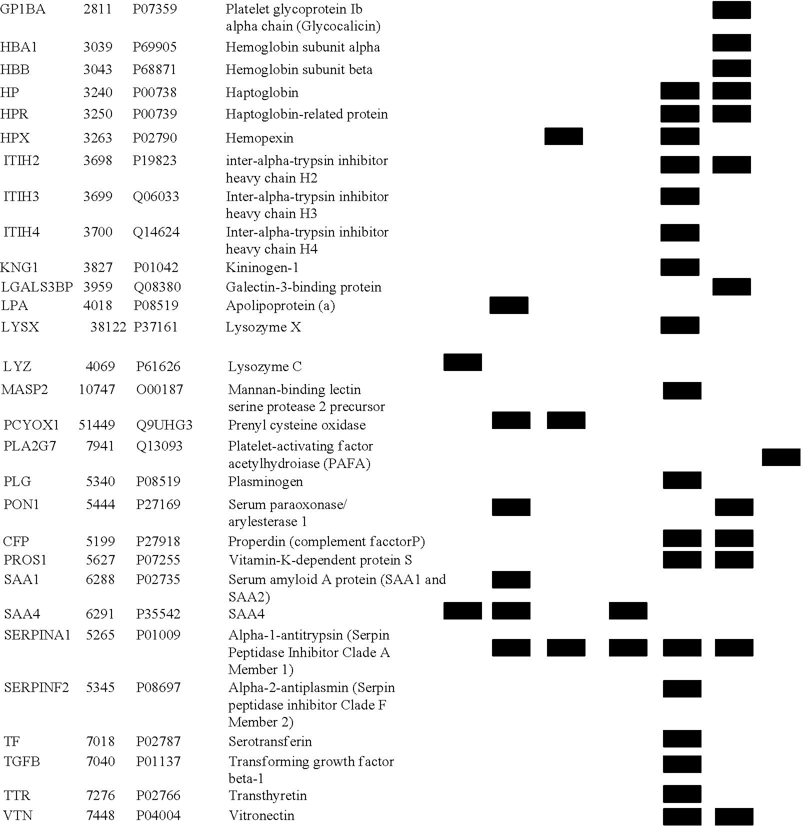

Karlsson, H., et. al., Proteomics 5: 551, 2005
Sun, H-Y, et. at., Clinica Chimica Acta 411: 336, 2010
Mancone, C., et. al., Proteomics 7: 143, 2007
Stahlman, M., et. al., J. Lipid Res. 49: 481, 2008
Dihazi, H., et. al., Nephrol. Dial. Transplant 23: 2925, 2008
Collins, L. A. and Olivier, M., Proteome Sciences 4: 42, 2010
Lamant, M., et. al., J. Biol. Chem., 281: 36289, 2006
Tew, D. G., et. al., Arterioscler. Thromb. Vasc. Biol. 16: 591, 1996

TABLE 3

| Clone ID | Host | Specificty | Isotype | Vendor | Cat. # |
|---|---|---|---|---|---|
| 1402 | mouse | human | IgG1 | Abcam | ab20411 |
| 1405 | mouse | human | IgG1 | Abcam | ab20735 |
| 1409 | mouse | human | IgG1 | Abcam | ab20918 |
| 1C5 | mouse | human | IgG1 | Biodesign Intl. | H61531M |
| 513 | mouse | human | IgG1 | CalBiochem | 178472 |
| 6001 | mouse | human | IgG2a | CalBiochem | 178470 |
| 412 | mouse | human | IgG1 | EMD Millipore | MAB010-A/11 |
| A/13 | mouse | human | IgG1 | EMD Millipore | MAB011-A/13 |
| EP1368Y | rabbit | human | n.d. | Epitomics | 1920-1 |
| LS-B3047 | mouse | human | IgG2a, kappa | LifeScience Bio | 057-10029 |
| LS-C84251 | mouse | human | IgG2a | LifeScience Bio | M55311 |
| LS-C84252 | mouse | human | IgG2a | LifeScience Bio | M808121 |
| LS-C35007 | mouse | human | IgG1 | LifeScience Bio | 1402 |
| LS-C35008 | mouse | human | IgG1 | LifeScience Bio | 1404 |
| HDL 110 | mouse | human | IgG2b | Mabtech | 3710-2-1000 |
| HDL 44 | mouse | human | IgG1 | Mabtech | 3710-3-1000 |
| 412 | mouse | human | IgG1 | Millipore | MAB010-A/11 |
| 057-10029 | mouse | human | IgG2a, kappa | MYBIOSOURCE | MBS311600 |
| 057-16001 | mouse | human | IgG2a, kappa | MYBIOSOURCE | MBS311599 |
| G2 | mouse | human | IgG1 | Novus Biologicals | NB100-65491 |
| 12C8 | mouse | human | IgG1, kappa | Novus Biologicals | NBP1-05174 |
| 2G4 | mouse | human | IgG1, kappa | Novus Biologicals | NBP1-41969 |
| 6A9G6 | mouse | human | IgG1 | ProMab | Mab-2008031-1 |
| 5F4F5 | mouse | human | IgG1 | ProMab | Mab-2008031-2 |
| A5.4 | mouse | human | IgG1 | Sant Cruz Biotechnology | sc-13549 |
| 3A11-1A9 | mouse | human | IgG2 | Sigma | WH0000335M1 |
| 2Q2200 | mouse | human | IgG1 | United States Biological | A2299-08C |
| 6F31 | mouse | human | IgG1, kappa | United States Biological | A2299-26A |
| 6F30 | mouse | human | IgG1, kappa | United States Biological | A2299-26 |
| 2Q2199 | mouse | human | IgG1 | United States Biological | A2299-08B |
| 5E12 | mouse | human | IgG2a, kappa | United States Biological | A2299-25A |
| 8.F.15 | mouse | human | IgG2a | United States Biological | A2299-12 |
| 2Q2201 | mouse | human | IgG1 | United States Biological | A2299-08D |
| 7K4 | mouse | human | IgG1 | United States Biological | A2299-09B |
| 4A89 | mouse | human | IgG2a, kappa | United States Biological | A2299-08F |
| 9L39 | mouse | human | IgG2a, kappa | United States Biological | A2299-09A1 |
| 10H10 | mouse | human | IgG1 | Yorkshire Bioscience | R1003 |
| 7C1 | mouse | human | IgG1 | Yorkshire Bioscience | R1005 |

TABLE 4

| EGNM | GeneID | UNIPROT-KB | Protein Name | Clone ID | Host | Specificty | Preparation | Quantity | | Vendor |
|---|---|---|---|---|---|---|---|---|---|---|
| A1BG | 1 | P04217 | Alpha-1B-glycoprotein | 4BS | mouse | human | ascites | 0.1 | mL | Novus Biologicals |
| A1BG | 1 | P04217 | Alpha-1B-glycoprotein | 1H1 | mouse | human | IgG purified | 100 | μg | Novus Biologicals |
| A1BG | 1 | P04217 | Alpha-1B-glycoprotein | 4F6 | mouse | human | IgG purified | 100 | μg | Novus Biologicals |
| A2M | 2 | P01023 | Alpha-2 macroglobulin | 1.B.742 | mouse | human | purified by DEAE chromatography. | 1 | mg | United States Biological |
| A2M | 2 | P01023 | Alpha-2 macroglobulin | 9L177 | mouse | human | purified by DEAE chromatography. | 1 | mg | United States Biological |
| A2M | 2 | P01023 | Alpha-2 macroglobulin | 9H154 | mouse | human | purified by Protein G affinity | 500 | μg | United States Biological |
| AFM | 173 | P43652 | Afamin | poly | mouse | human | n.d. | 50 | μg | Anitbodies-online.corn |
| AGT | 183 | P01019 | Angiotensinogen (Serpin Peptidase | BGN/KA/22H | mouse | human | affinity purified | 200 | μg | Novus Biologicals |
| | | | Inhibitor Clade A Member 8) | | | | | | | |
| AGT | 183 | P01019 | Angiotensinogen (Serpin Peptidase ) | 7H144 | mouse | human | purified by Protein G affinity | 100 | μg | United States Biological |
| | | | Inhibitor Clade A Member 8 | | | | | | | |
| AGT | 183 | P01019 | Angiotensinogen (Serpin Peptidase | 7C4 | mouse | human | purified | 50 | μg | United States Biological |
| | | | Inhibitor Clade A Member 8) | | | | | | | |
| AGT | 183 | P01019 | Angiotensin I | BGN/KA/4H | mouse | human | Protein A purified | 200 | μg | Novus Biologicals |
| AGT | 183 | P01019 | Angiotensin II | Ang II E7 | mouse | human | affinity purified | 200 | μg | Novus Biologicals |
| | | | | (BGN/KA/4L) | | | | | | |
| AGT | 183 | P01019 | Angiotensin II | BGN/0856/21 | mouse | human | Protein A purified | 200 | μg | Novus Biologicals |
| AGT | 183 | P01019 | Angiotensin II | 1A10 | mouse | human | IgG purified | 100 | μg | Novus Biologicals |
| AGT | 183 | P01019 | Angiotensin II | 3C10 | mouse | human | IgG purified | 100 | μg | Novus Biologicals |
| AHSG | 197 | P02765 | Alpha-2-HS-glycoprotein | 162919 | mouse | human | purified | 500 | μg | R & D Systems |
| AHSG | 197 | P02765 | Alpha-2-HS-glycoprotein | 162922 | mouse | human | purified | 500 | μg | R & D Systems |
| AHSG | 197 | P02765 | Alpha-2-HS-glycoprotein | 5D8 | mouse | human | IgG purified | 100 | μg | Novus Biologicals |
| AHSG | 197 | P02765 | Alpha-2-HS-glycoprotein | 1D8 | mouse | human | IgG purified | 100 | μg | Novus Biologicals |
| AHSG | 197 | P02765 | Alpha-2-HS-glycoprotein | 9H121 | mouse | human | purified by Protein G affinity | 500 | μg | United States Biological |
| ALB | 213 | P02768 | Serum albumin | 2F11 | mouse | human | purified by affinity chromatography | 0.1 | mL | Origene Technologies |
| ALB | 213 | P02768 | Serum albumin | 3F1 | mouse | human | purified by affinity chromatography | 0.1 | mL | Origene Technologies |
| ALB | 213 | P02768 | Serum albumin | 4D8 | mouse | human | purified by affinity chromatography | 0.1 | mL | OrigeneTechnologies |
| ALB | 213 | P02788 | Serum albumin | 4G9 | mouse | human | purified by affinity chromatography | 0.1 | mL | Origene Technologies |
| ALB | 213 | P02768 | Serum albumin | AL-01 | mouse | human | purified | 50 | μg | MyBiosource |
| ALB | 213 | P02768 | Serum albumin | M603208 | mouse | human | column chromatography purified | n.d. | | Lifespan Biosciences |
| ALB | 213 | P02768 | Serum albumin | A1-01 | mouse | human | purified | 100 | μg | United States Biological |
| ALB | 213 | P02768 | Serum albumin | BGN/1328/52 | mouse | human | purified IgG-liquid | 200 | μg | MyBiosource |
| ALB | 213 | P02768 | Serum albumin | 15C7 | mouse | human | purified, unlabeled | 1 | mg | MyBiosource |
| ALB | 213 | P02768 | Serum albumin | 1A9 | mouse | human | purified, unlabeled | 1 | mg | MyBiosource |
| ALB | 213 | P02768 | Serum albumin | 1C8 | mouse | human | purified, unlabeled | 1 | mg | MyBiosource |
| ALB | 213 | P02768 | Serum albumin | 6B11 | mouse | human | purified, unlabeled | 1 | mg | MyBiosource |
| ALB | 213 | P02768 | Serum albumin | 5B11 | mouse | human | n.d. | 200 | μg | Thermo Scientific Pierce |
| ALB | 213 | P02768 | Serum albumin | 15D2 | mouse | human | n.d. | 200 | μg | Thermo Scientific Pierce |
| ALB | 213 | P02768 | Serum albumin | 6B7 | mouse | human | n.d. | 200 | μg | Thermo Scientific Pierce |
| ALB | 213 | P02768 | Serum albumin | 1G2 | mouse | human | n.d. | 100 | μg | Thermo Scientific Pierce |
| ALB | 213 | P02768 | Serum albumin | AL-01 | mouse | human | n.d. | 100 | μg | Thermo Scientific Pierce |
| ALB | 213 | P02768 | Serum albumin | 2B3 | mouse | human | n.d. | 500 | μg | Thermo Scientific Pierce |
| ALB | 213 | P02768 | Serum albumin | 2B2 | mouse | human | n.d. | 500 | μg | Thermo Scientific Pierce |
| ALB | 213 | P02768 | Serum albumin | 2B6 | mouse | human | n.d. | 500 | μg | Thermo Scientific Pierce |
| ALB | 213 | P02768 | Serum albumin | KT11 | mouse | human | n.d. | 100 | μg | Thermo Scientific Pierce |
| ALB | 213 | P02768 | Serum albumin | KT32 | mouse | human | n.d. | 100 | μg | Thermo Scientific Pierce |
| ALB | 213 | P02768 | Serum albumin | 5D5 | mouse | human | n.d. | 200 | μg | Thermo Scientific Pierce |
| ALB | 213 | P02768 | Serum albumin | AL-01 | mouse | human | biotin conjugate | 100 | μg | Novus Biologicals |
| AMBP | 259 | P02760 | Alpha-1-microglobulin (bikunin) | 10000 | mouse | human | IgG purified | 100 | μg | Novus Biologicals |
| AMBP | 259 | P02760 | Alpha-1-microglobulin (bikunin) | 20 | mouse | human | IgG purified | 100 | μg | Novus Biologicals |
| AMBP | 259 | P02760 | Alpha-1-microglobulin (bikunin) | 4A7 | mouse | human | IgG purified | 100 | μg | Novus Biologicals |
| AMBP | 259 | P02760 | Alpha-1-microglobulin (bikunin) | 4C10 | mouse | human | IgG purified | 100 | μg | Novus Biologicals |
| AMBP | 259 | P02760 | Alpha-1-microglobulin (bikunin) | 4F2 | mouse | human | IgG purified | 100 | μg | Novus Biologicals |
| AMBP | 259 | P02760 | Alpha-1-microglobulin (bikunin) | 4E4 | mouse | human | IgG purified | 100 | μg | Novus Biologicals |
| AMBP | 259 | P02760 | Alpha-1-microglobulin (bikunin) | 4F8 | mouse | human | IgG purified | 100 | μg | Novus Biologicals |
| AMBP | 259 | P02760 | Alpha-1-microglobulin (bikunin) | 4G12 | mouse | human | IgG purified | 100 | μg | Novus Biologicals |
| AMBP | 259 | P02760 | Alpha-1-microglobulin (bikunin) | 4G2 | mouse | human | IgG purified | 100 | μg | Novus Biologicals |
| AMBP | 259 | P02760 | Alpha-1-microglobulin (bikunin) | 1F7 | mouse | human | IgG purified | 100 | μg | Novus Biologicals |
| AMBP | 259 | P02760 | Alpha-1-microglobulin (bikunin) | 9i21 | mouse | human | purified | 100 | μg | MyBiosource |
| AMBP | 259 | P02760 | Alpha-1-microglobulin (bikunin) | 9i22 | mouse | human | purified | 100 | μg | MyBiosource |
| AMBP | 259 | P02760 | Alpha-1-microglobulin (bikunin) | 9i24 | mouse | human | purified | 100 | μg | MyBiosource |
| AMBP | 259 | P02760 | Alpha-1-microglobulin (bikunin) | 9i25 | mouse | human | purified | 100 | μg | MyBiosource |
| AMBP | 259 | P02760 | Alpha-1-microglobulin (bikunin) | 9i26 | mouse | human | purified | 100 | μg | MyBiosource |
| AMBP | 259 | P02760 | Alpha-1-microglobulin (bikunin) | 9i28 | mouse | human | purified | 100 | μg | MyBiosource |
| AMBP | 259 | P02760 | Alpha-1-microglobulin (bikunin) | 9i29 | mouse | human | purified | 100 | μg | MyBiosource |
| AMBP | 259 | P02760 | Alpha-1-microglobulin (bikunin) | 3F1 | mouse | human | IgG purified | 100 | μg | Novus Biologicals |
| APCS | 325 | P02743 | Amyloid P Component Serum (SAP) | 3H48 | mouse | human | purified by Protein A affinity | 50 | μg | United States Biological |
| APCS | 325 | P02743 | Amyloid P Component Serum (SAP) | 4F3 | mouse | human | IgG purified | 100 | μg | Novus Biologicals |
| APCS | 325 | P02743 | Amyloid P Component Serum (SAP) | 1DB161 | mouse | human | purified by Protein A affinity | 200 | μg | United States Biological |
| APCS | 325 | P02743 | Amyloid P Component Serum (SAP) | 3H48 | mouse | human | purified by Protein A affinity | 50 | μg | United States Biological |
| APCS | 325 | P02743 | Amyloid P Component Serum (SAP) | 5.4A | mouse | human | purified | 100 | μg | EMD Millipore |
| APCS | 325 | P02743 | Amyloid P Component Serum (SAP) | 5.4D.3B | mouse | human | purified | 100 | μg | EMD Millipore |
| APCS | 325 | P02743 | Amyloid P Component Serum (SAP) | 5G6 | mouse | human | culture supernatant | 500 | [symbol] l | United States Biological |
| APCS | 325 | P02743 | Amyloid P Component Serum (SAP) | 2Q2175 | mouse | human | purified by Protein A affinity | 100 | μg | United States Biological |
| APCS | 325 | P02743 | Amyloid P Component Serum (SAP) | 2Q2174 | mouse | human | purified by Protein A affinity | 100 | μg | United States Biological |
| APOA2 | 336 | P02652 | apolipoprotein A-II | 057-10221 | mouse | human | purified, unlabeled | 1 | mg | MyBiosource |
| APOA2 | 336 | P02652 | apolipoprotein A-II | 4F3 | mouse | human | IgG purified | 100 | μg | Novus Biologicals |
| APOA2 | 336 | P02652 | apolipoprotein A-II | 1H6 | mouse | human | IgG purified | 100 | μg | Novus Biologicals |
| APOA2 | 336 | P02652 | apolipoprotein A-II | 4A90 | mouse | human | purified by Protein A affinity | 1 | mg | United States Biological |
| APOA2 | 336 | P02652 | apolipoprotein A-II | 10B188 | mouse | human | purified by Protein A affinity | 100 | μg | United States Biological |
| APOA2 | 336 | P02652 | apolipoprotein A-II | 057-10221 | mouse | human | purified by Protein A affinity | 1 | mg | Antibodies-online.com |
| APOA2 | 336 | P02652 | apolipoprotein A-II | 7H205 | rat | human | purified by Protein G affinity | 100 | μg | United States Biological |
| APOA2 | 336 | P02652 | apolipoprotein A-II | EP2912 | rabbit | human | purified, unlabeled | 0.1 | mL | Origene |
| APOA2 | 336 | P02652 | apolipoprotein A-II | EP2913 | rabbit | human | purified, unlabeled | 0.1 | mL | Origene |
| APOA2 | 336 | P02652 | apolipoprotein A-II | 395906 | rat | human | purified by Protein G affinity | 100 | μg | R & D Systems |
| APOA4 | 337 | P06727 | apolipoprotein A-IV | 1D686,1D4C11 | mouse | human | ascitic fluid containing 0.03% sodium az | 0.1 | mL | ProMab Biotechnotogies |
| APOA4 | 337 | P06727 | apolipoprotein A-IV | 2D1C9 | mouse | human | ascitic fluid containing 0.03% sodium az | 0.1 | mL | ProMab Biotechnologies |
| APOA4 | 337 | P06727 | apolipoprotein A-IV | poly | mouse | human | purified | 50 | μg | United States Biological |
| APOA5 | 116519 | Q6Q788 | apolipoprotein A-V | 10C4S7 | mouse | human | purified | 100 | μg | MyBiosource |
| APOA5 | 116519 | Q6Q788 | apolipoprotein A-V | 2G1H11, 1F1E8 | mouse | human | purified antibody in PBS containing 0.03 | 100 μg × 2 | | ProMab Biotechnologies |
| APOA5 | 116519 | Q6Q788 | apolipoprotein A-V | 1G5G9 | mouse | human | n.d. | 0.1 | mL | Origene Technologies |
| APOA5 | 116519 | Q6Q788 | apolipoprotein A-V | 1F1E8 | mouse | human | purified, unlabeled | 100 | μg | MyBiosource |
| APOA5 | 116519 | Q6Q788 | apolipoprotein A-V | 2G1H11 | mouse | human | purified, unlabeled | 100 | μg | MyBiosource |
| APOA5 | 116519 | Q6Q788 | apolipoprotein A-V | 1F1E8 | mouse | human | n.d. | 100 | μg | immuno-Biological |
| | | | | | | | | | | Laboratories |
| APOA5 | 116519 | Q6Q788 | apolipoprotein A-V | 4H8H8E2 | mouse | human | n.d. | 0.1 | mL | Thermo Scientific Pierce |
| APOA5 | 116519 | Q6Q788 | apolipoprotein A-V | 1G5G9 | mouse | human | n.d. | 0.1 | mL | Thermo Scientific Pierce |
| APOA5 | 116519 | Q6Q788 | apolipoprotein A-V | 8J289 | mouse | human | purified by Protein G affinity | 0.1 | mL | United States Biological |
| APOA5 | 116519 | Q6Q788 | apolipoprotein A-V | 8J288 | mouse | human | purified by Protein G affinity | 0.1 | mL | United States Biological |
| APOA5 | 116519 | Q6Q788 | apolipoprotein A-V | 10C456 | mouse | human | immunoaffinity purified | 100 | μg | United States Biological |
| APOA5 | 116519 | Q6Q788 | apolipoprotein A-V | 10C457 | mouse | human | immunoaffinity purified | 100 | μg | United States Biological |
| APOA5 | 116519 | Q6Q788 | apolipoprotein A-V | 8A105 | mouse | human | ascites | 40 | μg, | United States Biological |
| | | | | | | | | 5 × 40 μ [symbol] | | |
| APOA5 | 116519 | Q6Q788 | apolipoprotein A-V | 4H8H8E2 | mouse | human | Protein G purified | 0.1 | mL | Novus Biologicals |
| APOA5 | 116519 | Q6Q788 | apolipoprotein A-V | 1G5G9 | mouse | human | Protein G purified | 0.1 | mL | Novus Biologicals |
| APOB | 338 | P04114 | apolipoprotein B-100 | 369717 | mouse | human | purified | 100 | μg | R & D Systems |
| APOB | 338 | P04114 | apolipoprotein B-100 | 10B190 | mouse | human | purified by DEAE chromatography. | 1 | mg | United States Biological |
| APOB | 338 | P04114 | apolipoprotein B-100 | 7H145 | mouse | human | purified by Protein G affinity | 100 | μg | United States Biological |
| APOB | 338 | P04114 | apolipoprotein B-100 | 369717 | mouse | human | purified | 100 | μg | R & D Systems |
| APOB | 338 | P04114 | apolipoprotein B-100 | F2C9 | mouse | human | purified IgG-liquid | 1 | mg | MyBiosource |
| | | | | (APOB-001) | | | | | | |
| APOB | 338 | P04114 | apolipoprotein B-100 | 10B190 | mouse | human | purified by DEAE chromatography. | 1 | mg | United States Biological |
| APOB | 338 | P04114 | apolipoprotein B-100 | C1.4 | mouse | human | purified | 200 | μg/ml | Santa Cruz Biotechnology |
| APOB | 338 | P04114 | apolipoprotein B-100 | APOB-001 | mouse | human | affinity purified | 1.0 | mg | Novus Biological |
| APOB | 338 | P04114 | apolipoprotein B-100 | LDL 11 | mouse | human | biotinylated, supplied at 1 mg/ml | 250 | μg | Mabtech AB |
| APOB | 338 | P04114 | apolipoprotein B-100 | LDL 20/17 | mouse | human | purified, supplied at 1 mg/ml | 250 | μg | Mabtech AB |
| APOB | 338 | P04114 | apolipoprotein B-100 | 5i2 | mouse | human | purified | 50 | μg | United States Biological |
| APOB | 338 | P04114 | apolipoprotein B-100 | 5F8 | mouse | human | n.d. | 1 | mL | Thermo Scientific Pierce |
| APOB | 338 | P04114 | apolipoprotein B-100 | 12G10 | mouse | human | n,d, | 1 | mL | Thermo Scientific Pierce |
| APOB | 338 | P04114 | apolipoprotein B-100 | 4C11 | mouse | human | n.d. | 1 | mL | Thermo Scientific Pierce |
| APOB | 338 | P04114 | apolipoprotein B-100 | 2000 | mouse | human | n.d. | 1 | mL | Thermo Scientific Pierce |
| APOB | 338 | P04114 | apolipoprotein B-100 | B1 | mouse | human | purified | 500 | μg | EMD Millipore |
| APOB | 338 | P04114 | apolipoprotein B-100 | 7B8 | mouse | human | n.d. | 200 | μg | Thermo Scientific Pierce |
| APOB | 338 | P04114 | apolipoprotein B-100 | 7C2 | mouse | human | n.d. | 200 | μg | Thermo Scientific Pierce |
| APOB | 338 | P04114 | apolipoprotein B-100 | 9G3 | mouse | human | n.d. | 200 | μg | Thermo Scientific Pierce |
| APOC1 | 341 | P02654 | apolipoprotein C-I | 2E2-1A3 | mouse | human | IgG purified | 100 | μg | Novus Biological |
| APOC1 | 341 | P02654 | apolipoprotein C-I | 10B191 | mouse | human | Purified by Protein A affinity | 100 | μg | United States Biological |
| APOC2 | 344 | P02655 | apolipoprotein C-II | | | | | | | |
| APOC3 | 345 | P02656 | apolipoprotein C-III | 2Q2198 | mouse | human | Ascites | 100 | μg | United States Biological |
| APOC4 | 346 | P55056 | apolipoprotein C-IV | 3D10 | mouse | human | IgG purified | 100 | μg | Novus Biologicals |
| APOD | 347 | P05090 | apolipoprotein D | 2B15 | mouse | human | purified by ammonium sulfate pre [symbol] | 0.1 | mL | United States Biological |
| APOD | 347 | P05090 | apolipoprotein D | 36C6 | mouse | human | supernatant | 0.2 | mL | BIOLOGO |
| APOE | 348 | P02649 | apolipoprotein E | WUE-4 | mouse | human | protein G purified | 0.1 | mL | Origene Technologies |
| APOE | 348 | P02649 | apolipoprotein E | A1.4 | mouse | human | purified | 200 | μg/ml | Santa Cruz Biotechnology |
| APOE | 348 | P02649 | apolipoprotein E | 40000 | mouse | human | Protein G purified | 0.1 | mL | Novus Biologicals |
| APOE | 348 | P02649 | apolipoprotein E | 5B5 | mouse | hu man | purified | 200 | μg | Immuno-Biological |
| | | | | | | | | | | Laboratories |
| APOE | 348 | P02649 | apolipoprotein E | E887 | mouse | human | biotinylated, supplied at 0.5 mg/ml | 250 | μg | Mabtech AB |
| APOE | 348 | P02649 | apolipoprotein E | 3D12 | mouse | human | purified, unconjugated | 100 | μg | YO Proteins AB |
| APOE | 348 | P02649 | apolipoprotein E | E3 | mouse | human | affinity purified, unconjugated | 0.2 | mL | Amrican Research Products |
| APOE | 348 | P02649 | apolipoprotein E | 1B5-E1 | mouse | human | affinity purified, unconjugated | 0.2 | mL | Amrican Research Products |
| APOE | 348 | P02649 | apolipoprotein E | F48.1 | mouse | human | affinity purified, unconjugated | 0.2 | mL | Amrican Research Products |
| APOE | 348 | P02649 | apolipoprotein E | 8B65 | mouse | human | purified by Protein G affinity | 0.1 | mL | United States Biological |
| APOE | 348 | P02649 | apolipoprotein E | 7C10 | mouse | human | purified by Protein A affinity | 50 | μg | United States Biological |
| APOE | 348 | P02649 | apolipoprotein E | 5F194 | mouse | human | purified by Protein G affinity | 50 | μg | United States Biological |
| APOE | 348 | P02649 | apolipoprotein E | 5F211 | mouse | human | purified by Protein G affinity | 50 | μg | United States Biological |
| APOE | 348 | P02649 | apolipoprotein E | E887 | mouse | human | biotinylated, supplied at 0.5 mg/ml | 250 | μg | Mabtech AB |
| APOE | 348 | P02649 | apolipoprotein E | E276 | mouse | human | purified, supplied at 0.5 mg/ml | 250 | μg | Mabtech AB |
| APOE | 348 | P02649 | apolipoprotein E | E6D7 | mouse | human | purified | 0.1 | mL | MyBiosource |
| APOE | 348 | P02649 | apolipoprotein E | 2B16 | mouse | human | purified by Protein A affinity | 0.1 | mL | United States Biological |
| APOF | 319 | Q13790 | apolipoprotein F | 1D5 | mouse | human | IgG purified | 100 | μg | Novus Biologicals |
| APOH | 350 | P02749 | apolipoprotein H (beta-2-glycoprotein 1) | 5-F7 | mouse | human | purified | 100 | μg | EMD Millipore |
| APOH | 350 | P02749 | apolipoprotein H (beta-2-glycoprotein 1) | 3F10 | mouse | human | IgG purified | 0.2 | mL | Novus Biologicals |
| APOH | 350 | P02749 | apolipoprotein H (beta-2-glycoprotein 1) | 517038 | mouse | human | purified | 100 | μg | R & D Systems |
| APOH | 350 | P02749 | apolipoprotein H (heta-2-glycoprotein 1) | 1D2 | mouse | human | tissue culture supernatant-liquid | 2 | mL | MyBiosource |
| APOH | 350 | P02749 | apolipoprotein H (beta-2-glycoprotein 1) | 9B20 | mouse | human | supernatant | 2 | ml | United States Biological |
| APOH | 350 | P02749 | apolipoprotein H (beta-2-glycoprotein 1) | 11C82 | mouse | human | purified by immunoaffinity | 100 | μg | United States Biological |
| APOL1 | 8542 | O14793 | apolipoprotein L-I | 1D4 | mouse | human | ascites | 0.1 | mL | Novus Biologicals |
| APOM | 55937 | PO95445 | apolipoprotein M | 3H3 | mouse | human | purified | 0.1 | mL | Orlgene Technologies |
| APOM | 55937 | PO95445 | apolipoprotein M | 10C3G5 | mouse | human | Protein A purified | 100 | μg | Novus Biologicals |
| APOM | 55937 | PO95445 | apolipoprotein M | 8F12C6B8 | mouse | human | Protein A purified | 100 | μg | Novus Biologicals |
| APOM | 55937 | PO95445 | apolipoprotein M | 10C3G5 | mouse | human | purified antibody in PBS | 100 | μg | ProMab Biotechnologies |
| APDM | 55937 | PO95445 | apolipoprotein M | 8F12C6B8 | mouse | human | purified antibody in PBS | 100 | μg | ProMab Biotechnologies |
| APOM | 55937 | PO95445 | apolipoprotein M | BB63 | mouse | human | purified by Protein A affinity | 100 | μg | United States Biological |
| APOM | 55937 | PO95445 | apolipoprotein M | 8B64 | mouse | human | purified by Protein A affinity | 100 | μg | United States Biological |
| APOM | 55937 | PO95445 | apolipoprotein M | BA107 | mouse | human | purified by Protein A affinity | 40 | μg | United States Biological |
| APOM | 55937 | PO95445 | apolipoprotein M | 1F10 | mouse | human | IgG purified | 100 | μg | Novus Biologicals |
| APOM | 55937 | PO95445 | apolipoprotein M | 2E5 | mouse | human | IgG purified | 100 | μg | Novus Biologicals |
| APOM | 55937 | PO95445 | apolipoprotein M | 1A2 | mouse | human | IgG purified | 100 | μg | Novus Biologicals |
| APOM | 55937 | PO95445 | apolipoproteln M | 1G9 | mouse | human | IgG purified | 100 | μg | Novus Biologicals |
| APOM | 55937 | PO95445 | apolipoprotein M | 2A8 | mouse | human | IgG purified | 100 | μg | Novus Biologicals |
| APOM | 55937 | PO95445 | apolipoprotein M | 3C7 | mouse | human | Igo purified | 100 | μg | Novus Biologicals |
| APOM | 55937 | PO95445 | apolipoprotein M | 4C7 | mouse | human | IgG purified | 100 | μg | Novus Biologicals |
| APOM | 55937 | PO95445 | apolipoprotein M | 6H3 | mouse | human | IgG purified | 100 | μg | Novus Biologicals |
| APOO | 79135 | Q9BUR5 | apolipoprotein O | 2F1 | mouse | human, mouse | ascitic fluid | 0.1 | ml | ProMab Biotechnologies |
| ATRN | 8455 | O75882 | Attractin | 9H8 | mouse | human | n.d. | 0.1 | ml | Thermo Scientific Pierce |
| BMP1 | 649 | P13497 | Bone morphogenetic protein 1 | | | | | | | |
| C1Q | 712 | P02745 | Complement C1 q subcomponent subunit ? | 3R9/2 | mouse | human | Protein A purified | 0.1 | mL | Novus Biologicals |
| C1QA | 712 | P02745 | Complement C1 q subcomponent subunit A | poly | mouse | human | purified | 50 | μg | United States Biological |
| C1Q | 713 | P02746 | Complement C1 q subcomponent subunit ? | 9G135 | mouse | human | purified by Protein A affinity | 50 | μg | United States Biological |
| C1QB | 713 | P02746 | Complement C1 q subcomponent subunit B | poly | mouse | human | purified | 50 | μg | United States Biological |
| C1Q | 714 | P02747 | Complement C1 q subcomponent subunit ? | 10B372 | mouse | human | purified by Protein A affinity | 0.1 | mL | United States Biological |
| C1Q | 714 | P02747 | Complement C1 q subcomponent subunit ? | 10B371 | mouse | human | purified | 100 | μg | United States Biological |
| C1Q | 714 | P02747 | Complement C1 q subcomponent subunit ? | 9j110 | mouse | human | purified | 100 | μg | United States Biological |
| C1QC | 714 | P02747 | Complement C1 q subcomponent subunit C | 5F91 | mouse | human | purified | 100 | μg | United States Biological |
| C1R | 715 | P00736 | Complement C1 r subcomponent | 269104 | mouse | human | purified | 100 | μg | R & D Systems |
| C1S | 716 | P09871 | Complement C1 s subcomponent | 306904 | mouse | human | purified | 100 | μg | R & D Systems |
| C1S | 716 | P09871 | Complement C1 s subcomponent | 9G173 | mouse | human | Protein A purified | 100 | μg | United States Biological |
| C1S | 716 | P09871 | Complement C1 s subcomponent | 9G174 | mouse | human | Protein A purified | 100 | μg | United States Biological |
| C1S | 716 | P09871 | Complement C1 s subcomponent | 6F44 | mouse | human | Protein A purified | 100 | μg | United States Biological |
| C1S | 716 | P09871 | Complement C1 s subcomponent | 6F45 | mouse | human | Protein A purified | 100 | μg | United States Biological |
| C2 | 717 | P06681 | Complement C2 | 269716 | mouse | human | purified | 100 | μg | R & D Systems |
| C2 | 717 | P06681 | Complement C2 | 6F61 | mouse | human | purified by Protein A affinity | 100 | μg | United States Biological |
| C2 | 717 | P06681 | Complement C2 | 6F60 | mouse | human | purified by Protein A affinity | 100 | μg | United States Biological |
| C2 | 717 | P06681 | Complement C2 | 6F62 | mouse | human | purified by Protein A affinity | 100 | μg | United States Biological |
| C3 | 718 | P01024 | Complement C3 | 6F64 | mouse | human | purified by Protein A affinity | 100 | μg | United States Biological |
| C3 | 718 | P01024 | Complement C3 | 8J442 | mouse | human | purified by Protein G affinity | 50 | μg | United States Biological |
| C3 | 718 | P01024 | Complement C3 | 8J444 | mouse | human | purified | 50 | μg | United States Biological |
| C3 | 718 | P01024 | Complement C3 | 8J443 | mouse | human | purified | 50 | μg | United States Biological |
| C3 | 718 | P01024 | Complement C3 | 7C10 | mouse | human | protein A/G purified, BSA-free | 1 | mg | Bioporto Diagnostics |
| C3 | 718 | P01024 | Complement C3 | 2D8 | mouse | human | protein A/G purified, BSA-free | 1 | mg | Bioporto Diagnostics |
| C3 | 718 | P01024 | Complement C3 | 6F6 | mouse | human | protein A/G purified, BSA-free | 1 | mg | Bioporto Diagnostics |
| C3 | 718 | P01024 | Complement C3 | 10A1 | mouse | human | affinity purified | 100 | μg | Novus Biologicals |
| C3 | 718 | P01024 | Complement C3 | 28A1 | mouse | human | affinity purified | 100 | μg | Novus Biologicals |
| C3 | 718 | P01024 | Complement C3 | 474 | mouse | human | IgG purified | 50 | μg | Novus Biologicals |
| C3 | 718 | P01024 | Complement C3 | 755 | mouse | human | IgG purified | 50 | μg | Novus Biologicals |
| C3 | 718 | P01024 | Complement C3 | 5F9 | mouse | human | IgG purified | 0.2 | mL | Novus Biologicals |
| C3 | 718 | P01024 | Complement C3 | 6F65 | mouse | human | purified by Protein A affinity | 100 | μg | United States Biological |
| C3 | 718 | P01024 | Complement C3 | 6F66 | mouse | human | purified by Protein A affinity | 100 | μg | United States Biological |
| C3 | 718 | P01024 | Complement C3 | 6F63 | mouse | human | purified by Protein A affinity | 100 | μg | United States Biological |
| C4A | 720 | P0C0L4 | Complement C4-A | 10B390 | mouse | human | purified by Protein G affinity | 200 | μg | United States Biological |
| C4A | 720 | P0C0L4 | Complement component C4 | 25.3F4 | mouse | human | protein A/G purified, BSA-free | 1 | mg | Bioporto Diagnostics |
| C4B | 721 | P0C0LS | Complement C4-B | 25300 | mouse | human | protein A/G purified, BSA-free | 1 | mg | Bioporto Diagnostics |
| C4B | 721 | P0C0LS | Complement Component 4d | 10B396 | mouse | human | purified by Protein A affinity | 100 | μg | United States Biological |
| C4B | 721 | P0C0L [symbol] | Complement Component 4d | 10B397 | mouse | human | purified by Protein A affinity | 100 | μg | United States Biological |
| C4B | 721 | P0C0LS | Complement Component 4c | 10B394 | mouse | human | purified by Protein A affinity | 100 | μg | United States Biological |
| | | | (Complement C4c, C4c) | | | | | | | |
| C4B | 721 | P0C0LS | Complement Component 4c | 10B395 | mouse | human | purified by Protein A affinity | 100 | μg | United States Biological |
| | | | (Complement C4c, C4c) | | | | | | | |
| C4B | 721 | P0C0LS | complement C4 | KT29 | mouse | human | affinity purified | 100 | μg | Novus Biologicals |
| C4B | 721 | P0C0LS | Complement C4 | 6F70 | mouse | human | purified by Protein A affinity | 100 | μg | United States Biological |
| C4B | 721 | P0C0LS | Complement C4 | 6F71 | mouse | human | purified by Protein A affinity | 100 | μg | United States Biological |
| C4BPA | 722 | P04003 | Complement CA binding protein alpha chain | 40823 | mouse | human | Protein A purified | 50 | μg | Novus Biologicals |
| C4BPA | 722 | P04003 | Complement C4 Binding Protein | 40824 | mouse | human | purified IgG-liquid | 100 | μg | MyBiosource |
| C4BPA | 722 | P04003 | Complement C4 Binding Protein | 10B393 | mouse | human | purified by Protein A affinity | 100 | μg | United States Biological |
| C4BPA | 722 | P04003 | Complement C4 Binding Protein | 1G9 | mouse | human | IgG purified | 100 | μg | Novus Biologicals |
| C5 | 727 | P01031 | Complement C5 | 295009 | mouse | human | biotin conjugate | 250 | μg | R & D Systems |
| C5 | 727 | P01031 | Complement C5 | 295003 | mouse | human | purified | 500 | μg | R & D Systems |
| C5 | 727 | P01031 | Complement C5 | 39725 | mouse | human | Protein A purified | 100 | μg | Novus Biologicals |
| C5 | 727 | P01031 | Complement C5 | 12F3 | mouse | human | protein A/G purified, BSA-free | 1 | mg | Bioporto Diagnostics |
| C5 | 727 | P01031 | Complement C5 | 11F6 | mouse | human | protein A/G purified, BSA-free | 1 | mg | Bioporto Diagnostics |
| C5 | 727 | P01031 | Complement C5 | 40821 | mouse | human | purified IgG-liquid | 100 | μg | MyBiosource |
| C5 | 727 | P01031 | Complement C5 | HCC 5.1 | mouse | human | μg (lyophilized) with 0.5% BSA + 0.0 [symbol] | 50 | μg | MyBiosource |
| C5 | 727 | P01031 | Complement C5 | 557 | mouse | human | Protein G purified | 50 | μg | Novus Biologicals |
| C5 | 727 | P01031 | Complement C5 | 81447 | mouse | human | purified by Protein G affinity | 50 | μg | United States Biological |
| C6 | 729 | P13671 | Complement C6 | 056B-214.2.4.2 | mouse | human | Protein A purified | 100 | μg | Novus Biologicals |
| C6 | 729 | P13671 | Complement C6 | 10B400 | mouse | human | purified by Protein A affinity | 100 | μg | United States Biological |
| C6 | 729 | P13571 | Complement C6 | 10B2760 | mouse | human | purified by Protein G affinity | 1 | ml | United States Biological |
| C7 | 730 | P10643 | Complement C7 | 10B2761 | mouse | human | purified by Protein G affinity | 1 | ml | United States Biological |
| C7 | 730 | P10643 | Complement C7 | 10B401 | mouse | human | purified by Protein A affinity | 100 | μg | United States Biological |
| C7 | 730 | P10643 | Complement C7 | 030-113.7.5.4 | mouse | human | Protein A purified | 100 | μg | Novus Biologicals |
| C8B | 732 | P07358 | Complement C8 | monoclonal | mouse | human | Protein A column purified | n.d. | | LifeSpan Biosciences |
| C8B | 732 | P07358 | Complement C8 | 10B402 | mouse | human | purified by Protein A affinity | 100 | μg | United States Biological |
| C8B | 732 | P07358 | Complement C8 | 056B-373 | mouse | human | purified IgG - liquid | 100 | μg | MyBiosource |
| C9 | 735 | P02748 | Complement C9 | X197 | mouse | human | n.d. | 100 | μg | Thermo Scientific Pierce |
| C9 | 735 | P02748 | Complement C9 | 22 | mouse | human | protein A/G purified, BSA-free | 1 | mg | Bioporto Diagnostics |
| C9 | 735 | P02748 | Complement C9 | 53 | mouse | human | protein A/G purified, BSA-free | 1 | mg | Bioporto Diagnostics |
| C9 | 735 | P02748 | Complement C9 | monoclonal | mouse | human | Protein A purified | 100 | μg | Novus Biologicals |
| CETP | 1071 | P11597 | Cholesteryl Ester Transfer Protein | ATM192 | mouse | human | Protein A purified | 01 | mL | Novus Biologicals |
| CETP | 1071 | P11597 | Cholesteryl Ester Transfer Protein | 5D2 | mouse | human | purified | 0.1 | mL | United States Biological |
| CETP | 1071 | P11597 | Cholesteryl Ester Transfer Protein | aa143-160 | mouse | human | ascites | 0.1 | mL | EMD Millipore |
| CFB | 629 | P00751 | Complement Factor B | 40826 | mouse | human | purified IgG - liquid | 100 | μg | MyBiosource |
| CFB | 629 | P00751 | Complement Factor B | 014III-33.2.4.3 | mouse | human | purified IgG - liquid | 100 | μg | MyBiosource |
| CFB | 629 | P00751 | Complement Factor B | 39730 | mouse | human | Protein A purified | 100 | μg | Novus Biologicals |
| CFB | 629 | P00751 | Complement Factor B | 10K167 | mouse | human | purified. | 100 | μg | United States Biological |
| CFB | 629 | P00751 | Complement Factor B | KT21 | mouse | human | affinity purified | 100 | μg | Novus Biologicals |
| CFB | 629 | P00751 | Complement Factor B | 10K159 | mouse | human | ascites | 100 | μg | United States Biological |
| CFB | 629 | P00751 | Complement Factor B | 10K168 | mouse | human | purified by Protein A affinity | 100 | μg | United States Biological |
| CFB | 629 | P00751 | Complement Factor B | 6E80 | mouse | human | purified by Protein A affinity | 100 | μg | United States Biological |
| CFB | 629 | P00751 | Complement Factor B | 6F79 | mouse | human | purified by Protein A affinity | 100 | μg | United States Biological |
| CFB | 629 | P00751 | Complement Factor B | 6F81 | mouse | human | purified by Protein A affinity | 100 | μg | United States Biological |
| CFB | 629 | P00751 | Complement Factor B | 032B-22.1.X | mouse | human | purified IgG - liquid | 100 | μg | MyBiosource |
| CFH | 3075 | P08603 | Complement Factor H | 6F84 | mouse | human | purified by Protein A affinity | 100 | μg | United States Biological |
| CFH | 3075 | P08603 | Complement Factor H | 6F85 | mouse | human | purified by Protein A affinity | 100 | μg | United States Biological |
| CFH | 3075 | P08603 | Complement Factor H | 6E86 | mouse | human | purified by Protein A affinity | 100 | μg | United States Biological |
| CFI | 3426 | P05156 | Complement Factor I | 271203 | mouse | human | purified | 100 | μg | R & D Systems |
| CFI | 3426 | P05156 | Complement Factor I | 6F87 | mouse | human | purified by Protein A affinity | 100 | μg | United States Biological |
| CFI | 3426 | P05156 | Complement Factor I | 6F88 | mouse | human | purified by Protein A affinity | 100 | μg | United States Biological |
| CFI | 3426 | P05156 | Complement Factor I | 1B3 | mouse | human | IgG purified | 100 | μg | Novus Biologicals |
| CFI | 3426 | P05156 | Complement Factor I | OX-21 | mouse | human | purified | 50 | μg | MyBiosource |
| CLEC3B | 7123 | P05452 | Tetranectin | 5B7 | mouse | human | n.d. | 200 | μg | Thermo Scientific Pierce |
| CLEC3B | 7123 | P05452 | Tetranectin | 10E3 | mouse | human | n.d. | 200 | μg | Thermo Scientific Pierce |
| CLEC3B | 7123 | P05452 | Tetranectin | 11F1 | mouse | human | lyophilized supernatant | 1 | mL | Accurate Chemical & |
| | | | | | | | | | | Scientific |
| CLEC3B | 7123 | P05452 | Tetra nectin | 5B7 | mouse | human | protein A/G purified, BSA-free | 1 | mg | Bioporto Diagnostics |
| CLEC3B | 7123 | P05452 | Tetranectin | 10E3 | mouse | human | protein A/G purified, BA4-free | 1 | mg | Bioporto Diagnostics |
| CLEC3B | 7123 | P05452 | Tetranectin | 6F9 | mouse | human | protein A/G purified, BSA-free | 1 | mg | Bioporto Diagnostics |
| CLEC3B | 7123 | P05452 | Tetranectin | 130-11 | mouse | human | purified | 200 | μg | Accurate Chemical & |
| | | | | | | | | | | Scientific |
| CLEC3B | 7123 | P05452 | Tetranectin | 130-13 | mouse | human | purified | 200 | μg | Accurate Chemical & |
| | | | | | | | | | | Scientific |
| CLEC3B | 7123 | P05452 | Tetranectin | 130-14 | mouse | human | purified | 200 | μg | Accurate Chemical & |
| | | | | | | | | | | Scientific |
| CLEC3B | 7123 | P05452 | Tetranectin | 6F193 | mouse | human | purified by Protein A affinity | 100 | μg | United States Biological |
| CLEC3B | 7123 | P05452 | Tetranectin | 6F216 | mouse | human | purified by Protein A affinity | 100 | μg | United States Biological |
| CLEC3B | 7123 | P05452 | Tetranectin | 6F192 | mouse | human | purified by Protein A affinity | 100 | μg | United States Biological |
| CLU | 1191 | P10909 | Clusterin | 1A11 | mouse | human | Supplied in PBS (pH 7.4) | 0.1 | ml | MyBiosource |
| CLU | 1191 | P10909 | Clusterin | 3R3/2 | mouse | human | n.d. | 100 | μg | Thermo Scientific Pierce |
| CLU | 1191 | P10909 | Clusterin | 350227 | mouse | human | purified | 100 | μg | R & D Systems |
| CLU | 1191 | P10909 | Clusterin | 350270 | mouse | human | purified | 500 | μg | R & D Systems |
| CLU | 1191 | P10909 | Clusterin | 350207 | mouse | human | biotin conjugate | 250 | μg | R & D Systems |
| CLU | 1191 | P10909 | Clusterin | 10B2265 | mouse | human | purified by Protein A affinity | 100 | μg | United States Biological |
| CLU | 1191 | P10909 | Clusterin | 1A11 | mouse | human | affinity purified, unconjugated | 0.1 | mL | Amrican Research Products |
| CLU | 1191 | P10909 | Clusterin | 5i231 | mouse | human | purified by affinity chromatography | 100 | μg | United States Biological |
| CLU | 1191 | P10909 | Clusterin | 7G24 | mouse | human | purified by Protein G affinity | 0.1 | mL | United States Biological |
| CLU | 1191 | P10909 | Clusterin | 6D316 | mouse | human | purified | 100 | μg | United States Biological |
| CLU | 1191 | P10909 | Clusterin | 7D1 | mouse | human | lyophilized supernatant | 0.1 | mL | Accurate Chemical & |
| | | | | | | | | | | Scientific |
| CLU | 1191 | P10909 | Clusterin | 2F12 | mouse | human | IgG purified | 100 | μg | Novus Biologicals |
| CLU | 1191 | P10909 | Clusterin | 10K81 | mouse | human | biotin conjugate | 250 | μg | United States Biological |
| CLU | 1191 | P10909 | Clusterin | 10K73 | mouse | human | purified by Protein G affinity | 500 | μg | United States Biological |
| CLU | 1191 | P10909 | Clusterin | Hs*3 | mouse | human | n.d. | 100 | μg | Thermo Scientific Pierce |
| CLU | 1191 | P10909 | Clusterin | CLI-9 | mouse | human | n.d. | 50 | μg | Thermo Scientific Pierce |
| CLU | 1191 | P10909 | Clusterin | 41D | mouse | human | purified | 100 | μg | EMD Millipore |
| CLU | 1191 | P10909 | Clusterin | B-S | mouse | human | purified | 200 | μg/ml | Santa Cruz Biotechnology |
| CLU | 1191 | P10909 | Clusterin | 0.T.18 | mouse | human | purified by Protein G affinity | 100 | μg | United States Biological |
| CP | 1356 | P00450 | Ceruloplasmin | 3B11 | mouse | human | n.d. | 0.1 | mL | Thermo Scientific Pierce |
| CPN2 | 1370 | P22792 | Caboxypeptidase N polypeptide 2 | | | | | | | |
| CRP | 1401 | P02741 | C-Reactive protein | 5A9 | mouse | human | Protein G purified | 0.1 | mL | Novus Biologicals |
| CRP | 1401 | P02741 | C-Reactive Protein | 2611018 | mouse | human | purified, protein A | n.d. | | AALTO BIO |
| | | | | | | | | | | REAGENTS LTD. |
| CRP | 1401 | P02741 | C-Reactive Protein | 2611028 | mouse | human | purified, protein A | n.d. | | AALTO BIO |
| | | | | | | | | | | REAGENTS LTD. |
| CRP | 1401 | P02741 | C-Reactive Protein | CRP103 | mouse | human | purified, unlabeled | 1 | mg | MyBiosource |
| CRP | 1401 | P02741 | C-Reactive Protein | C7 | mouse | human | Protein A purified | 200 | μg | Novus Biologicals |
| CRP | 1401 | P02741 | C-Reactive Protein | C3 | mouse | human | Protein A purified | 200 | μg | Novus Biologicals |
| CRP | 1401 | P02741 | C-Reactive Protein | C1 | mouse | human | Protein A purified | 200 | μg | Novus Biologicals |
| CRP | 1401 | P02741 | C-Reactive Protein | C2 | mouse | human | Protein A purified | 200 | μg | Novus Biologicals |
| CRP | 1401 | P02741 | C-Reactive Protein | C4 | mouse | human | Protein A purified | 200 | μg | Novus Biologicals |
| CRP | 1401 | P02741 | C-Reactive Protein | C5 | mouse | human | Protein A purified | 200 | μg | Novus Biologicals |
| CRP | 1401 | P02741 | C-Reactive Protein | C6 | mouse | human | Protein A purified | 500 | μg | Novus Biologicals |
| CRP | 1401 | P02741 | C-Reactive Protein | CRP169 | mouse | human | purified, unlabeled | 1 | mg | MyBiosource |
| CRP | 1401 | P02741 | C-Reactive Protein | CRP30 | mouse | human | purified, unlabeled | 1 | mg | MyBiosource |
| CRP | 1401 | P02741 | C-Reactive Protein | CRP36 | mouse | human | purified, unlabeled | 1 | mg | MyBiosource |
| CRP | 1401 | P02741 | C-Reactive Protein | CRP11 | mouse | human | purified, unlabeled | 1 | mg | MyBiosource |
| CRP | 1401 | P02741 | C-Reactive Protein | CRP135 | mouse | human | purified, unlabeled | 1 | mg | MyBiosource |
| CRP | 1401 | P02741 | C-Reactive Protein | 6402 | mouse | human | purified | >1 | mg | Medix Biochemica |
| CRP | 1401 | P02741 | C-Reactive Protein | 6403 | mouse | human | purified | >1 | mg | Medix Blochemica |
| CRP | 1401 | P02741 | C-Reactive Protein | 6404 | mouse | human | purified | >1 | mg | Medix Biochemica |
| CRP | 1401 | P02741 | C-Reactive Protein | 6405 | mouse | human | purified | >1 | mg | Medix Biochemica |
| CRP | 1401 | P02741 | C-Reactive Protein | 6407 | mouse | human | purified | >1 | mg | Medix Biochemica |
| CRP | 1401 | P02741 | C-Reactive Protein | 232007 | mouse | human | purified | 500 | μg | R & D Systems |
| CRP | 1401 | P02741 | C-Reactive Protein | B893M | mouse | human | purified, unlabeled | 1 | mg | MyBiosource |
| CRP | 1401 | P02741 | C-Reactive Protein | 232026 | mouse | human, | purified | 500 | μg | R & D Systems |
| | | | | | | mouse, porcine | | | | |
| CRP | 1401 | P02741 | C-Reactive Protein | 5A9 | mouse | human | Protein G column purified | n.d. | | LifeSpan Biosciences |
| CRP | 1401 | P02741 | C-Reactive Protein | 10K173 | mouse | human | purified by Protein A affinity | 100 | μg | United States Biological |
| CRP | 1401 | P02741 | C-Reactive Protein | 10K174 | mouse | human | purified by Protein G affinity | 100 | μg | United States Biological |
| CRP | 1401 | P02741 | C-Reactive Protein | 10K175 | mouse | human | purified by Protein G affinity | 100 | μg | United States Biological |
| CRP | 1401 | P02741 | C-Reactive Protein | SB78c | mouse | human | IgG purified | 1.0 | mg | Novus Biologicals |
| F13B | 2165 | P05160 | Coagulation factor XIII bera subunit | | | | | | | |
| F13B | 2165 | P05160 | Factor XIIIa | 3F177 | mouse | human | purified | 0.1 | mL | MyBiosource |
| F2 | 2147 | P00734 | Prothrombin | AHT-5020 | mouse | human | n.d. | 100 | μg | Cell Sciences |
| F2 | 2147 | P00734 | Prothrombin | AHP-5013 | mouse | human | n.d. | 100 | μg | Cell Sciences |
| F2 | 2147 | P00734 | Prothrombin | 5D10 | mouse | human | Protein A purified | 250 | μg | Novus Biologicals |
| F2 | 2147 | P00734 | Prothrombin | 5G9 | . mouse | human | Protein A purified | 125 | μg | Novus Biologicals |
| F2 | 2147 | P00734 | Prothrombin | BDID95 | mouse | human | purified | 500 | μg | MyBiosource |
| F8A | 8263 | P23610 | Coagulation factor VIII intron 22 protein | | | | | | | |
| F8A | 8263 | P23610 | Coagulation factor VIII intron 22 protein | 24-2-C7 | mouse | human | purified | 0.5 | mL | Accurate Chemical & |
| | | | | | | | | | | Scientific |
| F8A | 8263 | P23610 | Coagulation factor VIII intron 22 protein | SPM180 | mouse | human | n.d. | n.d. | | LifeSpan Biosciences |
| F8A | 8263 | P23610 | Coagulation factor VIII intron 22 protein | RFFVIRC/10 | mouse | human | purified IgG-liquid | 500 | μg | MyBiosource |
| F8A | 8263 | P23610 | Coagulation factor VIII intron 22 protein | RFFVIIIC/5 | mouse | human | purified IgG-liquid | 500 | μg | MyBiosource |
| F8A | 8263 | P23610 | Coagulation factor VIII intron 22 protein | 10C185 | mouse | human | purified | 500 | μg | United States Biological |
| F8A | 8263 | P23610 | Coagulation factor VIII intron 22 protein | 10C186 | mouse | human | purified | 500 | μg | United States Biological |
| F8A | 8263 | P23610 | Coagulation factor VIII intron 22 protein | 10C187 | mouse | human | supernatant | 5 | mL | United States Biological |
| F8A | 8263 | P23610 | Coagulation factor VIII intron 22 protein | AC-1A1 | mouse | human | IgG purified | 0.5 | mL | Novus Biologicals |
| F8A | 8263 | P23610 | Coagulation factor VIII intron 22 protein | RFF-VII/1 | mouse | human | affinity purified | 500 | μg | Novus Biologicals |
| F8A | 8253 | P23610 | Coagulation factor VIII intron 22 protein | RFF-VII/2 | mouse | human | affinity purified | 500 | μg | Novus Biologicals |
| F8A | 8263 | P23610 | Coagulation factor VIII intron 22 protein | AC-1A1 | mouse | human | n.d. | 500 | μg | Thermo Scientific Pierce |
| F8A | 8263 | P23610 | Coagulation factor VIII intron 22 protein | 4G5 | mouse | human | IgG purified | 0.2 | mL | Novus Biologicals |
| F8A | 8263 | P23610 | Coagulation factor VIII intron 22 protein | M1 | mouse | human | IgG purified | 100 | μg | Novus Biologicals |
| F8A | 8263 | P23610 | Coagulation factor VIII intron 22 protein | 8k144 | mouse | human | purified | 0.5 | mL | MyBiosource |
| F8A | 8263 | P23610 | Coagulation factor VIII intron 22 protein | 3F177 | mouse | human | purified | 0.1 | mL | MyBiosource |
| F8A | 8263 | P23610 | Coagulation factor VIII intron 22 protein | 9L234 | mouse | human | purified by Protein G affinity | 200 | μg | United States Biological |
| F8A | 8263 | P23610 | Coagulation factor VIII intron 22 protein | 9L236 | mouse | human | purified by Protein G affinity | 1 | mg | United States Biological |
| F8A | 8263 | P23610 | Coagulation factor VIII intron 22 protein | 9L237 | mouse | human | purified by Protein G affinity | 1 | mg | United States Biological |
| F8A | 8263 | P23610 | human Factor VIII, A2 Domain | 9L238 | mouse | human | Gel filtration and anion exchange chrom | 500 | μg | United States Biological |
| F8A | 8263 | P23610 | human Factor VIII, A2 Domain | 8k143 | mouse | human | purified | 100 | μg | United States Biological |
| FCN2 | 2220 | Q15485 | Ficolin-2 | 297018 | mouse | human | purified | 100 | μg | R & D Systems |
| FCN2 | 2220 | Q15485 | Ficolin-2 | 8H139 | mouse | human | purified by Protein G affinity | 100 | μg | United States Biological |
| FCN2 | 2220 | Q15485 | Ficolin-2 | 9B4 | mouse | human | purified by Protein A affinity | 100 | μg | United States Biological |
| FCN3 | 8547 | O75636 | Ficolin-3 | 296134 | mouse | human | purified | 100 | μg | R & D Systems |
| FCN3 | 8547 | O75636 | Ficolin-3 | FCN | mouse | human | Protein A purified | 100 | μg | Novus Biologicals |
| FCN3 | 8547 | O75636 | Ficolin-3 | 4B4 | mouse | human | IgG purified | 0.2 | mL | Novus Biologicals |
| FGA | 2243 | P02671 | Fibrinogen alpha chain | 1F7 | mouse | human | Protein A purified | 200 | μg | Novus Biologicals |
| FGA | 2243 | P02671 | Fibrinogen alpha chain | 26-E7 | mouse | human | Protein A purified | 200 | μg | Novus Biologicals |
| FGA | 2243 | P02671 | Fibrinogen alpha chain | 49D2 | mouse | human | Protein A purified | 200 | μg | Novus Biologicals |
| FGB | 2244 | P02675 | Fibrinogen beta chain | 3D1 | mouse | human | purified by Protein A affinity | 100 | μg | United States Biological |
| FGG | 2266 | P02679 | Fibrinogen gamma chain | 1F2 | mouse | human | IgG purified | 100 | μg | Novus Biologicals |
| FGG | 2266 | P02679 | Fibrinogen | 15H12 | mouse | human | Protein A purified | 100 | μg | Novus Biologicals |
| FGG | 2266 | P02979 | Fibrinogen | 1F9 | mouse | human | ascites | 0.1 | mL | Novus Biologicals |
| FGG | 2266 | P02679 | Fibrinogen | 1D6 | mouse | human | n.d. | 200 | μg | Thermo Scientific Pierce |
| FGG | 2266 | P02679 | Fibrinogen | 2F4 | mouse | human | n.d. | 200 | μg | Thermo Scientific Pierce |
| FGG | 2266 | P02679 | Fibrinogen | 5C5 | mouse | human | n.d. | 100 | μg | Thermo Scientific Pierce |
| FGG | 2266 | P02679 | Fibrinogen | 27CB | mouse | human | Protein A purified | 100 | μg | Novus Biologicals |
| FGG | 2266 | P02679 | Fibrinogen | 1SE11 | mouse | human | Protein A purified | 200 | μg | Novus Biologicals |
| FGG | 2266 | P02679 | Fibrinogen | 1F3 | mouse | human | Protein A purified | 200 | μg | Novus Biologicals |
| FGG | 2266 | P02679 | Fibrinogen | 41D9 | mouse | human | Protein A purified | 200 | μg | Novus Biologicals |
| FGG | 2266 | P02679 | Fibrinogen | 6G12 | mouse | human | Protein A purified | 200 | μg | Novus Biologicals |
| FGG | 2266 | P02679 | Fibrinogen | 1D12 | mouse | human | IgG purified | 100 | μg | Novus Biologicals |
| FN1 | 2335 | P02751 | Fibronectin 1 | SG7 | mouse | human | alkaline phosphatase (AP) conjugate | 1 | mL | Nouns Biologicals |
| FN1 | 2335 | P02751 | Fibronectin 1 | 2F12 | mouse | human | biotin conjugate | 100 | μg | Novus Biologicals |
| GC | 2638 | P02774 | Vitamin D-binding protein | 2B12 | mouse | human | n.d. | 0.1 | mL | Thermo Scientific Pierce |
| GC | 2638 | P02774 | Vitamin D-binding protein | 6F110 | mouse | human | purified by Protein A affinity | 100 | μg | United States Biological |
| GC | 2638 | P02774 | Vitamin D-binding protein | 6F109 | mouse | human | purified by Protein A affinity | 100 | μg | United States Biological |
| GC | 2638 | P02774 | Vitamin D-binding protein | 6F111 | mouse | human | purified by Protein A affinity | 100 | μg | United States Biological |
| GC | 2638 | P02774 | Vitamin D-binding protein | 6F108 | mouse | human | purified by Protein A affinity | 100 | μg | United States Biological |
| GC | 2638 | P02774 | Vitamin D-binding protein | 7H135 | mouse | human | purified by Protein G affinity | 100 | μg | United States Biological |
| GC | 2638 | P02774 | Vitamin D-binding protein | 249-01 | mouse | human | purified | 200 | μg | Accurate Chemical & |
| | | | | | | | | | | Scientific |
| GC | 2638 | P02774 | Vitamin D-binding protein | 249-01 | mouse | human | purified | 50 | μg. | Accurate Chemical & |
| | | | | | | | | | | Scientific |
| GC | 2638 | P02774 | Vitamin D-binding protein | 249-02 | mouse | human | purified | 200 | μg | Accurate Chemical & |
| | | | | | | | | | | Scientific |
| GC | 2638 | P02774 | Vitamin D-binding protein | 249-02 | mouse | human | purified | 50 | μg | Accurate Chemical & |
| | | | | | | | | | | Scientific |
| GC | 2638 | P02774 | Vitamin D-binding protein | 249-05 | mouse | human | purified | 200 | μg | Accurate Chemical & |
| | | | | | | | | | | Scientific |
| GC | 2638 | P02774 | Vitamin D-binding protein | 249-10 | mouse | human | purified | 200 | μg | Accurate Chemical & |
| | | | | | | | | | | Scientific |
| GC | 2638 | P02774 | Vitamin D-binding protein | 2G2 | mouse | human | n.d. | 200 | μg | Thermo Scientific Pierce |
| GC | 2638 | P02774 | Vitamin D-binding protein | 4B9 | mouse | human | n.d. | 200 | μg | Thermo Scientific Pierce |
| GSN | 2934 | P06396 | Gelsolin | 8L16 | mouse | human, | ascites | 0.1 | mL | MyBiosource |
| | | | | | | bovine, porcine | | | | |
| GSN | 2934 | P06396 | Gelsolin | GEL-42 | mouse | human, | affinity purified | 100 | μg | Insight Genomics |
| | | | | | | rabbit, bovine | | | | |
| GSN | 2934 | P06396 | Gelsolin | GS-2C4 | mouse | human, cow, | ascites | 0.1 | mL | Novus Biologicals |
| | | | | | | pig, rabbit | | | | |
| GSN | 2934 | P06396 | Gelsolin | 20 | mouse | human | n.d. | 0.2 | mL | Thermo Scientific Pierce |
| GSN | 2934 | P06396 | Gelsolin | 3SB2 | mouse | human | n.d. | 0.1 | mL | Thermo Scientific Pierce |
| GSN | 2934 | P06396 | Gelsolin | 3G5 | mouse | human | IgG purified | 100 | μg | Novus Biologicals |
| GSN | 2934 | P06396 | Gelsolin | 9j134 | mouse | human | purified by Protein A affinity | 100 | μg | United States Biological |
| GSN | 2934 | P06396 | Gelsolin | 10g36 | mouse | human, mouse, | ascites | 100 | μg | United States Biological |
| | | | | | | rabbit, [symbol] | | | | |
| GSN | 2934 | P06396 | Gelsolin | 20 | mouse | human | protein A/G purified, BSA-free | 1 | mg | Bioporto Diagnostics |
| GSN | 2934 | P06396 | Gelsolin | 10C345 | mouse | human | purified by Protein A affinity | 100 | μg | United States Biological |
| HP | 3240 | P00738 | Haptoglobin | 9G10 | mouse | human | n.d. | 200 | μg | Thermo Scientific Pierce |
| HP | 3240 | P00738 | Haptoglobin | 26E12 | mouse | human | n.d. | 0.1 | mL | Thermo Scientific Pierce |
| HP | 3240 | P00738 | Haptoglobin | 170-06 | mouse | human | purified | 200 | μg | Accurate Chemical & |
| | | | | | | | | | | Scientific |
| HP | 3240 | P00738 | Haptoglobin | HG-36 | mouse | human | n.d. | 0.5 | mL | Accurate Chemical & |
| | | | | | | | | | | Scientific |
| HP | 3240 | P00738 | Haptoglobin | 1.C.1 | mouse | human | ascites | 0.1 | mL | United States Biological |
| HP | 3240 | P00738 | Haptoglobin | 6F123 | mouse | human | purified by Protein A affinity | 100 | μg | United States Biological |
| HP | 3240 | P00738 | Haptoglobin | 9G10 | mouse | human | protein A/G purified, BSA-free | 1 | mg | Bioporto Diagnostics |
| HP | 3240 | P00738 | Haptoglobin | 2F4 | mouse | human | n.d. | 0.1 | mL | Thermo Scientific Pierce |
| HP | 3240 | P00738 | Haptoglobin | 1B1 | mouse | human | purified by affinity chromatography | 0.1 | mL | Origene Technologies |
| HP | 3240 | P00738 | Haptoglobin | 1F9 | mouse | human | purified by affinity chromatography | 0.1 | mL | Origene Technologies |
| HP | 3240 | P00738 | Haptoglobin | 2611 | mouse | human | purified by affinity chromatography | 0.1 | mL | Origene Technologies |
| HP | 3240 | P00738 | Haptoglobin | 268 | mouse | human | purified by affinity chromatography | 0.1 | mL | Origene Technologies |
| HP | 3240 | P00738 | Haptoglobin | 2B9 | mouse | human | purified by affinity chromatography | 0.1 | mL | Origene Technologies |
| HP | 3240 | P00738 | Haptoglobin | 4C2 | mouse | human | purified by affinity chromatography | 0.1 | mL | Origene Technologies |
| HP | 3240 | P00738 | Haptoglobin | 4H5 | mouse | human | purified by affinity chromatography | 0.1 | mL | Origene Technologies |
| HP | 3240 | P00738 | Haptoglobin | 6H2 | mouse | human | purified by affinity chromatography | 0.1 | mL | Origene Technologies |
| HPX | 3263 | P02790 | Hemopexin | 3A9-1A9 | mouse | human | IgG purified | 100 | μg | Novus Biologicals |
| HPX | 3263 | P02790 | Hemopexin | 4 | mouse | human | protein A/G purified-biotin conjugate | 100 | μg | Bioporto Diagnostics |
| HPX | 3263 | P02790 | Hemopexin | 32 | mouse | human | protein A/G purified-biotin conjugate | 100 | μg | Bioporto Diagnostics |
| HPX | 3263 | P02790 | Hemopexin | 6F124 | mouse | human | purified by Protein A affinity | 100 | μg | United States Biological |
| HPX | 3263 | P02790 | Hemopexin | 6F125 | mouse | human | purified by Protein A affinity | 100 | μg | United States Biological |
| HRG | 3273 | P04196 | Histidine-rich glycoprotein | monoclonal | mouse | human | unconjugated, purified | 200 | μg | Sino Biological |
| HRG | 3273 | P04196 | Histidine-rich glycoprotein | monoclonal | mouse | human | unconjugated, purified | 500 | μg | Sino Biological |
| HRG | 3273 | P04196 | Histidine-rich glycoprotein | monoclonal | mouse | human | unconjugated, purified | 200 | μg | Sino Biological |
| HRG | 3273 | P04196 | Histidine-rich glycoprotein | monoclonal | mouse | human | unconjugated, purified | 500 | μg | Sino Biological |
| HRG | 3273 | P04196 | Histidine-rich glycoprotein | 227901 | mouse | human | purified | 500 | μg | R & D Systems |
| IGFALS | 3483 | P35858 | Insulin-like growth factor binding protein acid | | | | | | | |
| | | | labile subunit | | | | | | | |
| ITIH1 | 3697 | P19827 | Inter-alpha-trypsin inhibitor heavy chain H1 | poly | mouse | human | Protein A purified | 50 | μg | Novus Biologicals |
| ITIH2 | 3698 | P19823 | inter-alpha-trypsin inhibitor heavy chain H2 | | | | | | | |
| ITIH3 | 3699 | Q06033 | Inter-alpha-trypsin inhibitor heavy chain H3 | | | | | | | |
| ITIH4 | 3700 | Q14624 | Inter-alpha-trypsin inhibitor heavy chain H4 | 45A12 | mouse | human | n.d, | 0.1 | mL | Thermo Scientific Pierce |
| ITIH4 | 3700 | Q14624 | inter-alpha Trypsin Inhibitor | 40B10 | mouse | human | n.d. | 0.1 | mL | Thermo Scientific Pierce |
| ITIH4 | 3700 | Q14624 | | | | | | | | |
| KLKB1 | 3818 | P03592 | Plasma kallikrein B1 | 1.B.715 | mouse | human | purified by Protein G affinity | 200 | μg | United States Biological |
| KLKB1 | 3818 | P03592 | Plasma kallikrein B1 | 13G11 | mouse | human | purified | 200 | μg | QED Bioscience |
| KLKB1 | 3818 | P03592 | Plasma kallikrein B1 | 3G2 | mouse | human | IgG purified | 100 | μg | Novus Biologicals |
| KLKB1 | 3818 | P03592 | Plasma kallikrein B1 | 3D1 | mouse | human | IgG purified | 100 | μg | Novus Biologicals |
| KNG1 | 3827 | P01042 | Kininogen-1 | 236012 | mouse | human | purified | 500 | μg | R & D Systems |
| KNG1 | 3827 | P01042 | Kininogen-1 | 207025 | mouse | human | purified | 500 | μg | R & D Systems |
| KNG1 | 3827 | P01042 | Kininogen-1 | 4A1 | mouse | human | IgG purified | 100 | μg | Novus Biologicals |
| KNG1 | 3827 | P01042 | Kininogen-1 | 24F9 | mouse | human | n.d. | 0.1 | mL | Thermo Scientific Pierce |
| KNG1 | 3827 | P01042 | Kininogen-1 | 236012 | mouse | human | purified | 500 | μg | R & D Systems |
| KNG1 | 3827 | P01042 | Kininogen-1 | 236005 | mouse | human | purified | 500 | μg | R & D Systems |
| KNG1 | 3827 | P01042 | Kininogen-1 | 2B5 | mouse | human | n.d. | 50 | μg | Thermo Scientific Pierce |
| KNG1 | 3827 | P01042 | Kininogen-1 | 2B5 | mouse | human | n.d. | 200 | μg | Thermo Scientific Pierce |
| KNG1 | 3827 | P01042 | Kininogen-1 | C11C1 | mouse | human | n.d. | 200 | μg | Thermo Scientific Pierce |
| KNG1 | 3827 | P01042 | Kininogen-1 | 2B5 | mouse | human | purified | 200 | μg | QED Bioscience |
| KNG1 | 3827 | P01042 | Kininogen-1 | 1.B.708 | mouse | human | purified by Protein G affinity | 200 | μg | United States Biological |
| KNG1 | 3827 | P01042 | Kininogen-1 | 1.B.709 | mouse | human | purified by Protein G affinity | 200 | μg | United States Biological |
| LCAT | 3931 | P04180 | Lecithin-cholesterol acyltransferase | | | | | | | |
| LGALS3BP | 3959 | Q08380 | Lectin galactoside-binding soluble 3 | 6D57 | mouse | human | purified | 100 | μg | MyBiosource |
| | | | binding protein | | | | | | | |
| LPA | 4018 | P08519 | Apolipoprotein(a) | 8F6A9,8H5C5 | mouse | human | ascitic fluid containing 0.03% sodium az | 0.1 | mL | ProMab Biotechnologies |
| LPA | 4018 | P08519 | Apolipoprotein(a) | 2Q2209 | mouse | human | ascites | 0.1 | mL | United States Biological |
| LRG1 | 116844 | P02750 | Leucine-rich alpha-2-glycoprotein | 2FS.A2 | mouse | human | affinity purified | 100 | μg | Novus Biologicals |
| LRG1 | 116844 | P02750 | Leucine-rich alpha-2-glycoprotein | 2000 | mouse | human | IgG purified | 100 | μg | Novus Biologicals |
| LRG1 | 116844 | P02750 | Leucine-rich alpha-2-glycoprotein | 1H1 | mouse | human | IgG purified | 100 | μg | Novus Biologicals |
| LUM | 4060 | P51884 | Lumican | 358022 | mouse | human, mouse | purified | 100 | μg | R & D Systems |
| LUM | 4060 | P51884 | Lumican | 669401 | mouse | human, mouse | purified | 100 | μg | R & D Systems |
| MASP1 | 5648 | P48740 | Mannan-binding lectin serine protease 1 | | | | | | | |
| | | | precursor | | | | | | | |
| ORM1 | 5004 | P02763 | Orosomucoid 1 (Alpha-1-acid glycoprotein 1) | 2F9-1F10 | mouse | human | IgG purified | 100 | μg | Novus Biologicals |
| ORM1 | 5004 | P02763 | Orosomucoid 1 (Alpha-l-acid glycoprotein 1) | 386131 | mouse | human | purified | 100 | μg | R & D Systems |
| ORM1 | 5004 | P02763 | Orosomucoid 1 (Alpha-1-acid glycoprotein 1) | 27A1 | mouse . | human | n.d. | 0.1 | mL | Thermo Scientific Pierce |
| ORM1 | 5004 | P02763 | Orosomucoid 1 (Alpha-l-acid glycoprotein 1) | AGP-47 | mouse | human | n.d. | 0.2 | mL | Accurate Chemical & |
| | | | | | | | | | | Scientific |
| ORM1 | 5004 | P02763 | Orosomucoid 1 (Alpha-1-acid glycoprotein 1) | 1.B.732 | mouse | human | purified | 1 | mg | MyBiosource |
| ORM1 | 5004 | P02763 | Orosomucoid 1 (Alpha-1-acid glycoprotein 1) | 9H10 | mouse | human | purified | 100 | μg | MyBiosource |
| ORM2 | 5005 | P19652 | Orosomucoid 2 (alpha-1-acid glycoprotein 2) | | | | | | | |
| PAFAH1B1 | 5048 | P43034 | Platelet-activating factor acetylhydrolase IB | G-3 | mouse | human | purified | 200 | μg/ml | Santa Cruz Biotechnology |
| | | | subunit alpha | | | | | | | |
| PAFAH1B1 | 5048 | P43034 | Platelet-activating factor acetylhydrolase IB | 5A5 | mouse | human | IgG purified | 100 | μg | Novus Biologicals |
| | | | subunit alpha | | | | | | | |
| PAFAH1B1 | 5048 | P43034 | Platelet-activating factor acetylhydrolase IB | 2C12 | mouse | human | IgG purified | 100 | μg | Novus Biologicals |
| | | | subunit alpha | | | | | | | |
| PAFAH1B1 | 5048 | P43034 | Platelet-activating factor acetylhydrolase IB | 9G377 | mouse | human | purified | 100 | μg | MyBiosource |
| | | | subunit alpha | | | | | | | |
| PCOLCE2 | 26577 | Q911KZ9 | Procollagen C-endopeptidase enhancer 2 | | | | | | | |
| PCYOX1 | 51449 | Q9UHG3 | Prenylcysteine oxidase 1 | 5E151 | mouse | human | purified | 50 | μg | United States Biological |
| PCYOX1 | 51449 | Q9UHG3 | Prenylcysteine oxidase 1 | aa1-506 | mouse | human | IgG purified | 50 | μg | LifeSpan Biosciences |
| PCYOX1 | 51449 | Q9UHG3 | Prenylcysteine oxidase 1 | 3D7-D8 | mouse | human | IgG purified | 100 | μg | Novus Biologicals |
| PGLYRP2 | 114770 | Q96PD5 | Peptidoglycan recognition protein 2 | 45G1 | mouse | human | affinity purified | 100 | μg | Novus Biologicals |
| PGLYRP2 | 114770 | Q96PD5 | Peptidoglycan recognition protein 2 | 10K303 | mouse | human | ascites | 100 | μg | United States Biological |
| PLA2G7 | 7941 | Q13093 | Platelet-activating factor acetylhydrolase | 589 | mouse | human | IgG purified | 100 | μg | Novus Biologicals |
| | | | (PAFA) | | | | | | | |
| PLA2G7 | 7941 | Q13093 | Platelet-activating factor acetylhydrolase | 5D1 | mouse | human | IgG purified | 100 | μg | Novus Biologicals |
| | | | (PAFA) | | | | | | | |
| PLG | 5340 | P08519 | Plasminogen | 4D2 | mouse | human | IgG purified | 200 | μg | Novus Biologicals |
| PLG | 5340 | P08519 | Plasminogen | SH3 | mouse | human | IgG purified | 200 | μg | Novus Biologicals |
| PLG | 5340 | P08519 | Plasminogen | 8F11 | mouse | human | IgG purified | 200 | μg | Novus Biologicals |
| PLG | 5340 | P08519 | Plasminogen | 9D8 | mouse | human | IgG purified | 200 | μg | Novus Biologicals |
| PLG | 5340 | P08519 | Plasminogen | 3C2 | mouse | human | IgG purified | 500 | μg | Novus Biologicals |
| PLG | 5340 | P08519 | Plasminogen | SBF1 | mouse | human | azide-free | n.d. | | Lifespan Biosciences |
| PLG | 5340 | P08519 | Plasminogen | 2F6-C6 | mouse | human | Protein A column purified | n.d. | | LifeSpan Biosciences |
| PLG | 5340 | P08519 | Plasminogen | 11B5-B2 | mouse | human | Protein G column purified | n.d. | | Lifespan Biosciences |
| PLG | 5340 | P08519 | Plasminogen | 13C3-B10 | mouse | human | Protein G column purified | n.d. | | LifeSpan Biosciences |
| PLG | 5340 | P08519 | Plasminogen | 1C10-F2 | mouse | human | Protein G column purified | n.d. | | LifeSpan Biosciences |
| PLG | 5340 | P08519 | Plasminogen | 4G6-B11 | mouse | human | Protein G column purified | n.d. | | LifeSpan Biosciences |
| PLG | 5340 | P08519 | Plasminogen | 9F9-C4 | mouse | human | Protein G column purified | n.d. | | LifeSpan Biosciences |
| PLG | 5340 | P08519 | Plasminogen | 270409 | mouse | human | purified | 100 | μg | R & D Systems |
| PLG | 5340 | P08519 | Plasminogen | 270412 | mouse | human | purified | 100 | μg | R & D Systems |
| PLG | 5340 | F08519 | Plasminogen | SBF1 C1.21 | mouse | human | IgG purified | 100 | μg | Novus Biologicals |
| PLTP | 5360 | P55058 | Phospolipid transfer protein | 2F3-G4 | mouse | human | IgG purified | 100 | μg | Novus Biologicals |
| PLTP | 5360 | P55058 | Phospolipid transfer protein | 11C579 | mouse | human | IgG purified | 100 | μg | United States Biological |
| PON1 | 5444 | P27169 | Serum paraoxonase/arylesterase 1 | 2H7 | mouse | human | IgG purified | 100 | μg | Novus Biologicals |
| PON1 | 5444 | P27169 | Serum paraoxonase/arylesterase 1 | 17A12 | mouse | human, | n.d. | 100 | μg | Thermo Scientific Pierce |
| PON3 | 5446 | Q15166 | Serum paraoxonase/lactonase 3 | | | mouse, rat | | | | |
| PPBP | 5473 | P02775 | Platelet basic protein | 3B9 | mouse | human | IgG purified | 100 | μg | Novus Biologicals |
| PPBP | 5473 | P02775 | Platelet basic protein | 59418 | mouse | human | purified | 500 | μg | R & D Systems |
| PPBP | 5473 | P02775 | Platelet basic protein | 5C7 | mouse | human | purified | 200 | μg | MyBiosource |
| PPBP | 5473 | P02775 | Platelet basic protein | 9L517 | mouse | human | purified by Protein A affinity | 200 | μg | United States Biological |
| PROS1 | 5627 | P07225 | Vitamin-K-dependent protein S | 391609 | mouse | human | purified | 100 | μg | R & D Systems |
| PROS1 | 5627 | P07225 | Vitamin-K-dependent protein S | 8D5 | mouse | human | n.d. | 200 | μg | Thermo Scientific Pierce |
| PROS1 | 5627 | P07225 | Vitamin-K-dependent protein S | 3D7 | mouse | human | IgG purified | 100 | μg | Novus Biologicals |
| RBP4 | 5950 | P02753 | Retinol-binding protein RBP4 | 1A2 | mouse | hu man | Protein G purified | 100 | μg | Novus Biologicals |
| RBP4 | 5950 | P02753 | Retinol-binding protein RBP4 | 5H9 | mouse | human | Protein G purified | 100 | μg | Novus Biologicals |
| RBP4 | 5950 | P02753 | Retinol-binding protein RBP4 | 1E3 | mouse | human | IgG purified | 100 | μg | Novus Biologicals |
| RBP4 | 5950 | P02753 | Retinol-binding protein RBP4 | 3D12 | mouse | human | IgG purified | 100 | μg | Novus Biologicals |
| RBP4 | 5950 | P02753 | Retinol-binding protein RBP4 | 1A8 | mouse | human | IgG purified | 100 | μg | Novus Biologicals |
| RBP4 | 5950 | P02753 | Retinol-binding protein RBP4 | 1E9 | mouse | human | IgG purified | 100 | μg | Novus Biologicals |
| RBP4 | 5950 | P02753 | Retinol-binding protein RBP4 | 3B1 | mouse | human | IgG purified | 100 | μg | Novus Biologicals |
| RBP4 | 5950 | P02753 | Retinol-binding protein RBP4 | 4D9 | mouse | human | IgG purified | 100 | μg | Novus Biologicals |
| RBP4 | 5950 | P02753 | Retinol-binding protein RBP4 | 4E7 | mouse | human | IgG purified | 100 | μg | Novus Biologicals |
| RBP4 | 5950 | P02753 | Retinol-binding protein RBP4 | 4H7 | mouse | human | IgG purified | 100 | μg | Novus Biologicals |
| RBP4 | 5950 | P02753 | Retinol-binding protein RBP4 | 4B10 | mouse | human | IgG purified | 100 | μg | Novus Biologicals |
| RBP4 | 5950 | P02753 | Retinol-binding protein RBP4 | RB42 | mouse | human | Protein A purified | 200 | μg | Novus Biologicals |
| RBP4 | 5950 | P02753 | Retinol-binding protein RBP4 | RB45 | mouse | human | Protein A purified | 200 | μg | Novus Biologicals |
| RBP4 | 5950 | P02753 | Retinol-binding protein RBP4 | RB48 | mouse | human | Protein A purified | 200 | μg | Novus Biologicals |
| RBP4 | 5950 | P02753 | Retinol-binding protein RBP4 | RB49 | mouse | human | Protein A purified | 200 | μg | Novus Biologicals |
| RBP4 | 5950 | P02753 | Retinol-binding protein RBP4 | RB51 | mouse | human | Protein A purified | 200 | μg | Novus Biologicals |
| RBP4 | 5950 | P02753 | Retinol-binding protein RBP4 | RB5S | mouse | human | Protein A purified | 200 | μg | Novus Biologicals |
| RBP4 | 5950 | P02753 | Retinol-binding protein RBP4 | AT2B4 | mouse | human | IgG purified | 0.1 | mL | Novus Biologicals |
| SAA1 | 6288 | P02735 | Serum amyloid A protein (SAA1 and SAA2) | 10B160 | mouse | human | purified by Protein A affinity | 200 | μg | United States Biological |
| SAA1 | 6288 | P02735 | Serum amyloid A protein (SAA1 and SAA2) | 3C11-2C1 | mouse | human | IgG purified | 100 | μg | Novus Biologicals |
| SAA1 | 6288 | P02735 | Serum amyloid A protein (SAA1 and SAA2) | SAA1 | mouse | human | Protein A purified | 200 | μg | Novus Biologicals |
| SAA1 | 6288 | P02735 | Serum amyloid A protein (SAA1 and SAA2) | SAA11 | mouse | human | Protein A purified | 200 | μg | Novus Biologicals |
| SAA1 | 6288 | P02735 | Serum amyloid A protein (SAA1 and SAA2) | SAA12 | mouse | human | Protein A purified | 200 | μg | Novus Biologicals |
| SAA1 | 6288 | P02735 | Serum amyloid A protein (SAA1 and SAA2) | SAA14 | mouse | human | Protein A purified | 200 | μg | Novus Biologicals |
| SAA1 | 6288 | P02735 | Serum amyloid A protein (SAA1 and SAA2) | SAA15 | mouse | human | Protein A purified | 200 | μg | Novus Biologicals |
| SAA1 | 6288 | P02735 | Serum amyloid A protein (SAA1 and SAA2) | SAA6 | mouse | human | Protein A purified | 200 | μg | Novus Biologicals |
| SAA1 | 6288 | P02735 | Serum amyloid A protein (SAA1 and SAA2) | SAA7 | mouse | human | Protein A purified | 200 | μg | Novus Biologicals |
| SAA1 | 6288 | P02735 | Serum amyloid A protein (SAA1 and SAA2) | mc1 | mouse | human | IgG purified | 1.0 | ml | Novus Biologicals |
| SAA1 | 6288 | P02735 | Serum amyloid A protein (SAA1 and SAA2) | Reu86.5 | mouse | human | Protein G purified | 0.5 | ml | Novus Biologicals |
| SAA1 | 6288 | P02735 | Serum amyloid A protein )SAA1 and SAA2) | mc1 | mouse | human | n.d. | 1 | mL | Thermo Scientific Pierce |
| SAA1 | 6288 | P02735 | Serum amyloid A protein (SAA1 and SAA2) | mc1 | mouse | human | n.d. | 1 | mL | MyBiosource |
| SAA1 | 6288 | P02735 | Serum amyloid A protein (SAA1 and SAA2) | 9L409 | mouse | human | purified by Protein A affinity | 1 | mg | United States Biological |
| SAA1 | 6288 | P02735 | Serum amyloid A protein (SAA1 and SAA2) | 9L410 | mouse | human | purified by Protein A affinity | 1 | mg | United States Biological |
| SAA1 | 6288 | P02735 | Serum amyloid A protein (SAA1 and SAA2) | B332A | mouse | human | purified, unlabeled | 1 | mg | MyBiosource |
| SAA1 | 6288 | P02735 | Serum amyloid A protein (SAA1 and SAA2) | B333A | mouse | human | purified, unlabeled | 1 | mg | MyBiosource |
| SAA1 | 6288 | P02735 | Serum amyloid A protein (SAA1 and SAA2) | B336A | mouse | human | purified, unlabeled | 1 | mg | MyBiosource |
| SAA4 | 6291 | P35542 | Serum amyloid A-4 protein | 3C11 | mouse | human | IgG purified | 100 | μg | Novus Biologicals |
| SERPINA1 | 5265 | P01009 | Serpin Peptidase Inhibitor Clade A Member 1 | 6F4 | mouse | human | purified by Protein A affinity | 100 | μg | United States Biological |
| | | | (alpha-1-antitrypsin) | | | | | | | |
| SERPINA1 | 5265 | P01009 | Serpin Peptidase Inhibitor Clade A Member 1 | 1.B.737 | mouse | human | purified by Protein G affinity | 200 | μg | United States Biological |
| | | | (alpha-1-antitrypsin) | | | | | | | |
| SERPINA1 | 5265 | P01009 | Serpin Peptidase Inhibitor Clade A Member 1 | 8.F.14 | mouse | human | purified | 500 | μg | United States Biological |
| | | | (alpha-1-antitrypsin) | | | | | | | |
| SERPINA1 | 5265 | P01009 | Serpin Peptidase Inhibitor Clade A Member 1 | 1.B.15 | mouse | human | purified by Protein A affinity | 1 | mg | United States Biological |
| | | | (alpha-1-antitrypsin) | | | | | | | |
| SERPINA1 | 5265 | P01009 | Serpin Peptidase Inhibitor Clade A Member 1 | 1.B.14 | mouse | human | purified by Protein G affinity | 500 | μg | United States Biological |
| | | | (alpha-1-antitrypsin) | | | | | | | |
| SERPINA1 | 5265 | P01009 | Serpin Peptidase Inhibitor Clade A Member 1 | 1.B.739 | mouse | human | purified by Protein G affinity | 1 | mg | United States Biological |
| | | | (alpha-1-arititrypsin) | | | | | | | |
| SERPINA1 | 5265 | P01009 | Serpin Peptidase Inhibitor Clade A Member 1 | IC2 | mouse | human | affinity purified | 0.1 | mL | Novus Biologicals |
| | | | (alpha-1-antitrypsin) | | | | | | | |
| SERPINA1 | 5265 | P01009 | Serpin Peptidase Inhibitor Clade A Member 1 | 3C5 | mouse | human | affinity purified | 0.1 | mL | Novus Biologicals |
| | | | (alpha-1-antitrypsin) | | | | | | | |
| SERPINA1 | 5265 | P01009 | Serpin Peptidase Inhibitor Clade A Member 1 | 11G2 | mouse | human, canine | affinity purified | 0.1 | mL | Novus Biologicals |
| | | | (alpha-1-antitrypsin) | | | | | | | |
| SERPINA1 | 5265 | P01009 | Serpin Peptidase Inhibitor Clade A Member 1 | 15H10 | mouse | human, | affinity purified | 0.1 | mL | Novus Biologicals |
| | | | (alpha-1-antitrypsin) | | | primate, canine | | | | |
| SERPINA1 | 5265 | P01009 | Serpin Peptidase Inhibitor Clade A Member 1 | 5B12 | mouse | human, | affinity purified | 0.1 | mL | Novus Biologicals |
| | | | (alpha-1-antitrypsin) | | | primate, canine | | | | |
| SERPINA1 | 5265 | P01009 | Serpin Peptidase Inhibitor Clade A Member 1 | 9A1 | mouse | human, | affinity purified | 0.1 | mL | Novus Biologicals |
| | | | (alpha-1-antitrypsin) | | | primate, canine | | | | |
| SERPINA1 | 5265 | P01009 | Serpin Peptidase Inhibitor Clade A Member 1 | 2B12 | mouse | human, mouse | ascites | 0.1 | mL | Novus Biologicals |
| | | | (alpha-1-antitrypsin) | | | | | | | |
| SERPINA1 | 5265 | P01009 | Serpin Peptidase Inhibitor Clade A Member 1 | 3B7 | mouse | human | Protein A purified | 125 | μg | Novus Biologicals |
| | | | (alpha-1-antitrypsin) | | | | | | | |
| SERPINA1 | 5265 | P01009 | Serpin Peptidase Inhibitor Clade A Member 1 | 6F5 | mouse | human | purified | 100 | μg | MyBiosource |
| | | | (alpha-1-antitrypsin) | | | | | | | |
| SERPINA1 | 5265 | P01009 | Serpin Peptidase Inhibitor Clade A Member 1 | 1AT | mouse | human | purified IgG - liquid | 1 | mg | MyBiosource |
| | | | (alpha-1-antitrypsin) | | | | | | | |
| SERPINA3 | 12 | P01011 | Serpin Peptidase Inhibitor Clade A Member 3 | ACT 14C7 | mouse | human | affinity purified | n.d. | | LifeSpan Biosciences |
| | | | (alpha-1-antichymotrypsin) | | | | | | | |
| SERPINA3 | 12 | P01011 | Serpin Peptidase Inhibitor Clade A Member 3 | 213907 | mouse | human | purified | 500 | μg | R & D Systems |
| | | | (alpha-1-antichymotrypsin) | | | | | | | |
| SERPINA3 | 12 | P01011 | Serpin Peptidase Inhibitor Clade A Member 3 | 3F [symbol] | mouse | human | Protein A purified | 100 | μg | Novus Biologicals |
| | | | (alpha-1-antichymotrypsin) | | | | | | | |
| SERPINA3 | 12 | P01011 | Serpin Peptidase Inhibitor Clade A Member 3 | 1E6 | mouse | human | IgG purified | 100 | μg | Novus Biologicals |
| | | | (alpha-1-antichymotnipsin) | | | | | | | |
| SERPINA3 | 12 | P01011 | Serpin Peptidase Inhibitor Clade A Member 3 | 1C10 | mouse | human | IgG purified | 100 | μg | Novus Biologicals |
| | | | (alpha-1-antichymotrypsin) | | | | | | | |
| SERPINA3 | 12 | P01011 | Serpin Peptidase Inhibitor Clade A Member 3 | 8J277 | mouse | human | purified by Protein G affinity | 200 | μg | United States Biological |
| | | | (alpha-1-antichymotrypsin) | | | | | | | |
| SERPINA3 | 12 | P01011 | Serpin Peptidase Inhibitor Clade A Member 3 | 10B144 | mouse | human | purified | 1 | mg | MyBiosource |
| | | | (alpha-1-antichymotrypsin) | | | | | | | |
| SERPINA4 | 5267 | P29622 | Serpin Peptidase Inhibitor Clade A Member 4 | 8H119 | mouse | human | purified by Protein G affinity | 500 | μg | United States Biological |
| | | | (kallistatin) | | | | | | | |
| SERPINA4 | 5267 | P29622 | Serpin Peptidase Inhibitor Clade A Member 4 | 209919 | mouse | human | purified | 500 | μg | R & D Systems |
| | | | (kallistatin) | | | | | | | |
| SERPINA4 | 5267 | P29622 | Serpin Peptidase Inhibitor Clade A Member 4 | 209930 | mouse | human | purified | 500 | μg | R & D Systems |
| | | | (kallistatin) | | | | | | | |
| SERPINA6 | 866 | P08185 | Serpin Peptidase Inhibitor Clade A Member 6 | 1D9 | mouse | human | IgG purified | 100 | μg | Novus Biologicals |
| | | | (corticosteroid binding glob [symbol] | | | | | | | |
| SERPINA6 | 866 | P08185 | Serpin Peptidase Inhibitor Clade A Member 6 | 1F11 | mouse | human | IgG purified | 100 | μg | Novus Biologicals |
| | | | (corticosteroid binding glob [symbol] | | | | | | | |
| SERPINA6 | 866 | P08185 | Serpin Peptidase Inhibitor Clade A Member 6 | 3B12 | mouse | human | IgG purified | 100 | μg | Novus Biologicals |
| | | | (corticosteroid binding glob [symbol] | | | | | | | |
| SERPINA6 | 866 | P08185 | Serpin Peptidase Inhibitor Clade A Member 6 | 3C12 | mouse | human | IgG purified | 100 | μg | Novus Biologicals |
| | | | (corticosteroid binding glob [symbol] | | | | | | | |
| SERPINC1 | 462 | P01008 | Serpin Peptidase Inhibitor Clade C Member 1 | 8D5 | mouse | human | purified by affinity chromatography | 0.1 | mL | Origene Technologies |
| | | | (antithrombin III) | | | | | | | |
| SERPINC1 | 462 | P01008 | Serpin Peptidase Inhibitor Clade C Member 1 | 4B3 | mouse | human | n.d. | 200 | μg | Thermo Scientific Pierce |
| | | | (antithrombin III) | | | | | | | |
| SERPINC1 | 462 | P01008 | Serpin Peptidase Inhibitor Clade C Member 1 | 7E11 | mouse | human | n.d. | 200 | μg | Thermo Scientific Pierce |
| | | | (antithrombin III) | | | | | | | |
| SERPINC1 | 462 | P01008 | Serpin Peptidase Inhibitor Clade C Member 1 | 8C12 | mouse | human | n.d. | 100 | μg | Thermo Scientific Pierce |
| | | | (antithrombin III) | | | | | | | |
| SERPINC1 | 462 | P01008 | Serpin Peptidase Inhibitor Clade C Member 1 | 2B12 | mouse | human | IgG purified | 0.2 | mL | Novus Biologicals |
| | | | (antithrombin III) | | | | | | | |
| SERPINC1 | 462 | P01008 | Serpin Peptidase Inhibitor Clade C Member 1 | 2B12 | mouse | human | IgG purified | 100 | μg | Novus Biologicals |
| | | | (antithrombin III) | | | | | | | |
| SERPINC1 | 462 | P01008 | Serpin Peptidase Inhibitor Clade C Member 1 | 8D5 | mouse | human | affinity purified | 0.1 | mL | Novus Biologicals |
| | | | (antithrombin III) | | | | | | | |
| SERPIND1 | 3053 | P05546 | Serpin Peptidase Inhibitor Clade D Member 1 | 373008 | mouse | human | purified | 100 | μg | R & D Systems |
| | | | (heparin cofactor II) | | | | | | | |
| SERPIND1 | 3053 | P05546 | Serpin Peptidase Inhibitor Clade D Member 1 | 74E5 | mouse | human | n.d. | 0.1 | mL | Thermo Scientific Pierce |
| | | | (heparin cofactor II) | | | | | | | |
| SERPINF1 | 5176 | P36955 | Serpin peptidase inhibitor Clade F Member 1 | 1C4 | mouse | human | Protein G purified | 0.1 | mL | Novus Biologicals |
| | | | (pigment epithelium-derived [symbol] | | | | | | | |
| SERPINF1 | 5176 | P36955 | Serpin peptidase inhibitor Clade F Member 1 | 1C4 | mouse | human | Protein G column purified | n.d. | | LifeSpan Biosciences |
| | | | (pigment epithelium-derived [symbol] | | | | | | | |
| SERPINF1 | 5176 | P36955 | Serpin peptidase inhibitor Clade F Member 1 | 187003 | mouse | human | purified | 500 | μg | R & D Systems |
| | | | (pigment epithelium-derived [symbol] | | | | | | | |
| SERPINF1 | 5176 | P36955 | Serpin peptidase inhibitor Clade F Member 1 | 1C4 | mouse | human | purified | 0.1 | mL | MyBiosource |
| | | | (pigment epithelium-derived [symbol] | | | | | | | |
| SERPINF1 | 5176 | P36955 | Serpin peptidase inhibitor Clade F Member 1 | 1C4 | mouse | human | purified. Supplied in PBS (pH 7.4) | 0.1 | mL | ATGen Ltd. |
| | | | (pigment epithelium-derived [symbol] | | | | | | | |
| SERPINF2 | 5345 | P08697 | Serpin peptidase inhibitor Clade F Member 2 | 236122 | mouse | human | purified | 500 | μg | R & D Systems |
| | | | (alpha-2-antiplasmin) | | | | | | | |
| SERPINF2 | 5345 | P08697 | Serpin peptidase inhibitor Clade F Member 2 | 9L37 | mouse | human | purified by Protein A affinity | 200 | μg | United States Biological |
| | | | (alpha-2-antiplasmin) | | | | | | | |
| SERPING1 | 710 | P05155 | Serpin peptidase inhibitor Clade G Member 1 | 7H118 | mouse | human | purified | 100 | μg | MyBiosource |
| | | | (plasma protease C1 inhibit [symbol] | | | | | | | |
| SERPING1 | 710 | P05155 | Serpin peptidase inhibitor Clade G Member 1 | 3F4-1D9 | mouse | human | purified. | 100 | μg | United States Biological |
| | | | (plasma protease C1 inhibit [symbol] | | | | | | | |
| SERPING1 | 710 | P05155 | Serpin peptidase inhibitor Clade G Member 1 | 10K343 | mouse | human | purified by Protein A affinity | 100 | μg | United States Biological |
| | | | (plasma protease C1 inhibit [symbol] | | | | | | | |
| SERPING1 | 710 | P05155 | Serpin peptidase inhibitor Clade G Member 1 | 10K344 | mouse | human | purified | 100 | μg | United States Biological |
| | | | (plasma protease C1 inhibit [symbol] | | | | | | | |
| SERPING1 | 710 | P05155 | Serpin peptidase inhibitor Clade G Member 1 | 7H118 | mouse | human | purified by Protein G affinity | 100 | μg | United States Biological |
| | | | (plasma protease C1 inhibit [symbol] | | | | | | | |
| SEPP1 | 6414 | P49908 | Selenoprotein P | 37A1 | mouse | human | n.d. | 100 | μg | Thermo Scientific Pierce |
| TF | 7018 | P02787 | Serotransferin | 5G2 | mouse | human | purified by affinity chromatography | 0.1 | mL | Origene Technologies |
| TF | 7018 | P02767 | Serotransferin | 29606 | mouse | human | purified | 500 | μg | R & D Systems |
| TF | 7018 | P02787 | Serotransferin | HTF-14 | mouse | human, rabbit | ion exchange chromatography purified | n.d. | | Lifespan Biosciences |
| TFPI | 7035 | P10646 | Tissue Factor Pathway Inhibitor | 374718 | mouse | human | purified | 500 | μg | R & D Systems |
| TFPI | 7035 | P10646 | Tissue Factor Pathway Inhibitor | 374720 | mouse | human | purified | 100 | μg | R & D Systems |
| TFPI | 7035 | P10645 | Tissue Factor Pathway Inhibitor | 3E8 | mouse | human | IgG purified | 100 | μg | Novus Biologicals |
| TTR | 7276 | P02766 | Transthyretin | 9G6 | mouse | human | affinity purified | 0.05 | mL | Novus Biologicals |
| TTR | 7276 | P02766 | Transthyretin | 7G8-E11-E7 | mouse | human | purified | 100 | μg | EMD Millipore |
| TTR | 7276 | P02766 | Transthyretin | 4D8 | mouse | human | IgG purified | 100 | μg | Novus Biologicals |
| TTR | 7276 | P02766 | Transthyretin | AT7B2 | mouse | human | liquid. In PBS (pH 7.4) with 0.1% sodium | 0.1 | mL | MyBiosource |
| VTN | 7448 | P04004 | Vitronectin | BV2 | mouse | human | purified IgG liquid | 100 | μg | MyBiosource |
| VTN | 7448 | P04004 | Vitronectin | 1E934 | mouse | human | purified, unlabeled | 1 | mg | MyBiosource |
| VTN | 7448 | P04004 | Vitronectin | 8 [symbol] 6 (1.110) | mouse | human | IgG purified | 0.025 | mL | Novus Biologicals |
| VTN | 7448 | P04004 | Vitronectin | BV1 | mouse | human | IgG purified | 0.2 | mL | Novus Biologicals |
| VTN | 7448 | P04004 | Vitronectin | BE10 | mouse | human | IgG purified | 50 | μg | Novus Biologicals |
| VTN | 7448 | P04004 | Vitronectin | NKI-M9 | mouse | human | affinity purified | 100 tests | | Novus Biologicals |
| VTN | 7448 | P04004 | Vitronectin | VN58-1 | mouse | human | n.d. | 100 | μg | Thermo Scientific Pierce |
| VTN | 7448 | P04004 | Vitronectin | VIT-2 | mouse | human | n.d. | 50 | μl | Thermo Scientific Pierce |
| VTN | 7448 | P04004 | Vitronectin | HV2 | mouse | human | n.d. | 200 | μg | Thermo Scientific Pierce |
| VTN | 7448 | P04004 | Vitronectin | HV23 | mouse | human | n.d. | 200 | μg | Thermo Scientific Pierce |
| VTN | 7448 | P04004 | Vitronectin | 10E12 | mouse | human | n.d. | 0.1 | mL | Thermo Scientific Pierce |
| VTN | 7448 | P04004 | Vitronectin | CSI003-D2 | mouse | human | purified | 200 | μg | Accurate Chemical & |
| | | | | | | | | | | Scientific |
| VTN | 7448 | P04004 | Vitronectin | CSI003-08 | mouse | human | purified | 200 | μg | Accurate Chemical & |
| | | | | | | | | | | Scientific |
| VTN | 7448 | P04004 | Vitronectin | CSI003-21 | mouse | human | purified | 200 | μg | Accurate Chemical & |
| | | | | | | | | | | Scientific |
| VTN | 7448 | P04004 | Vitronectin | CSI003-23 | mouse | human | purified | 200 | μg | Accurate Chemical & |
| | | | | | | | | | | Scientific |
| VTN | 7448 | P04004 | Vitronectin | BE6( [symbol] 8) | mouse | human | purified | 100 | μg | EMD Millipore |
| VTN | 7448 | P04004 | Vitronectin | BD33M | mouse | human | purified, unlabeled | 1 | mg | MyBiosource |
| VTN | 7448 | P04004 | Vitronectin | 342603 | mouse | human | purified | 100 | μg | R & D Systems |
| VTN | 7448 | P04004 | Vitronectin | 2Q557 | mouse | human | purified immunoglobulin | 100 | μg | United States Biological |
| VTN | 7448 | P04004 | Vitronectin | 7H101 | mouse | human | purified by Protein G affinity | 100 | μg | United States Biological |
| VTN | 7448 | P04004 | Vitronectin | 6F210 | mouse | human | purified by Protein A affinity | 100 | μg | United States Biological |
| VTN | 7448 | P04004 | Vitronectin | 9L794 | mouse | human | purified by Protein A affinity | 1 | mg | United States Biological |
| VTN | 7448 | P04004 | Vitronectin | 5G26 | mouse | human | purified by Protein G affinity | 100 | μg | United States Biological |
| VTN | 7448 | P04004 | Vitronectin | 2Q558 | mouse | human | purified by Protein G affinity | 100 | μg | United States Biological |
| VTN | 7448 | P04004 | Vitronectin | 9L765 | mouse | human | purified | 1 | mg | United States Biological |
| VTN | 7448 | P04004 | Vitronectin | 9L764 | mouse | human | purified by Protein A affinity | 200 | μg | United States Biological |
| VTN | 7448 | P04004 | Vitronectin | 9L795 | mouse | human | purified by Protein A affinity | 1 | mg | United States Biological |
| VTN | 7448 | P04004 | Vitronectin | 9L796 | mouse | human | purified by Protein A affinity | 1 | mg | United States Biological |
| VTN | 7448 | P04004 | Vitronectin | 1.B.725 | mouse | human | purified by Protein G affinity | 500 | μg | United States Biological |
| VTN | 7448 | P04004 | Vitronectin | 2VN | mouse | human | purified | n.d. | | Technoclone GmbH |
| VTN | 7448 | P04004 | Vitronectin | HV2 | mouse | human | protein A/G purified, BSA-free | 1 | mg | Bioporto Diagnostics |
| VTN | 7448 | P04004 | Vitronectin | 9F516 | mouse | human | ascites | 50 | [symbol] l | United States Biological |
| VTN | 7448 | P04004 | Vitronectin | 6A672 | mouse | human | purified by Protein G affinity | 100 | μg | United States Biological |
| VTN | 7448 | P04004 | Vitronectin | 5F87 | mouse | human | purified IgG. | 500 | μg | United States Biological |

[symbol]

indicates data missing or illegible when filed

What is claimed is:

1. A method for measuring the amount of a high density lipoprotein (HDL) subpopulation present in a sample, wherein each particle of the HDL subpopulation being measured is characterized by the presence of a plurality of defined protein epitopes, the method comprising performing a quantitative antibody-based assay on the sample, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of the HDL subpopulation in the sample.

2-34. (canceled)

35. A method for determining the likelihood of a subject's becoming afflicted with a disorder, wherein the disorder's likelihood of onset is characterized by an abnormal amount of a defined high density lipoprotein (HDL) subpopulation, and wherein each particle of the HDL subpopulation is characterized by the presence of a plurality of defined protein epitopes, the method comprising
(a) performing a quantitative antibody-based assay on an HDL-containing sample from the subject, wherein (i) the assay employs one or more capture/detection antibody pairs, (ii) the capture and detection antibodies in each pair are directed to different protein epitopes present on each particle of the HDL subpopulation, and (iii) each antibody pair is directed to a different set of epitopes than is each other antibody pair, thereby measuring the amount of the HDL subpopulation in the sample; and
(b) comparing the measured amount of HDL subpopulation in the subject's sample with a standard correlative with a known likelihood of the disorder's onset, thereby determining the likelihood of the subject's becoming afflicted with the disorder.

36-75. (canceled)

76. A kit for performing the method of claim 1 or 35, comprising (i) a solid substrate suitable for use in performing an antibody-based assay; (ii) a capture antibody operably affixed to the substrate; and (iii) in a separate compartment, a detection antibody, wherein the capture and detection antibodies are directed to different protein epitopes present on each particle of a predetermined HDL subpopulation.

77-80. (canceled)
